(19) United States
(12) Patent Application Publication
(10) Pub. No.: US 2007/0106359 A1
(43) Pub. Date: May 10, 2007

(54) Cardiac harness assembly for treating congestive heart failure and for pacing/sensing

(76) Inventors: Alan Schaer, San Jose, CA (US); Craig Mar, Fremont, CA (US); Anh Truong, San Jose, CA (US); Matthew G. Fishler, Sunnyvale, CA (US); Lilip Lau, Los Altos, CA (US)

Correspondence Address: FULWIDER PATTON LLP, HOWARD HUGHES CENTER, 6060 CENTER DRIVE, TENTH FLOOR, LOS ANGELES, CA, 90045, US

(21) Appl. No.: 11/588,190

(22) Filed: Oct. 26, 2006

Related U.S. Application Data
(63) Continuation-in-part of application No. 11/515,226, filed on Sep. 1, 2006.
Application No. 11/515,226 is a continuation-in-part of application No. 10/704,376, filed on Nov. 7, 2003, now Pat. No. 7,155,295.

(51) Int. Cl. A61N 1/04

(52) U.S. Cl. 607/129; 607/119

(57) ABSTRACT

A pace/sense electrode is associated with a cardiac harness for treating the heart. The pace/sense electrode is positioned on the epicardial surface of the heart, preferably under the cardiac harness, to provide pacing and sensing therapy to the heart. Compressive forces from the cardiac harness serve to hold the pace/sense electrode in place and to push the electrode into direct contact with the epicardial surface of the heart. Various means are provided for placing the pace/sense electrode under the cardiac harness in a minimally invasive procedure.

### CROSS-REFERENCES TO RELATED APPLICATIONS

This application is a continuation-in-part application of U.S. Ser. No. 11/515,226 filed Sep. 1, 2006, which is a continuation-in-part application of U.S. Ser. No. 10/704,376 filed Nov. 7, 2003, the entire contents of each are incorporated herein by reference. This application is related to U.S. Ser. Nos. 10/793,549; 10/777,451; 11/097,405; 10/931,449; 11/158,913; 10/795,574; 11/051,823; 10/858,995; 10/964,420; 11/002,609; 11/304,077; and 11/193,043, all of which are incorporated by reference.

### BACKGROUND OF THE INVENTION

The present invention relates to a device for treating heart failure. More specifically, the invention relates to a cardiac harness configured to be fit around at least a portion of a patient's heart. The cardiac harness includes electrodes attached to a power source for use in defibrillation or pacing/sensing.

Congestive heart failure (“CHF”) is characterized by the failure of the heart to pump blood at sufficient flow rates to meet the metabolic demand of tissues, especially the demand for oxygen. One characteristic of CHF is remodeling of at least portions of a patient's heart. Remodeling involves physical change to the size, shape and thickness of the heart wall. For example, a damaged left ventricle may have some localized thinning and stretching of a portion of the myocardium. The thinned portion of the myocardium often is functionally impaired, and other portions of the myocardium attempt to compensate. As a result, the other portions of the myocardium may expand so that the stroke volume of the ventricle is maintained notwithstanding the impaired zone of the myocardium. Such expansion may cause the left ventricle to assume a somewhat spherical shape.

Cardiac remodeling often subjects the heart wall to increased wall tension or stress, which further impairs the heart's functional performance. Often, the heart wall will dilate further in order to compensate for the impairment caused by such increased stress. Thus, a cycle can result, in which dilation leads to further dilation and greater functional impairment.

Historically, congestive heart failure has been managed with a variety of drugs. Devices have also been used to improve cardiac output. For example, left ventricular assist pumps help the heart to pump blood. Multi-chamber pacing has also been employed to optimally synchronize the beating of the heart chambers to improve cardiac output. Various skeletal muscles, such as the latissimus dorsi, have been used to assist ventricular pumping. Researchers and cardiac surgeons have also experimented with prosthetic “girdles” disposed around the heart. One such design is a prosthetic “sock” or “jacket” that is wrapped around the heart.

Patients suffering from congestive heart failure often are at risk to additional cardiac failures, including cardiac arrhythmias. When such arrhythmias occur, the heart must be shocked to return it to a normal cycle, typically by using a defibrillator. Implantable cardioverter/defibrillators (ICD's) are well known in the art and typically have a lead from the ICD connected to an electrode implanted in the right ventricle. Such electrodes are capable of delivering a defibrillating electrical shock from the ICD to the heart.

Other prior art devices have placed the electrodes on the epicardium at various locations, including on or near the epicardial surface of the right and left heart. These devices also are capable of distributing an electrical current from an implantable cardioverter/defibrillator for purposes of treating ventricular defibrillation or hemodynamically stable or unstable ventricular tachyarrhythmias.

Patients suffering from congestive heart failure may also suffer from cardiac failures, including bradycardia and tachycardia. Such disorders typically are treated by both pacemakers and implantable cardioverter/defibrillators. The pacemaker is a device that paces the heart with timed pacing pulses for use in the treatment of bradycardia, where the ventricular rate is too slow, or to treat cardiac rhythms that are too fast, i.e., anti-tachycardia pacing. As used herein, the term “pacemaker” is any cardiac rhythm management device with a pacing functionality, regardless of any other functions it may perform such as the delivery cardioversion or defibrillation shocks to terminate atrial or ventricular fibrillation. Particular forms and uses for pacing/sensing can be found in U.S. Pat. No. 6,574,506 (Kramer et al.) and U.S. Pat. No. 6,223,079 (Bakels et al.); and U.S. Publication No. 2003/0130702 (Kramer et al.) and U.S. Publication No. 2003/0195575 (Kramer et al.), the entire contents of which are incorporated herein by reference thereto.

The present invention solves the problems associated with prior art devices relating to a harness for treating congestive heart failure and placement of electrodes for use in defibrillation, or for use in pacing/sensing.

### SUMMARY OF THE INVENTION

The present invention includes a passive restraint device consisting of a wireform cardiac harness delivered through a mini-thoracotomy using a delivery system. In one embodiment, defibrillation electrodes/leads are attached directly onto the cardiac harness. There is a need to provide the cardiac harness in combination with epicardial pace/sense electrodes to provide optimal Cardiac Resynchronization Therapy (CRT) in patients with inter- and intra-ventricular contraction dyssynchrony. The pace/sense electrodes could be integrated into fixed positions on the harness, however, there is benefit to being able to adjust the position of the pace/sense electrodes relative to the harness once on the heart. While the harness configured with integrated pace/sense electrodes could be moved to some degree in an attempt to optimize the electrode position, it is assumed that the harness is deployed into an optimal position for passive restraint and that it would be undesirable to alter that position. The benefit of adjusting the pace/sense electrode position is largely related to where the electrodes are positioned once the harness is deployed. The pace/sense electrodes may be located over a tissue region where there is insufficient sensing or pacing ability (e.g., over fat, ischemic, fibrotic, or necrotic tissue), or where there is a sub-optimal resynchronization effect. Besides sensing and pacing for CRT applications, there may be benefit to altering the placement of one or more pace/sense electrodes relative to the harness for bradycardic pacing (e.g., for backup VVI pacing, or for chronic pacing in locations other than the RV apex, which is thought to exacerbate heart failure symptoms). There is a further benefit of moving one or more defibrillation electrodes (either in combination with or independent of one or more pace/sense electrodes) relative to the harness to alter the defibrillation vector, local voltage gradients, and/or impedance to improve the ability to defibrillate the heart. The embodiments disclosed herein relate to various means to provide pace/sense and/or defibrillation electrodes which are associated with the cardiac harness, yet are movable relative to the harness. Typically the terms “electrode” and “lead” are used to note a specific part of the device as a whole (“electrode” meaning the pace/sense electrode or the defibrillation electrode, and “lead” being the body of the device that contains everything else (conductors, insulation, connectors, etc.)). Sometimes, however, either term is used generically to refer to the lead/electrode device as a whole. This lead/electrode device may have a pace/sense electrode or defibrillation electrode or both.

One of the advantages of having a movable pace/sense electrode used in conjunction with a passive restraint device such as the disclosed cardiac harness, is that it allows not only pacing and defibrillation therapies, but also treats congestive heart failure two different ways at the same time. Congestive heart failure is treated by the cardiac harness by wall stress reduction and congestive heart failure also can be treated by biventricular pacing to increase heart pump efficiency. In fact, it is contemplated that these effects are not only additive, but may be synergistic in that the end results could be better than the individual contributions of the therapies separately. A further advantage of providing movable pace/sense leads independently of the cardiac harness allows for optimization of the pace/sense function, and in the case of the cardiac harness having defibrillation electrodes attached to it, one can decouple the pace/sense function from the defibrillation function, thus allowing one to optimally place both devices for optimal defibrillation therapy and pace/sense therapy. Integrated intravenous lead systems do not allow decoupling of the pace/sense function from the defibrillation function since they are integrated and the positions of the electrodes are fixed relative to each other. Thus, an important advantage of the present invention provides for the decoupling of the pace/sense function from the defibrillation function since the pace/sense electrodes can be moved independently to an optimal position on the heart during delivery.

In one embodiment, a single pace/sense electrode (with optional defibrillation electrode) is attached to a delivery member that allows it to be slipped under a previously delivered cardiac harness. In this embodiment, the tension of the harness provides the compression required for the pace/sense electrodes to firmly contact the heart tissue. It may be necessary to provide a surface area on the pace/sense electrode at least as wide as a cell on the cardiac harness to ensure a more even distribution of the compression. Preferably, a delivery member would be a flattened “paddle-like” member that offers a low profile and resists side-to-side movement during advancement. The delivery member may be similar to the current push arm used to deploy the cardiac harness, though it may benefit from being wider, and having less of a “nub” at the end, and being either stiffer or more flexible. Holes in the delivery member offer the ability to secure the pace/sense electrode to the member with a thread-like material (release lines) and release it once it is in the desired position under the cardiac harness. As with other embodiments to be described, it is beneficial to connect the proximal end of the pace/sense electrode to a pace/sense analyzer prior to releasing the pace/sense electrode from the delivery member. This allows the user to make positional adjustments as necessary to optimize the desired electrical performance and/or effect on resynchronization.

While the pace/sense electrode and delivery member could be manufactured and packaged together, it may be desirable to allow the user the ability to load a separate sterile pace/sense electrode into a sterile delivery member (in the sterile field) at the time of surgery. In one embodiment, the pace/sense electrode could be inserted under a loose release line mechanism on the delivery member that is then cinched down on the pace/sense electrode by the user prior to delivery.

In the embodiments just described, the pace/sense electrode is placed under the harness after the harness has been delivered. There is a benefit to having the separate pace/sense electrode be deployed onto the heart at the same time as the cardiac harness. The pace/sense electrodes could be laced to any of the same push arms as the cardiac harness, and released onto the heart at the same time as the cardiac harness. In another embodiment, the pace/sense electrodes could be laced directly to the cardiac harness (with or without the support of an independent set of push arms). In this case, the release lines attached to the pace/sense electrode and/or delivery member could be removed independently of the release lines that attach the push arms to the harness. This allows the user to adjust the harness and electrodes together after the harness is deployed and the primary harness delivery system removed. In another embodiment, the pace/sense electrodes could be laced to delivery members that are positioned under the cardiac harness, but are not attached to the harness. There is an added benefit of this configuration in that the delivery members provide support to the harness to help prevent row flipping and cell interlocks as the harness is advanced onto the heart. In another embodiment, the delivery members are attached to the same slider as the push arms laced to the cardiac harness and all release lines are connected to the same pull ring. In another embodiment, the delivery members are attached to a separate sliding mechanism, preferably in front of the slider to which the push arms are connected. Alternatively, there could be one sliding mechanism, but the delivery members could be detached from it after deployment onto the heart. At this point, usage of the delivery members would be similar to the case of having a separate sliding mechanism. Either way, the release lines from the pace/sense electrodes and the cardiac harness are connected to separate removal mechanisms. The pace/sense electrodes may be able to be released independently of the other electrodes. The delivery members may also be removed from the slider independently of one another. This allows the pace/sense electrodes to be advanced either ahead of or with the cardiac harness. It also allows the removal of the primary cardiac harness delivery system, leaving behind the delivery members attached to the pace/sense electrodes. Each pace/sense electrode may then be manipulated under the harness as necessary before being released from the delivery member in order to find the optimal position on the heart for the pace/sense therapy.

It should be noted that the same or similar pace/sense electrode delivery techniques described above could be used to deploy a pace/sense electrode onto any position on the surface of the heart, including the right or left atrium. There are particular advantages of being able to place a pace/sense electrode on the left atrial epicardial surface. As is typically recommended for CRT procedures, atrial sensing and optional pacing allows for improved timing between atrial and ventricular contractions (assuming a ventricular pace/sense electrode is present). Placement of a pace/sense electrode onto the atrial epicardial surface prevents the need for venous access to the right atrium, thus allowing the cardiothoracic surgeon to perform the whole procedure. It also allows the possibility of left atrial electrode placement, which is not feasible from a venous approach. Left atrial sensing and optional pacing particularly optimizes left atrial and left ventricular contraction timing.

In the described embodiments, consideration is made for the interaction of the cardiac harness and the pace/sense electrode, which relies on the tension of the harness to hold the electrode in place. It may be that once the harness and pace/sense electrode are fibrosed in place, little relative motion exists. However, this may not be the case thereby requiring features in the pace/sense electrode and/or the cardiac harness to minimize relative movement between the devices, or if relative motion exists, minimize the friction or propensity for material abrasion in the chronic setting. Because silicone rubber in its unaltered cured state can abrade against itself and against other materials, it may be important to utilize implantable materials in the cardiac harness, and/or the pace/sense electrodes that are positioned against it, that have more abrasion resistant surfaces. Examples of abrasion resistant materials include, but are not limited to, application of a lubricious silicone oil or hydrophilic coating to the lead body surface; silicone extruded tubing (e.g., platinum-cured Nusil 4755) which has the surface modified with plasma; oxidative reduction of the silicone surface to a silicon suboxide; plasma enhanced chemical vapor deposition of a silicon suboxide (these processes should reduce the tackiness of the surface and increase toughness); silicone extruded tubing that has a Teflon or Parylene deposited upon the surface; a sleeve of TEFLON or ePTFE over the surface of the pace/sense electrode (the material could also be used in place of silicone); matrix of braided or wound fibers (e.g., TEFLON, polypropylene, or polyester) or a matrix of an otherwise porous material (e.g., ePTFE), impregnated with silicone or another implantable elastic material; silicone extruded tubing with a layer of polyurethane (e.g., 55D polyurethane, a more lubricious and abrasion resist implantable material) over the surface (either as a sleeve slipped over the surface, a sleeve melted down onto the surface, or coextruded onto the surface); polyurethane used in place of silicone; and a chemical blend of silicone and polyurethane, such as Elast-Eon 2A, produced by Aortech Biomaterials plc. A pace/sense electrode covered by an ePTFE sheet may not only reduce contact force (and frictional force), but the wireform harness may sink down to be flush with the top surface of the ePTFE, and the contact force (and frictional force) could be reduced to zero, thus eliminating the frictional and wear abrasion concerns between two devices in contact on a beating heart.

Mechanical features on the pace/sense electrode may help minimize migration of the pace/sense electrode placed adjacent the cardiac harness, and/or minimize relative movement between the materials that could cause material abrasion. One embodiment of a mechanical feature includes protrusions on the pace/sense electrode that are designed to hook within the harness wireforms and stabilize the pace/sense electrode relative to the harness. The protrusions are rounded, but could have any specific shape that would lend itself to securing each to the wireforms. During delivery, it may be possible to shield or cover the protrusions until the final position is determined. This could be done by covering the protrusions with material releasing the material with a release line. A retractable sleeve over the protrusions also could be used. Another embodiment would be to have the protrusions facing the side opposite the harness during delivery, and then torquing the pace/sense electrode to flip the protrusion up against the harness when the final or near-final pace/sense electrode position is attained.

Another embodiment of the pace/sense electrode is that it has a geometry in the region of the electrodes that is wider than the rest of the lead, preferably at least as wide as a hinge on the harness wireform, to help distribute the contact force of the harness against the pace/sense electrode. A reduction in contact force should help reduce the propensity of the material to abrade. Also, the material on the harness wireform side of the lead is preferably an abrasion resistant material, similar to those described above, but in this case preferably constructed from an ePTFE sheet. Besides being flexible and lubricious implantable material, the ePTFE has the advantage of allowing silicone, molded around the lead components, to impregnate its matrix and form a secure bond. An alternative to the ePTFE sheet would be a “fabric” or “mesh” of fibers, such as polyester. In one aspect of the invention, there are various materials that can be chosen for use on both the pace/sense electrode and cardiac harness to resist abrasion between the two. In addition, composite designs may also resist abrasion. Coils, braids, and/or weaves of metal (e.g., stainless steel, nitinol, platinum, MP35N), or abrasion-resistant polymers (e.g., polyester, polyimide, TEFLON, KEVLAR), may allow protection of the conductor and conductor insulation. The above materials may be incorporated within a matrix of polymer (e.g., silicone) within the pace/sense electrode. The outer layer of polymer may even be allowed to abrade as a sacrificial layer before the more abrasion-resistant material stops or significantly impedes further material loss. The key to avoiding abrasion is to limit the contact force and relative motion between the materials. A layer of material may be applied to the pace/sense electrode and/or harness that is expected to abrade and allow the mating materials to “sink into” one another. Thus the contact area between the materials will be increased from an initial point contact between curved surfaces to a more widespread contact surface. The benefit is that the local contact force between the materials will drop, and frictional (abrasive) forces will be reduced. The relative motion between the materials may also be reduced, further reducing potential for abrasion. A further aspect includes use of soft materials on the pace/sense electrodes and cardiac harness. The soft materials “sink into” one another, decreasing contact force and relative movement that can cause abrasion. Similar to constructions mentioned previously, material examples include a low durometer polymer, porous polymer, or brush/carpet-like material. In another aspect, the pace/sense electrodes are fixed relative to the cardiac harness, thereby preventing relative motion and substantially eliminating friction. As mentioned previously, any feature that helps secure the harness and pace/sense electrode together and prevent relative motion will help avoid abrasion.

If a porous material (e.g., fiber mesh, ePTFE, or other open cell polymer matrix) is used on the pace/sense electrode, the final open pore size may be optimized to achieve certain features of the pace/sense electrode, depending on where and how the pace/sense electrode is used. It may be desirable to limit the pore size to minimize tissue in-growth and facilitate later removal of the pace/sense electrode, or a portion of the pace/sense electrode, if it ever became necessary. However, in the region adjacent the cardiac harness wireforms, there may be an advantage of encouraging tissue in-growth that could serve to stabilize the pace/sense electrode and/or harness and minimize relative movement between the two.

There also may be an advantage to having the outer layer of the pace/sense electrode in contact with the harness and/or the material on the harness itself, consist of a soft material that compresses or dimples when the harness wireforms are pressed against it. This may help reduce the contact pressure between the pace/sense electrode and the harness, as well as to help the materials lock into one another, especially when fibrosed in place. In another aspect of the invention, the material at the interface of the cardiac harness and the pace/sense electrode could be made of a soft material that helps the harness settle into the lead material. This could be a porous or foam-like material, or a matrix of thin protrusions on the surface, to create a brush-like or carpet-like surface, into which the harness settles. In another aspect, the material at the interface could be made of a tacky material, such as a gel or low-durometer silicone, that helps the materials to stick to one another. In another aspect, the material on the pace/sense electrode and/or the cardiac harness could be designed to ensure that the tissue grows in and around the pace/sense electrode and harness, linking them together. Examples of such materials include ePTFE, DACRON, and porous silicone. Pore size could be 10-100 microns, preferably 20-30 micron. The above mentioned “brush” or “carpet-like” features also could enhance tissue in-growth. The material also could be selectively coated or impregnated with a drug that promotes fibrin deposition for an enhanced acute effect. In another aspect, an adhesive is applied between the pace/sense electrode and cardiac harness. The adhesive could be externally applied to the surface of the harness and pace/sense electrode just before or after deployment onto the heart.

In one aspect of the invention, a malleable retractor (or similar blunt, flat tool) is used to lift an already deployed harness (by placing the tool under the cardiac harness and lifting it away from the heart or turning it on its edge) and the pace/sense electrode is inserted under the tool. The tool serves to provide a clear path for inserting the electrode without hang-ups on the harness. Once the pace/sense electrode is under the harness the tool may be removed.

While the focus is on pacing, sensing, and defibrillation electrodes, the concepts may also be applied to any other sort of sensor placed on the heart (e.g., magnetic, ultrasound, pH, impedance, etc.).

One advantage of a pace/sense electrode not attached to the cardiac harness, is that it allows the physician to scout a position for the pace/sense electrode. This could be done before deploying the harness, after deploying the harness but before deploying the implantable electrode, or after deployment of both the harness and the implantable pace/sense electrode with the intent to move the implantable electrode to provide a better target. A combination of the above techniques also could be done. For example, the scout electrode could be used first to target a position, and then used again after deployment of the implantable pace/sense electrode to help confirm or adjust the proper position of the pace/sense electrode. Scouting involves moving an electrode around the surface of the heart to find a target location to position the implantable pace/sense electrode. This location is determined by a combination of the desired anatomic location of the electrode, the quality of the electro-gram, and the ability to pace the site. Importantly, one could use the same pace/sense electrode as that intended for permanent implantation, however, if the electrode contains a steroid eluting plug or collar, it may be important to provide a resorbable coating over the electrode to prevent early loss of the steroid before it is in the final implant position. Such a coating could be mannitol or polyethylene glycol (PEG). In another embodiment, one could use a non-implantable electrode probe to find the desired position. By not being permanently implanted, this probe may more easily incorporate the following features: cheaper to make and use; potentially reusable; easier to use; could be made with a specific feature to improve tissue contact (pre-shape curve, use of a steerable handle, or other stiffening/maneuvering mechanism); multi-electrode capability with a multi-pin connector to allow the ability to easily switch between electrodes at the proximal end (this would also allow the ability to connect to a multi-electrode mapping system, e.g., Bard EP, Pruka, Biosense, etc. for quick assessment of the ideal location); anatomic positioning could be enhanced with the incorporation of sensors to identify the position of the electrodes relative to the heart and relative to adequately conductive tissue. Examples of such sensors include magnetic hall sensors (such as used in the J&J/Biosense catheters), or ultrasound sensors (such as used in the Boston Scientific/Cardiac Pathways catheters).

In one aspect of the method of delivery, with some of the embodiments disclosed herein, the order of the deployment of the cardiac harness and the pace/sense electrodes may vary: deploy the pace/sense electrode then the cardiac harness; deploy the harness and the pace/sense electrodes at the same time; or deploy the harness then deploy the pace/sense electrodes.

In the disclosed embodiments, it is preferred that the implantable pace/sense electrode be deployed under the pericardium from an opening at the apex. However, it is possible that the electrode could be deployed from outside the pericardium. To accomplish this, an incision is made in the pericardium, somewhere other than at the apex, and the distal end of the pace/sense electrode is advanced onto the epicardium through the incision. The potential advantage of this approach would be to allow the pericardium to act as a means to prevent direct contact (that could cause material wear) between the pace/sense electrode body and harness.

The emphasis for the delivery systems listed below are on the implantable pace/sense electrode, but could apply to a non-implantable scouting probe as well. In one aspect of the invention, the pace/sense electrode has a lumen for receiving a guidewire. The pace/sense electrode is advanced over the guidewire which is atraumatic and has precise steering. The guidewire could be advanced atraumatically beyond the AV groove. In another aspect, the pace/sense electrode is attached to a delivery member with a release line. There is an additional benefit of using a release line to hold features of the pace/sense electrode (such as soft “wings” or “flaps” that extend beyond the location of the electrodes) tightly against the main body of the lead (e.g., wrapped around the lead body) with the release line. Securing the features allows easier deployment by preventing the features from interfering with the harness. Then, with either the same release line that is attached to the delivery member, or a separate line, the features may be released (or “unfurled” as the case may be) and allowed to take the intended shape against the heart and/or harness. In another aspect, a stylet is placed in a lumen in the pace/sense electrode for push force and torquability. The removable stylet could be straight or shaped round or flat. A stylet provides the ability to advance the pace/sense electrode, move it laterally, or to flip the pace/sense electrode over. A stylet can be inserted and removed from inside the pace/sense electrode to provide sufficient columnar support during advancement of the pace/sense electrode under the cardiac harness. In another aspect, use of a tool to create a space under the harness that allows the pace/sense electrode to be advanced without catching on the cardiac harness. Such a tool could be a malleable retractor, or other customized flat, stiff, low-profile tool to create the desired space.

Secure contact between the pace/sense electrode and myocardium is important for optimal sensing and pacing. The following features allow the ability to fix the pace/sense electrodes securely to the epicardial surface of the heart: use of the pericardium to hold the pace/sense electrode against the epicardial surface; use of the cardiac harness to compress the pace/sense electrode and/or pace/sense electrode body against the heart; or provide an expandable member on the pericardial side of pace/sense electrode (pace/sense electrode placed in space between epicardium and pericardium). If the pace/sense electrode is positioned on the outside of the harness, the expandable member expands against pericardium and forces electrodes into the epicardium. If the pace/sense electrode is positioned under the harness, the expandable member expands against the harness and possibly also the pericardium to force the electrodes onto the epicardium. Examples of an expandable member include an inflatable bladder (using air or fluid), or an expandable cage (e.g., nitinol wireforms). The member could be self-expanding or expanded by the user. Other features used to fix the pace/sense electrode include: tissue adhesive (a lumen in the pace/sense electrode with a distal port at one or more locations on the pace/sense electrode, including positions near the electrode, could be used to transport a tissue adhesive, e.g., cyanoacrylate, that would fix lead to the epicardial and/or pericardial tissue); pre-filled bladder of adhesive could also be punctured to allow the adhesive to dispense; elastic band (elasticity achieved through strain of a metal wireform such as the nitinol in the harness or with an elastic rubber-like polymer wherein the band would be attached to the lead and then made to elongate around the heart or relative to points/devices fixed relative to the heart); friction pads (the friction of features on the pace/sense electrode help hold the pace/sense electrode and/or electrodes against the heart surface).

In keeping with the invention, a cardiac harness and assembly is configured to fit at least a portion of a patient's heart and is associated with one or more electrodes capable of providing defibrillation and electrodes used for pacing and/or sensing functions. In one embodiment, an adapter having a cavity is used to retain one or more pacing/sensing electrodes. The adapter is configured to retain the pacing/sensing electrodes so that electrodes are placed in direct contact with the epicardial surface of the heart, or proximate the epicardial surface of the heart. The adapter has a cavity for receiving one or more pacing/sensing electrodes and in one embodiment, the cavity is sized and shaped for receiving the pacing/sensing electrodes in an interference fit. In other words, the pacing/sensing electrodes are pressed into the cavity of the adapter in a snap-fit relationship so that there is an interference fit without any further fastening means. In another embodiment, a fastener is used to securely retain the pacing/sensing electrodes in the cavity. In another embodiment, after the pace/sense electrodes are pressed into the cavity, silicone rubber or other dielectric material is molded over the pace/sense electrodes in order to further secure the electrodes in the cavity.

In one embodiment, the adapter resembles a clam shell configuration that has an opened and closed configuration. In the open configuration, the pace/sense electrodes are pressed into a cavity and the electrodes are retained in the adapter when the two halves of the clam shell configuration are moved to the closed position and fastened together. In another embodiment, the adapter is formed in two parts with the cavity formed in a first portion and in a second portion. The pace/sense electrodes are pressed into the cavity of either the first portion or second portion and then the first portion is mated to the second portion so that the cavity surrounds the pace/sense electrodes.

In the clam shell and two portion embodiments of the adapter, it is preferred that the cavity have apertures for receiving electrodes on the pace/sense electrodes. The electrodes typically are in the form of a small metal protrusion or button, such that the button or protrusion extends through the aperture in the cavity so that the metallic surface of the protrusion or button can come into direct contact with the surface of the heart, or come into nearly direct contact with the surface of the heart.

In one embodiment, the adapter includes a cavity for receiving a pace/sense electrode. After the pace/sense electrode is pressed into the cavity, a dielectric material is molded over the pace/sense electrode to retain the pace/sense electrode in the adapter. Preferably, the adapter is formed from a silicone rubber material as is the molded layer retaining the pace/sense electrode in the cavity. The electrode portion of the pace/sense electrode is not covered by dielectric material so that it can contact the heart directly.

The present invention also includes a method of delivery and a method of use of the adapter and the associated pace/sense electrodes in conjunction with a cardiac harness. In one embodiment, after the pace/sense electrodes have been attached to the adapter, the adapter assembly is positioned under an already implanted cardiac harness. Preferably, the adapter assembly is delivered minimally invasively to a desired position under the cardiac harness. In one embodiment, the adapter assembly is releasably attached to the distal end of a push arm which has an atraumatic distal end so that the push arm, with the adapter assembly attached thereto, can be advanced under the implanted cardiac harness without catching on or moving the cardiac harness. After the push arm has been used to position the adapter assembly (with the pacing/sensing electrodes attached thereto), the adapter assembly is released from the push arm and the push arm is removed from the body. Optionally, a malleable retractor is used to lift portions of the harness to create free or open space under the harness as the push arm and adapter assembly are advanced onto the heart. Since the cardiac harness has a number of rows of undulating hinges that surround the heart which impart a slight compressive pressure on the heart, the adapter assembly is held in position on the heart by the same compressive pressure without any further fastening means. Alternatively, a suture or other fastener can be used to more securely fasten the adapter assembly to the epicardial surface of the heart. The adapter assembly is positioned under the cardiac harness so that the electrodes on the pacing/sensing electrodes are facing the epicardial surface of the heart and preferably in direct contact with the heart. It is preferred that the adapter be formed of a dielectric material that is compatible with the material of the cardiac harness. In one embodiment, the cardiac harness is formed of a nitinol alloy wire that is coated with a silicone rubber. In this embodiment, the adapter is formed of a silicone rubber as well in order to reduce the frictional engagement between the adapter and the cardiac harness. Further, portions of the pacing/sensing electrodes also can be coated with a dielectric material compatible with the silicone rubber coating on the cardiac harness. Preferably, the pacing/sensing electrodes are also coated with silicone rubber or a similar material in order to reduce frictional engagement and reduce the likelihood of the development of abrasions thereby exposing the bare metal of the cardiac harness or any metal associated with the pacing/sensing lead. In another embodiment, the cardiac harness and the adapter have coatings of dissimilar materials to reduce frictional engagement.

The adapter and the associated pacing/sensing electrodes can be used with any of the embodiments disclosed herein. For example, in one embodiment, defibrillating electrodes are attached to the cardiac harness for providing a defibrillating shock to the heart. In this embodiment, after the cardiac harness with electrodes is mounted on the heart, the adapter assembly is positioned on the heart under the cardiac harness for the purpose of providing pacing/sensing functions. In another embodiment, the cardiac harness, without defibrillating electrodes, is mounted on the heart and the adapter assembly with pacing/sensing electrodes is placed under the cardiac harness for providing pacing/sensing therapy.

One embodiment of the method of use for the adapter and the associated pace/sense electrodes in conjunction with a cardiac harness includes fitting an existing pace/sense electrode into the adapter. An example of an existing pace/sense electrode is a Capsure Epi Lead manufactured by Medtronic, Inc., Minneapolis, Minn.

Other types of existing electrodes, such as a coronary sinus lead, can be retained by an adapter for placement against the surface of the heart and underneath a cardiac harness. An example of an existing coronary sinus lead is the Quicksite manufactured by St. Jude Medical, Inc.

In another embodiment, a moveable or modular pace/sense electrode spine or structure includes a spine body having a “paddle-like” shape that retains one bipolar pair of button type electrodes exposed on a front surface of the spine body. This moveable electrode spine may also be used in conjunction with a cardiac harness. It has also been contemplated that the moveable electrode spine retains only one electrode, and in use, multiple moveable electrodes may be positioned under the cardiac harness.

Another example of a moveable or modular pace/sense electrode spine or structure may include a spine body with a low profile having a general shape of a circle or other geometry that retains one bipolar pair of button electrodes exposed on a front surface of the spine body. The pair of electrodes may be placed side-by-side horizontally, linearly in a column, or diagonally.

Yet another example of a moveable or modular pace/sense electrode spine or structure includes a low profile, circular shaped spine body having an Omni directional bipolar electrode pair. All examples of the moveable electrode spine may include grip pads positioned on the front surface of the spine body, and the grip pads can take any shape, such as rectangular, square, or circular.

A moveable or modular defibrillation electrode may also be used in conjunction with a cardiac harness that is placed on a beating heart. In this embodiment a defibrillation lead having a lead body retains a defibrillation electrode coil for providing a defibrillating shock through the heart. The moveable defibrillation electrode may be useful in adding another electrode for defibrillation where an additional current vector would be useful to lower the defibrillation threshold.

In accordance with the present invention, a cardiac harness is configured to fit at least a portion of a patient's heart and is associated with one or more electrodes capable of providing defibrillation or pacing functions. In one embodiment, rows or strands of undulations are interconnected and associated with coils or defibrillation and/or pacing/sensing electrodes. In another embodiment, the cardiac harness includes a number of panels separated by coils or electrodes, wherein the panels have rows or strands of undulations interconnected together so that the panels can flex and can expand and retract circumferentially. The panels of the cardiac harness are coated with a dielectric coating to electrically insulate the panels from an electrical shock delivered through the electrodes. Further, the electrodes are at least partially coated with a dielectric material to insulate the electrodes from the cardiac harness. In one embodiment, the strands or rows of undulations are formed from Nitinol and are coated with a dielectric material such as silicone rubber. In this embodiment, the electrodes are at least partially coated with the same dielectric material of silicone rubber. The electrode portion of the leads are not covered by the dielectric material so that as the electrical shock is delivered by the electrodes to the epicardial surface of the heart, the coated panels and the portion of the electrodes that are coated are insulated by the silicone rubber. In other words, the heart received an electrical shock only where the bare metal of the electrodes are in contact with or are adjacent to the epicardial surface of the heart. The dielectric coating also serves to attach the panels to the electrodes.

In another embodiment, the electrodes have a first surface and a second surface, the first surface being in contact with the outer surface of the heart, such as the epicardium, and the second surface faces away from the heart. Both the first surface and the second surface do not have a dielectric coating so that an electrical charge can be delivered to the outer surface of the heart for defibrillating or for pacing. In this embodiment, at least a portion of the electrodes are coated with a dielectric coating, such as silicone rubber, Parylene™ or polyurethane. The dielectric coating serves to insulate the bare metal portions of the electrode from the cardiac harness, and also to provide attachment means for attaching the electrodes to the panels of the cardiac harness.

The number of electrodes and the number of panels forming the cardiac harness is a matter of choice. For example, in one embodiment the cardiac harness can include two panels separated by two electrodes. The electrodes would be positioned 180° apart, or in some other orientation so that the electrodes could be positioned to provide a optimum electrical shock to the epicardial surface of the heart, preferably adjacent the right ventricle or the left ventricle. In another embodiment, the electrodes can be positioned 180° apart so that the electrical shock carries through the myocardium adjacent the right ventricle thereby providing an optimal electrical shock for defibrillation or periodic shocks for pacing. In another embodiment, three leads are associated with the cardiac harness so that there are three panels separated by the three electrodes.

In yet another embodiment, four panels on the cardiac harness are separated by four electrodes. In this embodiment, two electrodes are positioned adjacent the left ventricle on or near the epicardial surface of the heart while the other two electrodes are positioned adjacent the right ventricle on or near the epicardial surface of the heart. As an electrical shock is delivered, it passes through the myocardium between the two sets of electrodes to shock the entire ventricles.

In another embodiment, there are more than four panels and more than four electrodes forming the cardiac harness. Placement of the electrodes and the panels is a matter of choice. Further, one or more electrodes may be deactivated.

In another embodiment, the cardiac harness includes multiple electrodes separating multiple panels. The embodiment also includes one or more pacing/sensing electrodes (multi-site) for use in sensing heart functions, and delivering pacing stimuli for resynchronization, including biventricular pacing and left ventricle pacing or right ventricular pacing.

In each of the embodiments, an electrical shock for defibrillation, or an electrical pacing stimuli for synchronization or pacing is delivered by a pulse generator, which can include an implantable cardioverter/defibrillator (ICD), a cardiac resynchronization therapy defibrillator (CRT-D), and/or a pacemaker. Further, in each of the foregoing embodiments, the cardiac harness can be associated with multiple pacing/sensing electrodes to provide multi-site pacing to control cardiac function. By associating multi-site pacing with the cardiac harness, the system can be used to treat contractile dysfunction while concurrently treating bradycardia and tachycardia. This will improve pumping function by altering heart chamber contraction sequences while maintaining pumping rate and rhythm. In one embodiment, pacing/sensing electrodes are positioned under the cardiac harness and on the epicardial surface of the heart adjacent to the left and right ventricle for pacing both the left and right ventricles.

In another embodiment, the cardiac harness includes multiple electrodes separating multiple panels. In this embodiment, at least some of the electrodes are positioned on or near (proximate) the epicardial surface of the heart for providing an electrical shock for defibrillation, and other of the electrodes are positioned on the epicardial surface of the heart to provide pacing stimuli useful in synchronizing the left and right ventricles, cardiac resynchronization therapy, and biventricular pacing or left ventricular pacing or right ventricular pacing.

In another embodiment, the cardiac harness includes multiple electrodes separating multiple panels. At least some of the electrodes provide an electrical shock for defibrillation, and one of the electrodes, a single site electrode, is used for pacing and sensing a single ventricle. For example, the single site electrode is used for left ventricular pacing or right ventricular pacing. The single site electrode also can be positioned near the septum in order to provide bi-ventricular pacing.

In yet another embodiment, the cardiac harness includes one or more electrodes associated with the cardiac harness for providing a pacing/sensing function. In this embodiment, a single site electrode is positioned on the epicardial surface of the heart adjacent the left ventricle for left ventricular pacing. Alternatively, a single site electrode is positioned on the surface of the heart adjacent the right ventricle to provide right ventricular pacing. Alternatively, more than one pacing/sensing electrode is positioned on the epicardial surface of the heart to treat synchrony of both ventricles, including bi-ventricular pacing.

In another embodiment, the cardiac harness includes coils that separate multiple panels. The coils have a high degree of flexibility, yet are capable of providing column strength so that the cardiac harness can be delivered by minimally invasive access.

All embodiments of the cardiac harness, including those with electrodes, are configured for delivery and implantation on the heart using minimally invasive approaches involving cardiac access through, for example, subxiphoid, subcostal, or intercostal incisions, and through the skin by percutaneous delivery using a catheter.

### BRIEF DESCRIPTION OF THE DRAWINGS

FIG. 1 depicts a schematic view of a heart with a prior art cardiac harness placed thereon.

FIGS. 2A-2B depict a spring hinge of a prior art cardiac harness in a relaxed position and under tension.

FIG. 3 depicts a prior art cardiac harness that has been cut out of a flat sheet of material.

FIG. 4 depicts the prior art cardiac harness of FIG. 3 formed into a shape configured to fit about a heart.

FIG. 5A depicts a flattened view of one embodiment of the cardiac harness of the invention showing two panels connected to two electrodes.

FIG. 5B depicts a cross-sectional view of an electrode.

FIG. 5C depicts a cross-sectional view of an electrode.

FIG. 6A depicts a cross-sectional view of an undulating strand or ring.

FIG. 6B depicts a cross-sectional view of an undulating strand or ring.

FIG. 6C depicts a cross-sectional view of an undulating strand or ring.

FIG. 7A depicts an enlarged plan view of a cardiac harness showing three electrodes separating three panels, with the far side panel not shown for clarity.

FIG. 7B depicts an enlarged partial plan view of the cardiac harness of FIG. 7A showing an electrode partially covered with a dielectric material which also serves to attach the panels to the electrode.

FIG. 8A depicts a transverse cross-sectional view of the heart showing the position of electrodes for defibrillation and/or pacing/sensing functions.

FIG. 8B depicts a transverse cross-sectional view of the heart showing the position of electrodes for defibrillation and/or pacing/sensing functions.

FIG. 8C depicts a transverse cross-sectional view of the heart showing the position of electrodes for defibrillation and/or pacing/sensing functions.

FIG. 8D depicts a transverse cross-sectional view of the heart showing the position of electrodes for defibrillation and/or pacing/sensing functions.

FIG. 9 depicts a plan view of one embodiment of a cardiac harness having panels separated by and attached to flexible coils.

FIG. 10 depicts a flattened plan view of a cardiac harness similar to that of FIG. 9 but with fewer panels and coils.

FIG. 11 depicts a plan view of one embodiment of a cardiac harness having panels separated by and attached to flexible coils.

FIG. 12 depicts a plan view of a cardiac harness similar to that shown in FIG. 11 mounted on the epicardial surface of the heart.

FIG. 13 depicts a perspective view of a cardiac harness similar to that of FIG. 9 where the harness has been folded to reduce its profile for minimally invasive delivery.

FIG. 14 depicts the cardiac harness of FIG. 13 in a partially bent and folded condition to reduce its profile for minimally invasive delivery.

FIG. 15A depicts an enlarged plan view of a cardiac harness showing continuous undulating strands with electrodes overlaying the strands.

FIG. 15B depicts an enlarged partial plan view of the cardiac harness of FIG. 15A showing continuous undulating strands with an electrode overlying the strands.

FIG. 15C depicts a partial cross-sectional view taken along lines 15C-15C showing the electrode and undulating strands.

FIG. 15D depicts a partial cross-sectional view taken along lines 15D-15D showing the undulating strands in notches in the electrode.

FIG. 16 depicts a top view of a fixture for winding wire to construct the cardiac harness.

FIG. 17 depicts a plan view of a portion of a cardiac harness showing panels separated by electrodes.

FIGS. 18A, 18B and 18C depict various views of a mold used for injecting a dielectric material around the cardiac harness and the electrodes.

FIGS. 19A, 19B and 19C depict various views of molds used in injecting a dielectric material around the cardiac harness and the electrodes.

FIG. 20 depicts a top view of a portion of an electrode having a metallic coil winding.

FIG. 21 depicts a side view of the electrode portion shown in FIG. 20.

FIG. 22 depicts a cross-sectional view taken along lines 22-22 showing lumens extending through the electrode.

FIG. 23 depicts a cross-sectional view taken along lines 23-23 depicting another embodiment of lumens extending through the electrode.

FIG. 24 depicts a top view of a portion of an electrode having multiple coil windings.

FIG. 25A depicts a side view of a portion of a defibrillator electrode combined with a pacing/sensing electrode.

FIG. 25B depicts a top view of the electrode portion of FIG. 25A.

FIGS. 26A-26C depict various views of a defibrillator electrode combined with a pacing/sensing electrode.

FIG. 27 depicts a side view of an introducer for delivering the cardiac harness through minimally invasive procedures.

FIG. 28 depicts a perspective end view of a dilator with the cardiac harness releasably positioned therein.

FIG. 29 depicts an end view of the introducer with the cardiac harness releasably positioned therein.

FIG. 30 depicts a schematic cross-sectional view of a human thorax with the cardiac harness system being delivered by a delivery device inserted through an intercostal space and contacting the heart.

FIG. 31 depicts a plan view of the heart with a suction device releasably attached to the apex of the heart.

FIG. 32 depicts a plan view of the heart with the suction device attached to the apex and the introducer positioned to deliver the cardiac harness over the heart.

FIG. 33 depicts a plan view of the cardiac harness being deployed from the introducer onto the epicardial surface of the heart.

FIG. 34 depicts a plan view of the heart with the cardiac harness being deployed from the introducer onto the epicardial surface of the heart.

FIG. 35 depicts a plan view of the heart with the cardiac harness having electrodes attached thereto, surrounding a portion of the heart.

FIG. 36 depicts a schematic view of the cardiac harness assembly mounted on the human heart together with leads and an ICD for use in defibrillation or pacing.

FIG. 37 depicts an exploded a side view of a delivery system with the introducer tube, dilator tube, and ejection tube shown prior to assembly.

FIG. 38 depicts a cross-sectional view of the introducer tube taken along lines 38-38.

FIG. 39 depicts a cross-sectional view taken along lines 39-39 showing the cross-section of the dilator tube.

FIG. 40 depicts a cross-sectional view taken along lines 40-40 extending through the plate of the ejection tube and showing the various lumens in the plate.

FIG. 41 depicts a cross-sectional view taken along lines 41-41 of the proximal end of the ejection tube.

FIG. 42 depicts a longitudinal cross-sectional view and schematic of the ejection tube with the leads from the electrodes extending through the lumens in the plate and the tubing from the suction cup extending through a lumen in the plate.

FIG. 43A is a plan view depicting the adapter with a cavity for receiving pacing/sensing electrodes.

FIGS. 43B and 43C are cross-sectional views taken along the lines 43B-43B and 43C-43C respectively depicting the adapter of FIG. 43A.

FIG. 44 is a plan view of the adapter depicting the outer surface of the adapter that faces away from the epicardial surface of the heart.

FIG. 45 is a plan view of the adapter depicting the cavity for receiving the pacing/sensing electrodes.

FIG. 46 is a plan view of the adapter depicting the surface facing away from the epicardial surface of the heart.

FIG. 47A is a plan view of an adapter depicting a clam shell configuration having a cavity for receiving pacing/sensing electrodes.

FIG. 47B is a plan view of an adapter depicting first and second mating portions.

FIG. 48 is a plan view an adapter depicting a cavity and a pair of pacing/sensing electrodes for insertion into the cavity.

FIG. 49 is a plan view depicting an adapter assembly in which a pair of pacing/sensing electrodes have been inserted into the cavities of the adapter.

FIG. 50A is a plan view depicting an adapter releasably attached to a pusher rod.

FIG. 50B is a front plan view depicting a malleable retractor for use with the push arm and adapter of FIG. 50A.

FIG. 50C is a side plan view depicting the malleable retractor of FIG. 50B.

FIG. 51 is an enlarged partial plan view depicting the distal end of the adapter assembly and push arm of FIG. 50.

FIG. 52 is a partial plan view depicting a cardiac harness assembly mounted on a heart with a push arm delivering an adapter assembly under the cardiac harness.

FIG. 53 is an enlarged partial plan view depicting an adapter assembly mounted on the epicardial surface of a heart and under the cardiac harness assembly.

FIG. 54 is an enlarged partial plan view depicting a cardiac harness and an adapter assembly mounted under the cardiac harness assembly.

FIG. 55 is a cross-sectional view depicting a human thorax with the adapter assembly being delivered by insertion through an intercostal space.

FIG. 56 is a plan view depicting a cardiac harness assembly mounted on a human heart with the adapter assembly being mounted on the epicardial surface of the heart under the cardiac harness assembly.

FIG. 57 is a plan view depicting the cardiac harness assembly mounted on a human heart with the adapter assembly mounted under the cardiac harness and the leads connected to an ICD for use in pacing/sensing.

FIG. 58A is a partial plan view depicting a pace/sense electrode with a stylet.

FIG. 58B is a side view depicting the pace/sense electrode of FIG. 58A showing protrusions on one side of the pace/sense electrode.

FIG. 59 is a cross-sectional view taken along lines 59-59 depicting the stylet lumen.

FIG. 60A is a partial plan view depicting a pace/sense electrode mounted on a delivery member and attached with release lines.

FIG. 60B is a side view depicting the pace/sense electrode and delivery member of FIG. 60A.

FIG. 61A is an exploded plan view depicting a pace/sense electrode for insertion into loops on a delivery member.

FIG. 61B is a plan view depicting the pace/sense electrode and delivery member of FIG. 61A with the loops being tightened around the pace/sense electrode.

FIG. 62 is a partial plan view depicting a pace/sense electrode having a lumen for receiving a guidewire.

FIG. 63 is a plan view depicting another embodiment of an adapter with a cavity for receiving a pacing/sensing electrode.

FIG. 63A is a perspective view depicting a sinus lead adapter retaining a bipolar coronary sinus lead.

FIG. 64 is a partial plan view of a modular pacing/sensing electrode spine.

FIG. 65 is a partial plan view of another embodiment of a moveable or modular pacing/sensing electrode spine.

FIG. 66 is a partial plan view of another embodiment of a moveable or modular pacing/sensing electrode spine.

FIG. 67 is a partial plan view of yet another embodiment of a modular pacing/sensing electrode spine.

FIG. 68 is a partial plan view of a modular defibrillation electrode spine.

FIG. 69 depicts a flattened plan view of a cardiac harness having panels separated by a spine that retains a defibrillation coil and two bipolar pairs of electrodes.

FIG. 70 depicts a flattened plan view of a another embodiment of a cardiac harness having panels separated by a spine that retains a defibrillation coil and one bipolar pair of electrodes.

FIG. 71 depicts a cross-sectional view of an electrode tip within a rubber cup during a silicone rubber molding process.

### DETAILED DESCRIPTION OF THE PREFERRED EMBODIMENTS

This invention relates to a method and apparatus for treating heart failure. It is anticipated that remodeling of a diseased heart can be resisted or even reversed by alleviating the wall stresses in such a heart. The present invention discloses embodiments and methods for supporting the cardiac wall and for providing defibrillation and/or pacing functions using the same system. Additional embodiments and aspects are also discussed in Applicants' co-pending application entitled “Multi-Panel Cardiac Harness” U.S. Ser. No. 60/458,991 filed Mar. 28, 2003, the entirety of which is hereby expressly incorporated by reference.

Prior Art Devices

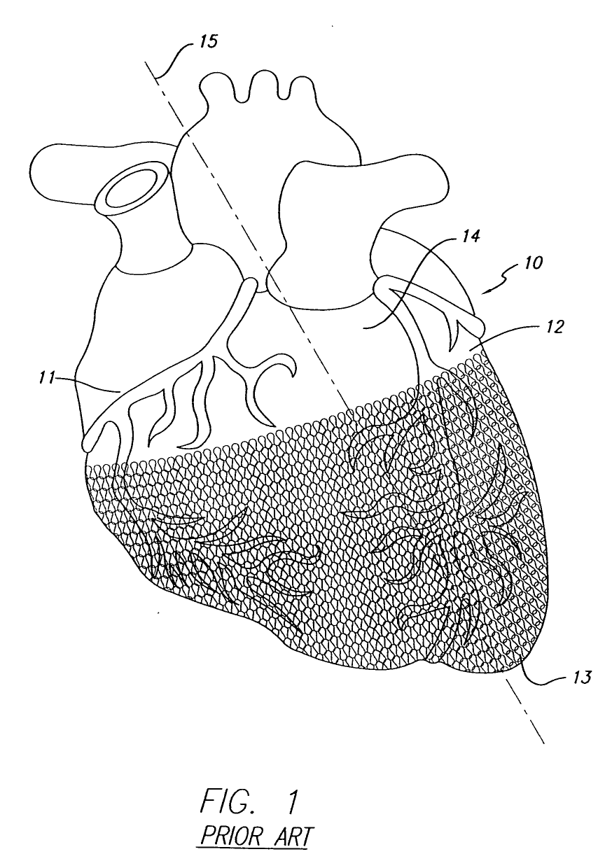

FIG. 1 illustrates a mammalian heart 10 having a prior art cardiac wall stress reduction device in the form of a harness applied to it. The harness surrounds a portion of the heart and covers the right ventricle 11, the left ventricle 12, and the apex 13. For convenience of reference, longitudinal axis 15 goes through the apex and the AV groove 14. The cardiac harness has a series of hinges or spring elements that circumscribe the heart and, collectively, apply a mild compressive force on the heart to alleviate wall stresses.

The term “cardiac harness” as used herein is a broad term that refers to a device fit onto a patient's heart to apply a compressive force on the heart during at least a portion of the cardiac cycle.

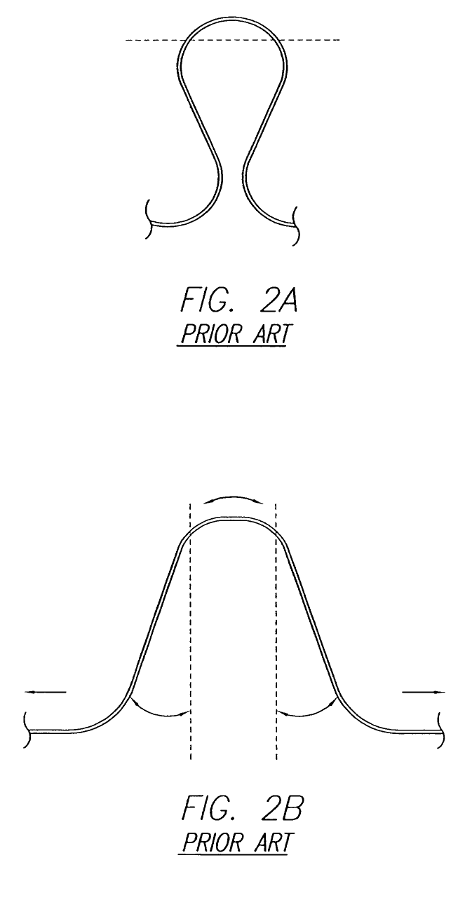

The cardiac harness illustrated in FIG. 1 has at least one undulating strand having a series of spring elements referred to as hinges or spring hinges that are configured to deform as the heart expands during filling. Each hinge provides substantially unidirectional elasticity, in that it acts in one direction and does not provide as much elasticity in the direction perpendicular to that direction. For example, FIG. 2A shows a prior art hinge member at rest. The hinge member has a central portion and a pair of arms. As the arms are pulled, as shown in FIG. 2B, a bending moment is imposed on the central portion. The bending moment urges the hinge member back to its relaxed condition. Note that a typical strand comprises a series of such hinges, and that the hinges are adapted to elastically expand and retract in the direction of the strand.

In the harness illustrated in FIG. 1, the strands of spring elements are constructed of extruded wire that is deformed to form the spring elements.

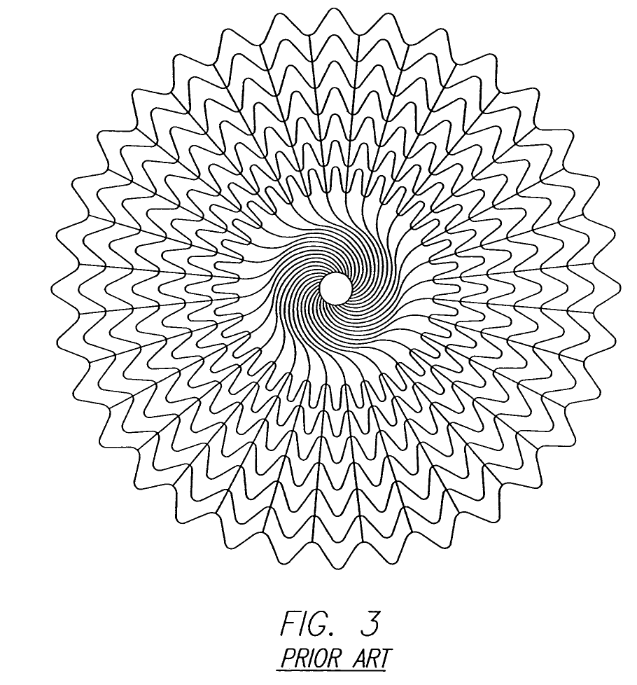

FIGS. 3 and 4 illustrate another prior art cardiac harness, shown at two points during manufacture of such a harness. The harness is first formed from a relatively thin, flat sheet of material. Any method can be used to form the harness from the flat sheet. For example, in one embodiment, the harness is photochemically etched from the material; in another embodiment, the harness is laser-cut from the thin sheet of material. The harness shown in FIGS. 3 and 4 has been etched from a thin sheet of Nitinol, which is superelastic material that also exhibits shape memory properties. The flat sheet of material is draped over a form, die or the like, and is formed to generally take on the shape of at least a portion of a heart.

With further reference to FIGS. 1 and 4, the cardiac harnesses have a base portion which is sized and configured to generally engage and fit onto a base region of a patient's heart, an apex portion which is sized and shaped so as to generally engage and fit on an apex region of a patient's heart, and a medial portion between the base and apex portions.

In the harness shown in FIGS. 3 and 4, the harness has strands or rows of undulating wire. As discussed above, the undulations have hinge/spring elements which are elastically bendable in a desired direction. Some of the strands are connected to each other by interconnecting elements. The interconnecting elements help maintain the position of the strands relative to one another. Preferably the interconnecting elements allow some relative movement between adjacent strands.

The undulating spring elements exert a force in resistance to expansion of the heart. Collectively, the force exerted by the spring elements tends toward compressing the heart, thus alleviating wall stresses in the heart as the heart expands. Accordingly, the harness helps to decrease the workload of the heart, enabling the heart to more effectively pump blood through the patient's body and enabling the heart an opportunity to heal itself. It should be understood that several arrangements and configurations of spring members can be used to create a mildly compressive force on the heart to reduce wall stresses. For example, spring members can be disposed over only a portion of the circumference of the heart or the spring members can cover a substantial portion of the heart.

As the heart expands and contracts during diastole and systole, the contractile cells of the myocardium expand and contract. In a diseased heart, the myocardium may expand such that the cells are distressed and lose at least some contractility. Distressed cells are less able to deal with the stresses of expansion and contraction. As such, the effectiveness of heart pumping decreases. Each series of spring hinges of the above cardiac harness embodiments is configured so that as the heart expands during diastole the spring hinges correspondingly will expand, thus storing expansion forces as bending energy in the spring. As such, the stress load on the myocardium is partially relieved by the harness. This reduction in stress helps the myocardium cells to remain healthy and/or regain health. As the heart contracts during systole, the disclosed prior art cardiac harnesses apply a moderate compressive force as the hinge or spring elements release the bending energy developed during expansion allowing the cardiac harness to follow the heart as it contracts and to apply contractile force as well.

Other structural configurations for cardiac harnesses exist, however, but all have drawbacks and do not function optimally to treat CHF and other related diseases or failures. The present invention cardiac harness provides a novel approach to treat CHF and provides electrodes associated with the harness to deliver an electrical shock for defibrillation or a pacing stimulus for resynchronization, or for biventricular pacing/sensing.

### The Present Invention Embodiments

The present invention is directed to a cardiac harness system for treating the heart. The cardiac harness system of the present invention couples a cardiac harness for treating the heart coupled with a cardiac rhythm management device. More particularly, the cardiac harness includes rows or undulating strands of spring elements that provide a compressive force on the heart during diastole and systole in order to relieve wall stress pressure on the heart. Associated with the cardiac harness is a cardiac rhythm management device for treating any number of irregularities in heart beat due to, among other reasons, congestive heart failure. Thus, the cardiac rhythm management device associated with the cardiac harness can include one or more of the following: an implantable cardioverter/defibrillator with associated leads and electrodes; a cardiac pacemaker including leads and electrodes used for sensing cardiac function and providing pacing stimuli to treat synchrony of both vessels; and a combined implantable cardioverter/defibrillator and pacemaker, with associated leads and electrodes to provide a defibrillation shock and/or pacing/sensing functions.

The cardiac harness system includes various configurations of panels connected together to at least partially surround the heart and assist the heart during diastole and systole. The cardiac harness system also includes one or more leads having electrodes associated with the cardiac harness and a source of electrical energy supplied to the electrodes for delivering a defibrillating shock or pacing stimuli.

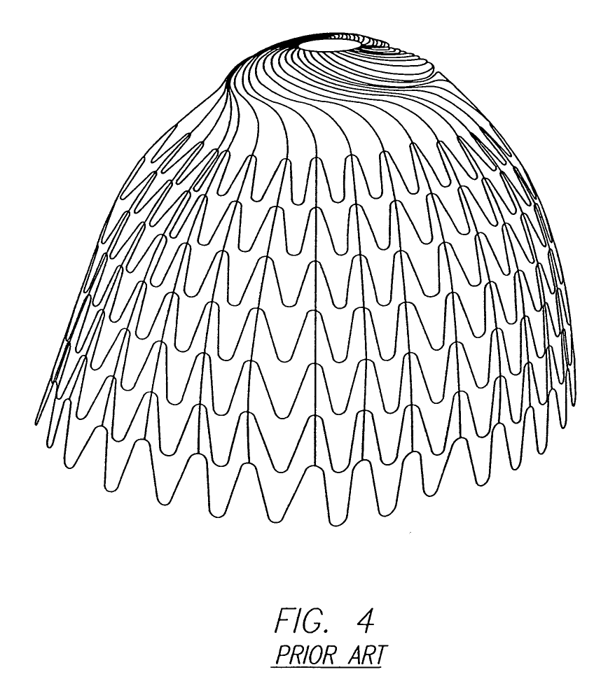

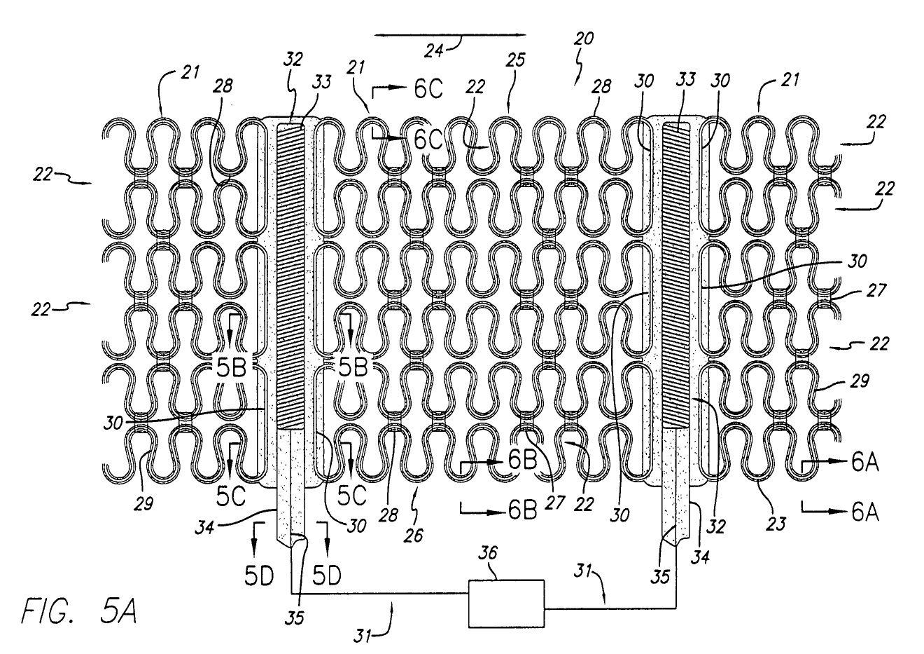

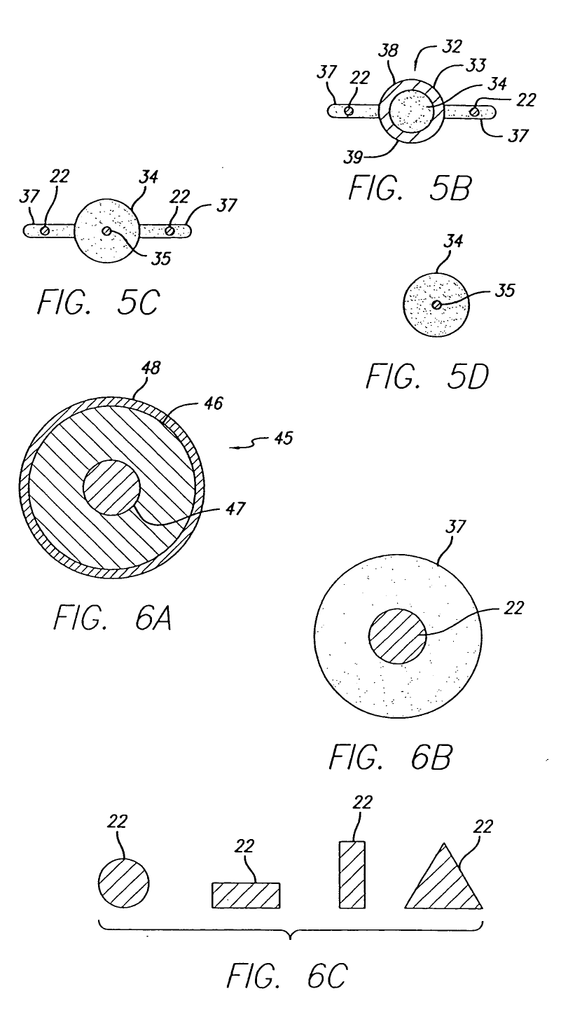

In one embodiment of the invention, as shown in a flattened configuration in FIG. 5, a cardiac harness 20 includes two panels 21 of generally continuous undulating strands 22. A panel includes rows or undulating strands of hinges or spring elements that are connected together and that are positioned between a pair of electrodes, the rows or undulations being highly elastic in the circumferential direction and, to a lesser extent, in the longitudinal direction. In this embodiment, the undulating strands have U-shaped hinges or spring elements 23 capable of expanding and contracting circumferentially along directional line 24. The cardiac harness has a base or upper end 25 and an apex or lower end 26. The undulating strands are highly elastic in the circumferential direction when placed around the heart 10, and to a lesser degree in a direction parallel to the longitudinal axis 15 of the heart. Similar hinges or spring elements are disclosed in co-pending and co-assigned U.S. Ser. No. 60/458,991 filed Mar. 28, 2003, the entire contents of which are incorporated herein by reference. While the FIG. 5 embodiment appears flat for ease of reference, in use it is substantially cylindrical (or tapered) to conform to the heart and the right and left side panels would actually be one panel and there would be no discontinuity in the undulating strands.

The undulating strands 22 provide a compressive force on the epicardial surface of the heart thereby relieving wall stress. In particular, the spring elements 23 expand and contract circumferentially as the heart expands and contracts during the diastolic and systolic functions. As the heart expands, the spring elements expand and resist expansion as they continue to open and store expansion forces. During systole, as the heart 10 contracts, the spring elements will contract circumferentially by releasing the stored bending forces thereby assisting in both the diastolic and systolic function.

As just discussed, bending stresses are absorbed by the spring elements 23 during diastole and are stored in the elements as bending energy. During systole, when the heart pumps, the heart muscles contract and the heart becomes smaller. Simultaneously, bending energy stored within the spring elements 23 is at least partially released, thereby providing an assist to the heart during systole. In a preferred embodiment, the compressive force exerted on the heart by the spring elements of the harness comprises about 10% to 15% of the mechanical work done as the heart contracts during systole. Although the harness is not intended to replace ventricular pumping, the harness does substantially assist the heart during systole.

The undulating strands 22 can have varying numbers of spring element 23 depending upon the amplitude and pitch of the spring elements. For example, by varying the amplitude of the pitch of the spring elements, the number of undulations per panel will vary as well. It may be desired to increase the amount of compressive force the cardiac harness 20 imparts on the epicardial surface of the heart, therefore the present invention provides for panels that have spring elements with lower amplitudes and a shorter pitch, thereby increasing the expansion force imparted by the spring element. In other words, all other factors being constant, a spring element having a relatively lower amplitude will be more rigid and resist opening, thereby storing more bending forces during diastole. Further, if the pitch is smaller, there will be more spring elements per unit of length along the undulating strand, thereby increasing the overall bending force stored during diastole, and released during systole. Other factors that will affect the compressive force imparted by the cardiac harness onto the epicardial surface of the heart include the shape of the spring elements, the diameter and shape of the wire forming the undulating strands, and the material comprising the strands.

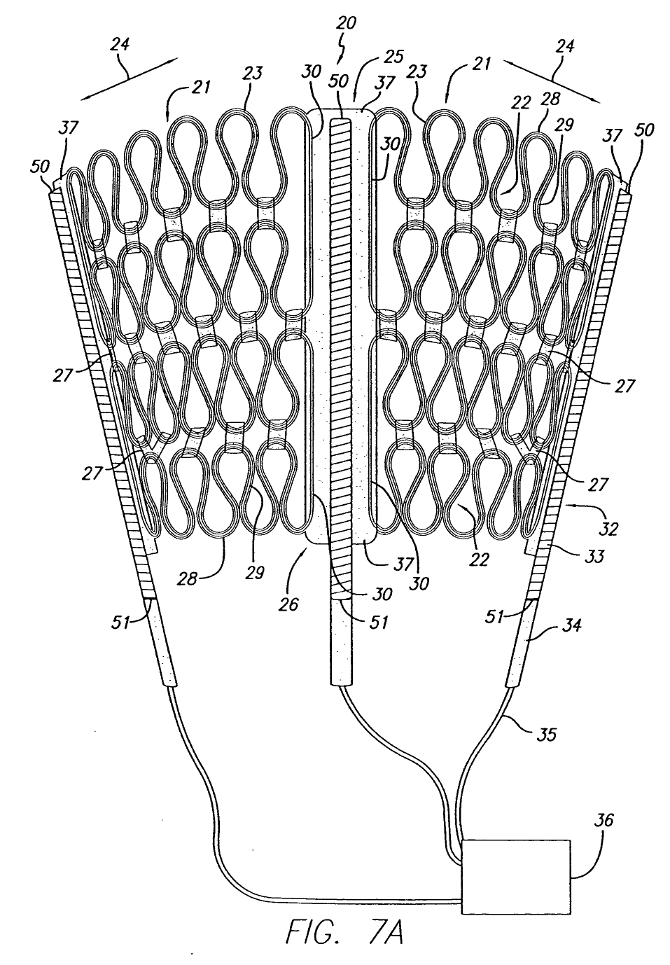

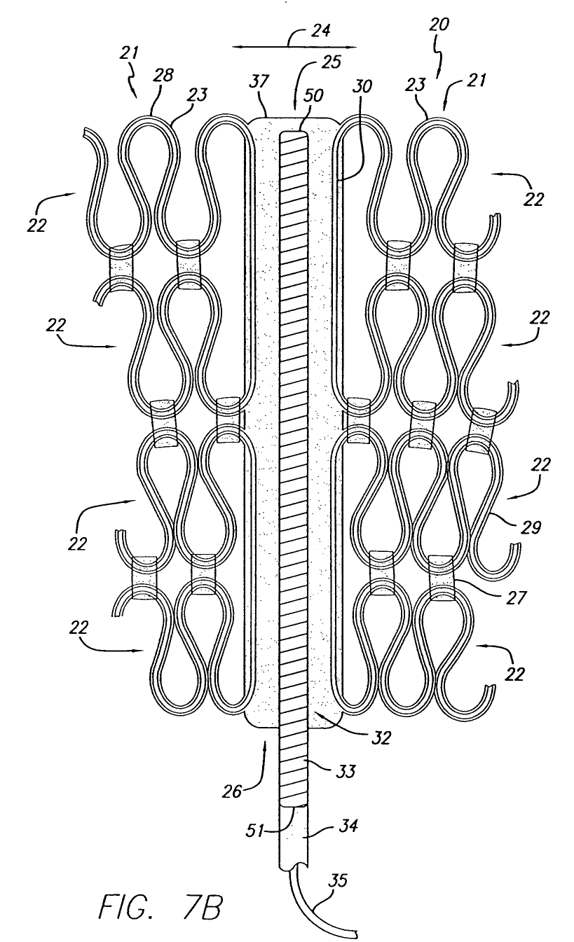

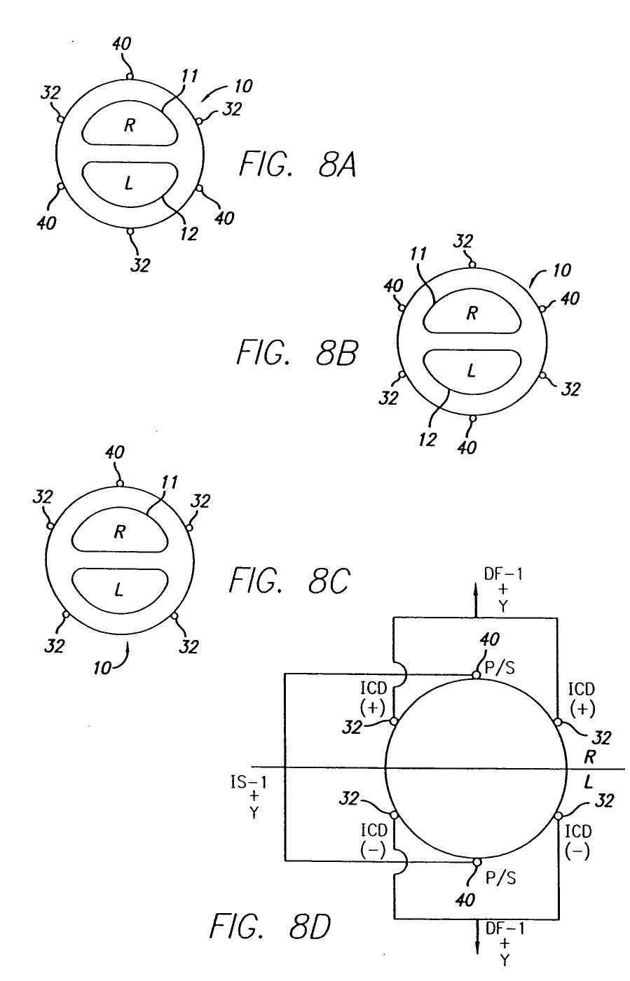

As shown in FIG. 5, the undulating strands 22 are connected to each other by grip pads 27. In the embodiments shown in FIG. 5, adjacent undulating strands are connected by one or more grip pads attached at the apex 28 of the spring elements 23. The number of grip pads between adjacent undulating strands is a matter of choice and can range from one grip pad between adjacent undulating strands, to one grip pad for every apex on the undulating strand. Importantly, the grip pads should be positioned in order to maintain flexibility of the cardiac harness 20 without sacrificing the objectives of maintaining the spacing between adjacent undulating strands to prevent overlap and to enhance the frictional engagement between the grip pads and the epicardial surface of the heart. Further, while it is desirable to have the grip pads attached at the apex of the spring elements, the invention is not so limited. The grip pads 27 can be attached anywhere along the length of the spring elements, including the sides 29. Further, the shape of the grip pads 27, as shown in FIG. 5, can vary to suit a particular purpose. For example, grip pad 27 can be attached to the apex 28 of one undulating strand 22, and be attached to two apices on an adjacent undulating strand (see FIG. 7). As shown in FIG. 5, all of the apices point toward each other, and are said to be “out-of-phase.” If the apices of the undulations were aligned, they would be “in-phase.” The apices are all out-of-phase since the number of spring elements in each undulating strand is the same, however, the invention contemplates that the number of spring elements in each undulating strand may vary since the heart is tapered from its base near the top to its apex 13 at the bottom. Thus, there would be more spring elements and a longer undulating strand per panel at the top or base of the cardiac harness than at the bottom of the cardiac harness near the apex of the heart. Accordingly, the cardiac harness would be tapered from the relatively wide base to a relatively narrow bottom toward the apex of the heart, and this would affect the alignment of the apices of the spring elements, and hence the ability of the grip pads 27 to align perfectly and attach to adjacent apices of the spring elements. A further disclosure and embodiments relating to the undulating strands and the attachment means in the form of grip pads is found in co-pending and co-assigned U.S. Ser. No. 60/486,062 filed Jul. 10, 2003, the entire contents of which are incorporated herein by reference. While the connections between adjacent undulating strands 22 is preferably grip pads 27, in an alternative embodiment (not shown) the undulating strands are connected by interconnecting elements made of the same material as the strands. The interconnecting elements can be straight or curved as shown in FIGS. 8A-8B of commonly owned U.S. Pat. No. 6,612,979 B2, the entire contents of which is incorporated by reference herein.

It is preferred that the undulating strands 22 be continuous as shown in FIG. 5. For example, every pair of adjacent undulating strands are connected by bar arm 30. It is preferred that the bar arms form part of a continuous wire that is bent to form the undulating strands, and then welded at its ends along the bar arm. The weld is not shown in FIG. 5, but can be by any conventional method such as laser welding, fusion bonding, or conventional welding. The type of wire used to form the undulating strands may have a bearing on the method of attaching the ends of the wire used to form the undulating trand. For example, it is preferred that the undulating strands be made out of a nickel-titanium alloy, such as Nitinol, which may lose some of its superelastic or shape memory properties if exposed to high heat during conventional welding.

Associated with the cardiac harness of the present invention is a cardiac rhythm management device as previously disclosed. Thus, associated with the cardiac harness as shown in FIG. 5, are one or more electrodes for use in providing defibrillating shock. As can be seen immediately below, any number of factors associated with congestive heart failure can lead to fibrillation, acquiring immediate action to save the patient's life.

Diseased hearts often have several maladies. One malady that is not uncommon is irregularity in heartbeat caused by irregularities in the electrical stimulation system of the heart. For example, damage from a cardiac infarction can interrupt the electrical signal of the heart. In some instances, implantable devices, such as pacemakers, help to regulate cardiac rhythm and stimulate heart pumping. A problem with the heart's electrical system can sometimes cause the heart to fibrillate. During fibrillation, the heart does not beat normally, and sometimes does not pump adequately. A cardiac defibrillator can be used to restore the heart to normal beating. An external defibrillator typically includes a pair of electrode paddles applied to the patient's chest. The defibrillator generates an electric field between electrodes. An electric current passes through the patient's heart and stimulates the heart's electrical system to help restore the heart to regular pumping.

Sometimes a patient's heart begins fibrillating during heart surgery or other open-chest surgeries. In such instances, a special type of defibrillating device is used. An open-chest defibrillator includes special electrode paddles that are configured to be applied to the heart on opposite sides of the heart. A strong electric field is created between the paddles, and an electric current passes through the heart to defibrillate the heart and restore the heart to regular pumping.

In some patients that are especially vulnerable to fibrillation, an implantable heart defibrillation device may be used. Typically, an implantable heart defibrillation device includes an implantable cardioverter defibrillator (ICD) or a cardiac resynchronization therapy device (CRT-D) which usually has only one electrode positioned in the right ventricle, and the return electrode is the defibrillator housing itself, typically implanted in the pectoral region. Alternatively, an implantable device includes two or more electrodes mounted directly on, in or adjacent the heart wall. If the patient's heart begins fibrillating, these electrodes will generate an electric field therebetween in a manner similar to the other defibrillators discussed above.

Testing has indicated that when defibrillating electrodes are applied external to a heart that is surrounded by a device made of electrically conductive material, at least some of the electrical current disbursed by the electrodes is conducted around the heart by the conductive material, rather than through the heart. Thus, the efficacy of defibrillation is reduced. Accordingly, the present invention includes several cardiac harness embodiments that enable defibrillation of the heart and other embodiments disclose means for defibrillating, resynchronization, left ventricular pacing, right ventricular pacing, and biventricular pacing/sensing.

In further keeping with the invention, the cardiac harness 20 includes a pair of leads 31 having conductive electrode portions 32 that are spaced apart and which separate panels 21. As shown in FIG. 5, the electrodes are formed of a conductive coil wire 33 that is wrapped around a non-conductive member 34, preferably in a helical manner. A conductive wire 35 is attached to the coil wire and to a power source 36. As used herein, the power source 36 can include any of the following, depending upon the particular application of the electrode: a pulse generator; an implantable cardioverter/defibrillator; a pacemaker; and an implantable cardioverter/defibrillator coupled with a pacemaker. In the embodiment shown in FIG. 5, the electrodes are configured to deliver an electrical shock, via the conductive wire and the power source, to the epicardial surface of the heart so that the electrical shock passes through the myocardium. Even though the electrodes are spaced so that they would be about 180° apart around the circumference of the heart in the embodiment shown, they are not so limited. In other words, the electrodes can be spaced so that they are about 45° apart, 60° apart, 90° apart, 120° apart, or any arbitrary arc length spacing, or, for that matter, essentially any arc length apart around the circumference of the heart in order to deliver an appropriate electrical shock. As previously described, it may become necessary to defibrillate the heart and the electrodes 32 are configured to deliver an appropriate electrical shock to defibrillate the heart.

Still referring to FIG. 5, the electrodes 32 are attached to the cardiac harness 20, and more particularly to the undulating strands 22, by a dielectric material 37. The dielectric material insulates the electrodes from the cardiac harness so that electrical current does not pass from the electrode to the harness thereby undesirably shunting current away from the heart for defibrillation. Preferably, the dielectric material covers the undulating strands 22 and covers at least a portion of the electrodes 32. In the FIG. 5 embodiment, the middle panel undulating strands are covered with the dielectric material while the right and left side panels are bare metal. While it is preferred that all of the undulating strands of the panels be coated with the dielectric material, thereby insulating the harness from the electric shock delivered by the electrodes, some or all of the undulating strands can be bare metal used to deliver the electrical shock to the epicardial surface of the heart for defibrillation or for pacing.

As will be described in more detail, the electrodes 32 have a conductive discharge first surface 38 that is intended to be proximate to or in direct contact with the epicardial surface of the heart, and a conductive discharge second surface 39 that is opposite to the first surface and faces away from the heart surface. As used herein, the term “proximate” is intended to mean that the electrode is positioned near or in direct contact with the outer surface of the heart, such as the epicardial surface of the heart. The first surface and second surface typically will not be covered with the dielectric material 37 so that the bare metal conductive coil can transmit the electrical current from the power source (pulse generator), such as an implantable cardioverter/defibrillator (ICD or CRT-D) 36, to the epicardial surface of the heart. In an alternative embodiment, either the first or the second surface may be covered with dielectric material in order to preferentially direct the current through only one surface. Further details of the construction and use of the leads 31 and electrodes 33 of the present invention, in conjunction with the cardiac harness, will be described more fully herein.

Importantly, the dielectric material 37 used to attach the electrodes 32 to the undulating strands 22 insulates the undulating strands from any electrical current discharged through the conductive metal coils 33 of the electrodes. Further, the dielectric material in this embodiment is flexible so that the electrodes can serve as a seam or hinge to fold the cardiac harness 20 into a lower profile for minimally invasive delivery. Thus, as will be described in more detail (see FIGS. 13 and 14), the cardiac harness can be folded along its length, along the length of the electrodes, in order to reduce the profile for intercostal delivery, for example through the rib cage or other area typically used for minimally invasive surgery for accessing the heart. Minimally invasive approaches involving the heart typically are made through subxiphoid, subcostal or intercostal incisions. When the cardiac harness is folded, it can be reduced into a circular or a more or less oval shape, both of which promote minimally invasive procedures.

In further keeping with the invention, cross sectional views of the leads 31 and the electrode portion 32 are shown in FIGS. 5B, 5C, and 5D. As shown in FIG. 5B, the electrode 32 has the coil wire 33 wrapped around the non-conducting member 34 in a helical pattern. The dielectric material 37 provides a spaced connection between the electrode and the bar arms 30 at the ends of the undulating strands 22. The electrodes do not touch or overlap with the bar arms or any portion of the undulating strands. Instead, the dielectric material provides the attachment means between the electrodes and the bar arms of the undulating strands. Thus, the dielectric material 37 not only acts as an insulating non-conductive material, but also provides attachment means between the undulating strands and the electrodes. Because the dielectric material 37 is relatively thin at the attachment points, it is highly flexible and permits the electrodes to be flexible along with the cardiac harness panels 21, which will expand and contract as the heart beats as previously described.

Referring to FIG. 5C, the non-conductive member 34 extends beyond the coil wire 33 for a distance. The non-conductive member preferably is made from the same material as the dielectric material 37, typically a silicone rubber or similar material. While it is preferred that the dielectric material be made from silicone rubber, or a similar material, it also can be made from Parylene™ (Union Carbide), polyurethanes, PTFE, TFE, and ePTFE. As can be seen, the non-conductive member provides support for the dielectric material to attach the bar arms 30 of the undulating strands 22 in order to connect the strands to the electrode 32. A conductive wire 35 extends through the non-conducting member and attaches to the proximal end of the coil wire 33 so that when an electrical current is delivered from the power source 36 through conductive wire 35, the electrode coil 33 will be energized. The conductive wire 35 is also covered by non-conducting material 34. Referring to FIG. 5D, it can be seen that the non-conductive member 34 continues to extend beyond the bottom (apex) of the cardiac harness and that conductive wire 35 continues to extend out of the non-conductive member and into the power source 36. In the embodiment shown in FIGS. 5B-5D, the cardiac harness is insulated from the electrodes by the dielectric material 37 so that there is no shunting of electrical currents by the cardiac harness 20 from the electrical shock delivered by the electrodes during defibrillation or pacing functions.

While it is preferred that the cardiac harness 20 be comprised of undulating strands 22 made from a solid wire member, such as a superelastic or shape memory material such as Nitinol, and be insulated from the electrodes 32, it is possible to use some or all of the undulating strands to deliver the electrical shock to the epicardial surface of the heart. For example, as shown in FIG. 6A, a composite wire 45 can be used to form the undulating strands 22 and, importantly, to effectively transmit current to deliver an electrical shock to the epicardial surface of the heart. The composite wire 45 includes a current conducting wire 47 made from, for example silver (Ag), and which is covered by a Nitinol tube 46. In order to improve the surface conductivity of the outer Nitinol tube 46, a highly conductive coating is placed on the Nitinol tube. For example, the Nitinol tube can be covered with a deposition layer of platinum (Pt) or platinum-iridium (Pt—Ir), or an equivalent material including iridium oxide (IROX). The composite wire, so constructed, will have superior mechanical performance to expand and contract due to the Nitinol tubing, and also will have improved electrical properties resulting from the current conducting wire 47 and improved electrolytic/electrochemical properties via the surface layer of platinum-iridium. Thus, if some portion or all of the undulating strands 22 are made from a composite wire 45, the cardiac harness 20 will be capable of delivering a defibrillating shock on selected portions of the heart via the undulating strands and will also function to impart compressive forces as previously described.

In contrast to the current conducting undulating strands of FIG. 6A, are the non-conducting insulated undulating strands 22 as shown by cross sectional view FIG. 6B. As previously described, some or all of the undulating strands 22 can be covered with dielectric material 37 in order to insulate the strands from the electrical current delivered through the electrodes while delivering shock on the epicardial surface of the heart. Thus, as shown in FIG. 6B, the undulating strands 22 are covered by dielectric material 37 to provide insulation from the electrical shock delivered by the electrodes 32, yet maintain the flexibility and the expansive properties of the undulating strands.

An important aspect of the invention is to provide a cardiac harness 20 that can be implanted minimally invasively and be attached to the epicardial surface of the heart, without requiring sutures, clips, screws, glue or other attachment means. Importantly, the undulating strands 22 may provide relatively high frictional engagement with the epicardial surface, depending on the cross-sectional shape of the strands. For example, in the embodiment disclosed in FIG. 6C, the cross-sectional shape of the undulating strands 22 can be circular, rectangular, triangular or for that matter, any shape that increases the frictional engagement between the undulating strands and the epicardial surface of the heart. As shown in FIG. 6C, the middle cross-section view having a flat rectangular surface (wider than tall) not only has a low profile for enhancing minimally invasive delivery of the cardiac harness, but it also has rectangular edges that may have a tendency to engage and dig into the epicardium to increase the frictional engagement with the epicardial surface of the heart. With the proper cross-sectional shape for the undulating strands, coupled with the grip pads 27 having a high frictional engagement feature, the necessity for suturing, clipping, or further attachment means to attach the cardiac harness to the epicardial surface of the heart becomes unnecessary.

In another embodiment as shown in FIGS. 7A and 7B, a different configuration for cardiac harness 20 and the electrodes 32 are shown, as compared to the FIG. 5 embodiments. In FIGS. 7A and 7B, three electrodes are shown separating the three panels 21 with undulating strands 22 extending between the electrodes. As with previous embodiments, springs 23 are formed by the undulating strands so that the undulating strands can expand and contract during the diastolic and systolic functions, and apply a compressive force during both functions. The far side panel of FIG. 7A is not shown for clarity purposes. The position of the electrodes around the circumference of the heart is a matter of choice, and in the embodiment of FIG. 7A, the electrodes can be spaced an equal distance apart at about 120°. Alternatively, it may be important to deliver the electrical shock more through the right ventricle requiring the positioning of the electrodes closer to the right ventricle than to the left ventricle. Similarly, it may be more important to deliver an electrical shock to the left ventricle as opposed to the right ventricle. Thus, positioning of electrodes, as with other embodiments, is a matter of choice.

Still referring to FIGS. 7A and 7B, in this embodiment electrodes 32 extend beyond the bottom or apex portion of the cardiac harness 20 in order to insure that the electrical shock delivered by the electrodes is delivered to the epicardial surface of the heart and including the lower portion of the heart closer to the apex 13. Thus, the electrodes 22 have a distal end 50 and a proximal end 51 where the proximal end is positioned closer to the apex 13 of the heart and the distal end is positioned closer to the base or upper portion of the heart. As used herein, distal is intended to mean further into the body and away from the attending physician, and proximal is meant to be closer to the outside of the body and closer to the attending physician. The proximal ends of the electrodes are positioned closer to the apex of the heart and provide several functions, including the ability to deliver an electrical shock closer to the apex of the heart. The electrode proximal ends also function to provide support for the cardiac harness 20 and the panels 21, and lend support not only during delivery (as will be further described herein) but in separating the panels and in gripping the epicardial surface of the heart to retain the harness on the heart without slipping.

While the FIGS. 7A and 7B embodiments show electrodes 32 separating three panels 21 of the cardiac panel 20, more or fewer electrodes and panels can be provided to suit a particular application. For example, in one preferred embodiment, four electrodes 32 separate four panels 21, so that two of the electrodes can be positioned on opposite sides of the left ventricle and two of the electrodes can be positioned on opposite sides of the right ventricle. In this embodiment, preferably all four electrodes would be used, with a first set of two electrodes on opposite sides of the right ventricle acting as one (common) electrode and a second set of two electrodes on opposite sides of the left ventricle acting as the opposite (common) electrode. Alternatively, two of the electrodes can be activated while the other two electrodes act as dummy electrodes in that they would not be activated unless necessary.

At present, commercially available implantable cardioverter/defibrillators (ICD's) are capable of delivering approximately thirty to forty joules in order to defibrillate the heart. With respect to the present invention, it is preferred that the electrodes 22 of the cardiac harness 20 of the present invention deliver defibrillating shocks having less than thirty to forty joules. The commercially available ICD's can be modified to provide lower power levels to suit the present invention cardiac harness system with electrodes delivering less than thirty to forty joules of power. As a general rule, one objective of the electrode configuration is to create a uniform current density distribution throughout the myocardium. Therefore, in addition to the number of electrodes used, their size, shape, and relative positions will also all have an impact on the induced current density distribution. Thus, while one to four electrodes are preferred embodiments of the invention, five to eight electrodes also are envisioned.

In keeping with the present invention, the cardiac harness and the associated cardiac rhythm management device can be used not only for providing a defibrillating shock, but also can be used as a pacing/sensing device for treating the synchrony of both ventricles, for resynchronization, for biventricular pacing and for left ventricular pacing or right ventricular pacing. As shown in FIGS. 8A-8D, the heart 10 is shown in cross-section exposing the right ventricle 11 and the left ventricle 12. The cardiac harness 20 is mounted around the outer surface of the heart, preferably on the epicardial surface of the heart, and multiple electrodes are associated with the cardiac harness. More specifically, electrodes 32 are attached to the cardiac harness and positioned around the circumference of the heart on opposite sides of the right and left ventricles. In the event that fibrillation should occur, the electrodes will provide an electrical shock through the myocardium and the left and right ventricles in order to defibrillate the heart. Also mounted on the cardiac harness, is a pacing/sensing lead 40 that functions to monitor the heart and provide data to a pacemaker. If required, the pacemaker will provide pacing stimuli to synchronize the ventricles, and/or provide left ventricular pacing, right ventricular pacing or biventricular pacing. Thus, for example, in FIG. 8C, pairs of pacing/sensing electrodes 40 are positioned adjacent the left and right ventricle free walls and can be used to provide pacing stimuli to synchronize the ventricles, or provide left ventricular pacing, right ventricular pacing or biventriculator pacing. The use of proximal Y connectors can simplify the transition to a post-generator such as Oscor's, iLink-B15-10. The iLink-B15-10 can be used to link the right and left ventricular free-wall pace/sense electrodes 40, as shown in 8D.

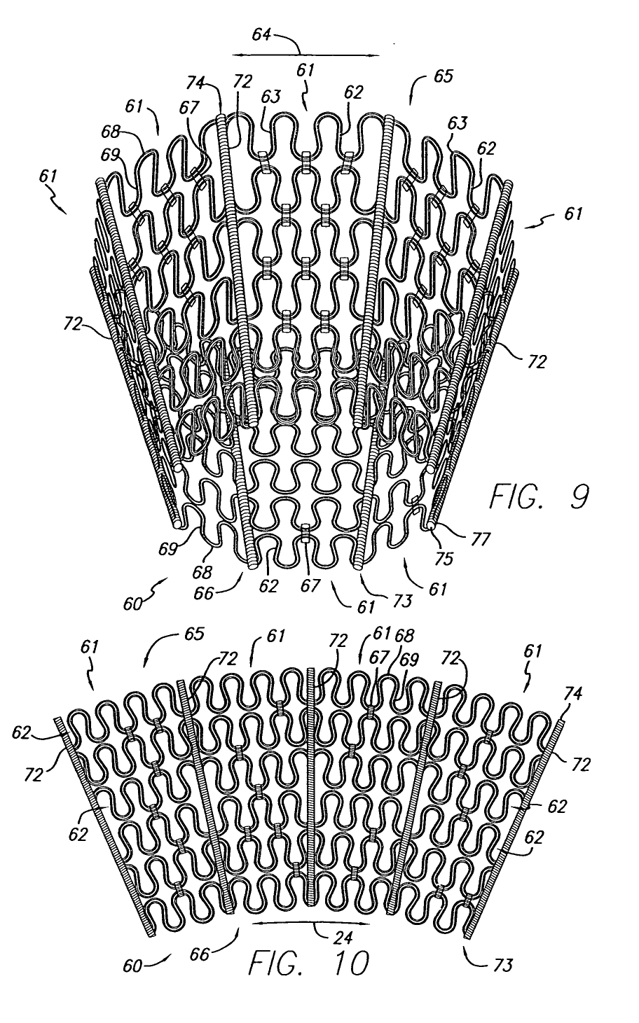

In another embodiment of the invention, as shown in FIGS. 9-14, cardiac harness 60 is similar to previously described cardiac harness 20. With respect to cardiac harness 60, it also includes panels 61 consisting of undulating strands 62. In the disclosed embodiments, the undulating strands are continuous and extend through coils as will be described. The undulating strands act as spring elements 63 as with prior embodiments that will expand and contract along directional line 64. The cardiac harness 60 includes a base or upper end 65 and an apex or lower end 66. In order to add stability to the cardiac harness 60, and to assist in maintaining the spacing between the undulating strands 62, grip pads 67 are connected to adjacent strands, preferably at the apex 68 of the springs. Alternatively, the grip pads 67 could be attached from the apex of one spring element to the side 69 of a spring element, or the grip pad could be attached from the side of one spring to the side of an adjacent spring on an adjacent undulating strand. In further keeping with the invention as shown in the FIGS. 9-14, in order to add stability and some mechanical stiffness to the cardiac harness 60, coils 62 are interwoven with the undulating strands, which together define the panels 61. The coils typically are formed of a coil of wire such as Nitinol or similar material (stainless steel, MP35N), and are highly flexible along their longitudinal length. The coils 72 have a coil apex 73 and a coil base 74 to coincide with the harness base 65 and the harness apex 66. The coils can be injected with a non-conducting material so that the undulating strands extend through gaps in the coils and through the non-conducting material. The non-conducting material also fills in the gaps which will prevent the undulating strands from touching the coils so there is no metal-to-metal touching between the undulating strands and the coils. Preferably, the non-conducting material is a dielectric material 77 that is formed of silicone rubber or equivalent material as previously described. Further, a dielectric material 78 also covers the undulating strands in the event a defibrillating shock or pacing stimuli is delivered to the heart via an external defibrillator (e.g., transthoracic) or other means.

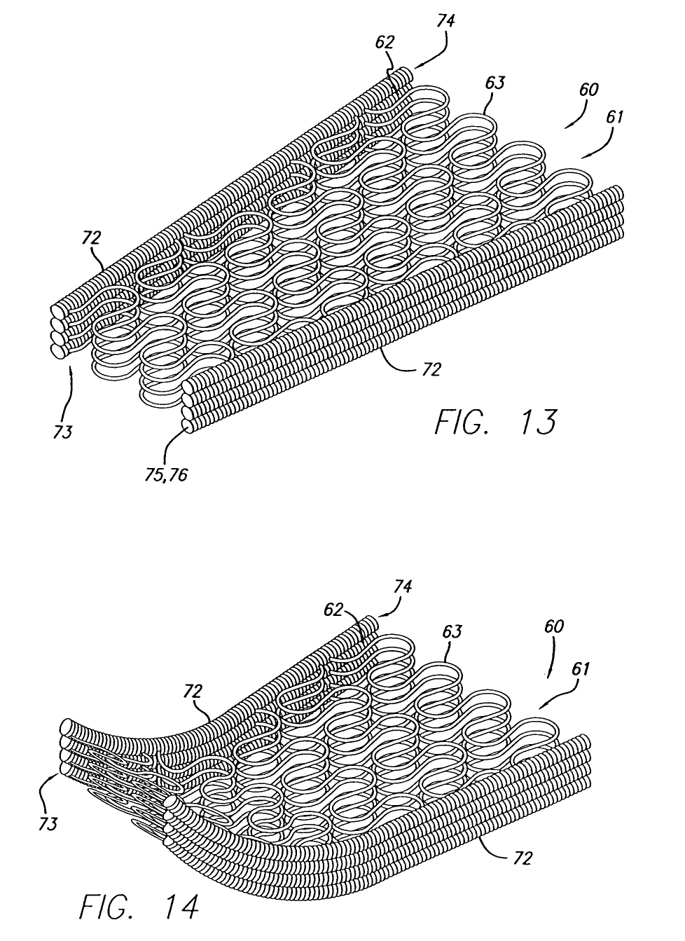

Importantly, coils 72 not only perform the function of being highly flexible and provide the attachment means between the coils and the undulating strands, but they also provide structural columns or spines that assist in deploying the harness 60 over the epicardial surface of the heart. Thus, as shown for example in FIG. 12, the cardiac harness 60 has been positioned over the heart and delivered by minimally invasive means, as will be described more fully herein. The coils 72, although highly flexible along their longitudinal length, have sufficient column strength in order to push on the apex 73 of the coils so that the base portion 74 of the coils and of the harness 65 slide over the apex of the heart and along the epicardial surface of the heart until the cardiac harness 60 is positioned over the heart, substantially as shown in FIG. 12.

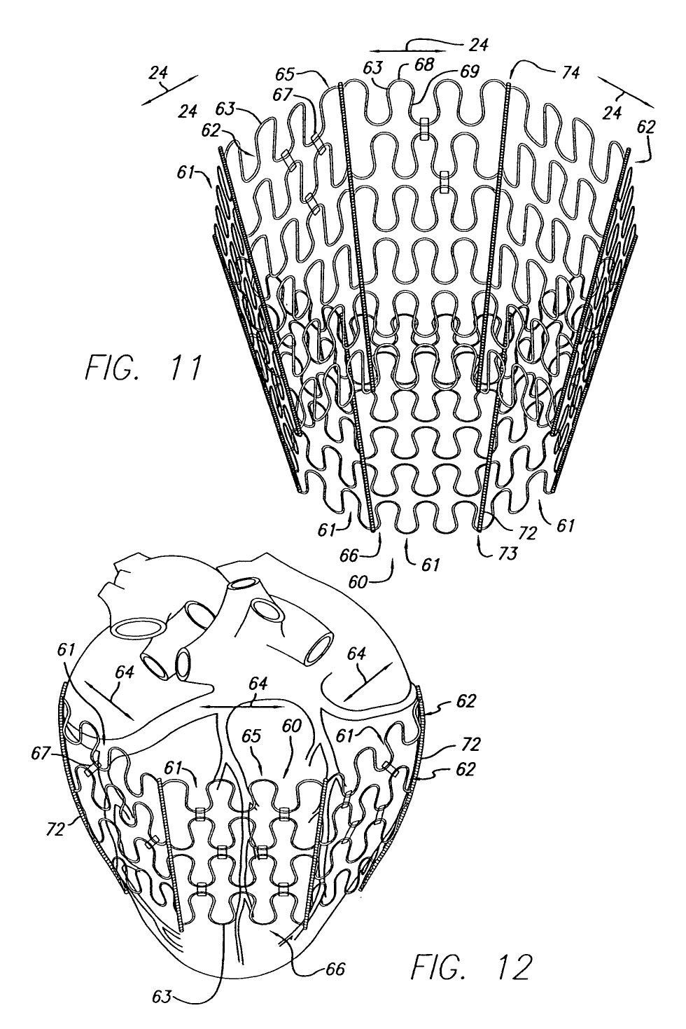

Referring to the embodiments shown in FIGS. 9 and 11, the cardiac harness 60 has multiple panels 61 and multiple coils 72. More or fewer panels and coils can be used in order to achieve a desired result. For example, eight coils are shown in FIGS. 9 and 11, while fewer coils may provide a harness with greater flexibility since the undulating strands 62 would be longer in the space between each coil. Further, the diameter of the coils can be varied in order to increase or decrease flexibility and/or column strength in order to assist in the delivery of the harness over the heart. The coils preferably have a round cross-sectional wire in the form of a tightly wound spiral or helix so that the cross-section of the coil is circular. However, the cross-sectional shape of the coil need not be circular, but may be more advantageous if it were oval, rectangular, or another shape. Thus, if coils 72 had an oval shape, where the longer axis of the oval was parallel to the circumference of the heart, the coil would flex along its longitudinal axis and still provide substantial colurnn strength to assist in delivery of the cardiac harness 60. Further, an oval-shaped coil would provide a lower profile for minimally invasive delivery. The wire cross-section also need not be round/circular, but can consist of a flat ribbon having a rectangular shape for low profile delivery. The coils also can have different shapes, for example they can be closed coils, open coils, laser-cut coils, wire-wound coils, multi-filar coils, or the coil strands themselves can be coiled (i.e., coiled coils). The electrode need not have a coil of wire, rather the electrode could be formed by a zig-zag-shaped wire (not shown) extending along the electrode. Such a design would be highly flexible and fatigue resistant yet still be capable of providing a defibrillating shock.

The cardiac harness embodiments 60 shown in FIGS. 9-12, can be folded as shown in FIGS. 13 and 14 and yet remain highly flexible for minimally invasive delivery. The coils 72 act as hinges or spines so that the cardiac harness can be folded along the longitudinal axis of the coils. The grip pads typically connecting adjacent undulating strands 62 have been omitted for clarity in these embodiments, however, they would be used as previously described.

In an alternative embodiment, similar to the embodiment shown in FIGS. 9-12, the cardiac harness 60 includes both coils 72 and electrodes 32. In this embodiment, as with the previously described embodiments, a series of undulating strands 22 extend between the coils and the electrodes to form panels 21. In this embodiment, for example, the coils and electrodes form hinge regions so that the panels can be folded along the longitudinal axis of the coils and electrodes for minimally invasive delivery. Further, in this embodiment, there are two coils and four electrodes, with two of the electrodes positioned adjacent the right ventricle, with the remaining two electrodes being positioned adjacent the left ventricle. The coils not only act as a hinge, but provide column strength as previously described so that the cardiac harness can be delivered minimally invasively by delivery through, for example, the intercostal space between the ribs and then pushing the harness over the heart. Likewise, the electrodes provide column strength as well, yet remain flexible along their longitudinal axis, as do the coils.

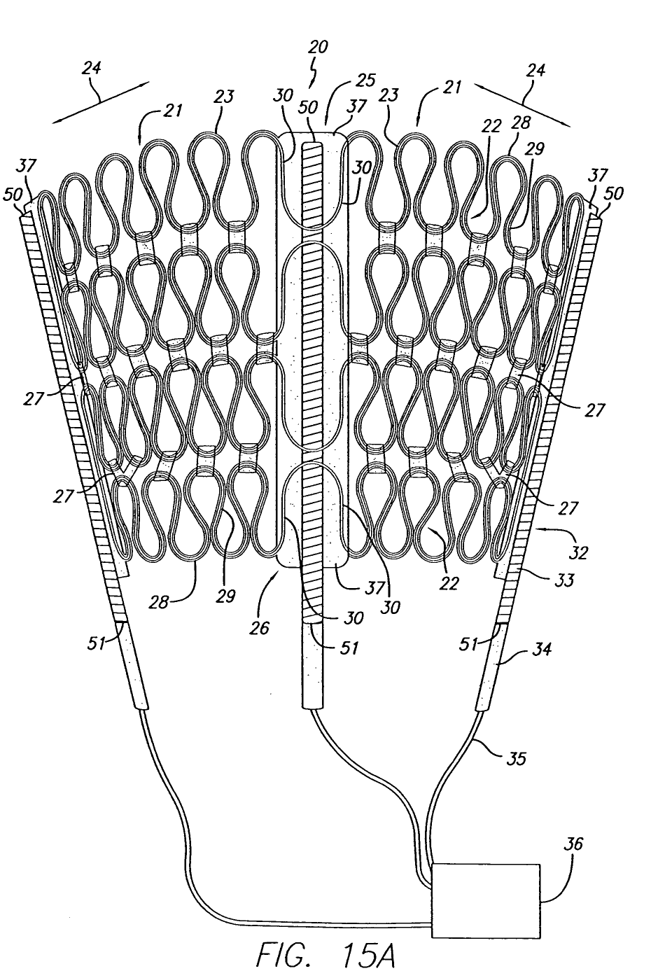

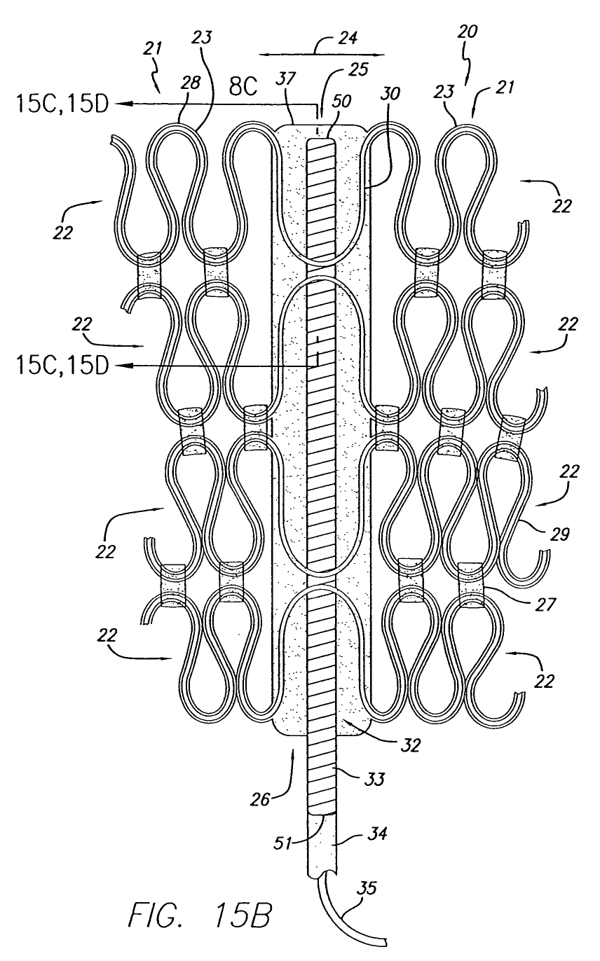

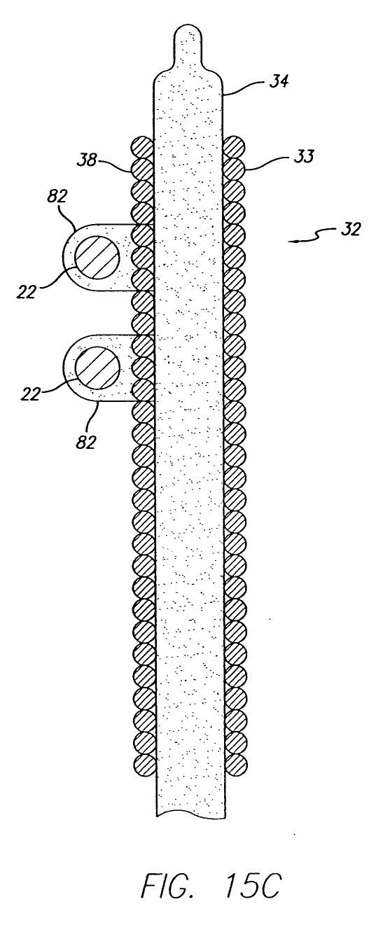

Referring to FIGS. 15A-15D, the electrodes 32 or the coils 72 can be mounted on the inner surface (touching the heart) or outer surface (away from the heart) of the cardiac harness. Thus, the cardiac harness 20 includes continuous undulating strands 22 that extend circumferentially around the heart without any interruptions. The undulating strands are interconnected by any interconnecting means, including grip pads 27, as previously described. In this embodiment, electrodes 32 or coils 72, or both, are mounted on an inner surface 80 or an outer surface 81 of the cardiac harness 20. A dielectric material 82 is molded around the electrodes or coils and around the undulating strands in order to connect the electrodes and coils to the cardiac harness. Alternatively, as shown in FIG. 15D, the electrodes 32 or coils 72 can be formed into a fastening means by forming notches 83 into the electrode (or coil) and which are configured to receive portions of the undulating strand 22. The undulating strands 22 are spaced from the coils or electrodes so that there is no overlapping/touching of metal. The notches 83 are filled with a dielectric material, preferably silicone rubber, or similar material that insulates the undulating strands of the cardiac harness from the electrodes yet provides a secure attachment means so that the electrodes or coils remain firmly attached to the undulating strands of the cardiac harness. Importantly, the electrodes 32 do not have to be in contact with the epicardial surface of the heart in order to deliver a defibrillating shock. Thus, the electrodes 32 can be mounted on the outer surface 81 of the cardiac harness, and not be in physical contact with the epicardial surface of the heart, yet still deliver a defibrillating shock as previously described.

It is to be understood that several embodiments of cardiac harnesses can be constructed and that such embodiments may have varying configurations, sizes, flexibilities, etc. Such cardiac harnesses can be constructed from many suitable materials including various metals, fabrics, plastics and braided filaments. Suitable materials also include superelastic materials and materials that exhibit shape memory properties. For example, a preferred embodiment cardiac harness is constructed of Nitinol. Shape memory dielectric materials can also be employed. Such shape memory dielectric materials can include shape memory polyurethanes or other dielectric materials such as those containing oligo(e-caprolactone) dimethacrylate and/or poly(e-caprolactone), which are available from mnemoScience.

In keeping with the invention, as shown in FIG. 16, the undulating strands 22 and 62 can be formed in many ways, including by a fixture 90. The fixture 90 has a number of stems 91 that are arranged in a pre-selected pattern that will define the shape of the undulating strands 22 and 62. The position of the stems will define the shape of the undulating strands, and determine whether the previously disclosed apex of the springs is either in-phase or out-of-phase. The shape of stems 91 will define the shape of the springs in terms of radius of curvature, or other shape, such as a keyhole shape, a U-shape, and the like. The spacing between the stems will determine the pitch and the amplitude of the undulating strands which is a matter of choice. Preferably, in one exemplary embodiment, a Nitinol wire 92 or other superelastic or shape memory wire having a 0.012 inch diameter, is woven between stems 91 in order to form the undulating strands. Other wire diameters can be used to suit a particular need and can range from about 0.007 inch to about 0.020 inch diameter. Other wire cross-section shapes are envisioned to be used with fixture 90, particularly a flat rectangular-shaped wire and an oval-shaped wire. The Nitinol wire is then heat set to impart the shape memory feature. Any free ends can be connected together by laser bonding, laser welding, or other type of similar connection consistent with the use of Nitinol, or the ends may remain free and be encapsulated in a dielectric material to keep them atraumatic, depending upon the design.

Again referring to FIG. 16, after the Nitinol wire is heat set to impart the shape memory feature, the wire is jacketed with NuSil silicone tubing (Helix Medical) having 0.029 inch outside diameter by 0.012 inch inside diameter. Thereafter, the jacketed Nitinol wire is placed in molds for transfer of liquid silicone rubber which will insulate the Nitinol wire from any electrical shock from any electrodes associated with the cardiac harness, or any other device providing a defibrillating shock to the heart. The dimensions of the silicone tubing will of course vary for different wire dimensions.

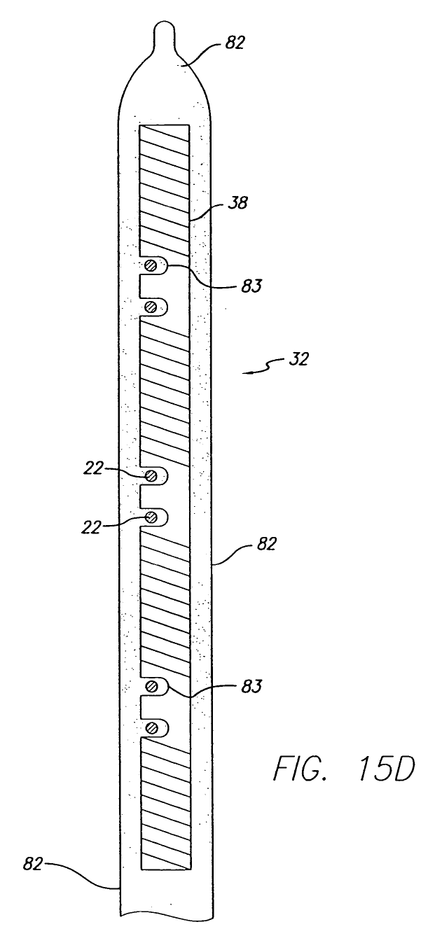

In another embodiment of the invention, shown in FIG. 17, cardiac harness 100 includes multiple panels 101 similar to those previously described. Further, undulating strands 102 form the panels and have multiple spring elements 103 that expand and contract along directional line 104, also as previously described for other embodiments. In the cardiac harness 100 shown in FIG. 17, the amplitude of the spring elements is relatively smaller than in other embodiments, and the pitch is higher, meaning there are more spring elements per unit of length relative to other embodiments. Thus, the cardiac harness 100 should generate higher bending forces as the heart expands and contracts during the diastolic and systolic cycles. In other words, the spring elements 103 of cardiac harness 100 will resist expansion, thereby imparting higher compressive forces on the wall of the heart during the diastolic function and will release these higher bending forces during the systolic function as the heart contracts. It may be important to provide undulating strands 102 that alternate in amplitude and pitch within a panel, starting at the base of the harness and extending toward the apex. For example, the pitch and amplitude of an undulating strand closer to the base or the harness may be configured to impart higher compressive forces on the epicardial surface of the heart than the undulating strands closer to the apex or the lower part of the harness. It also may be desirable to alternate the amplitude and pitch of the spring elements from one undulating strand to the next. Further, where multiple panels are provided, it may be advantageous to provide one amplitude and pitch of the spring elements of the undulating strands of one panel, and a different amplitude and pitch of the spring elements of the undulating strands of an adjacent panel. The FIG. 17 embodiment can be configured with electrodes as previously described in other embodiments, or with coils, both of which assist with the delivery of the cardiac harness by providing column support to the harness.

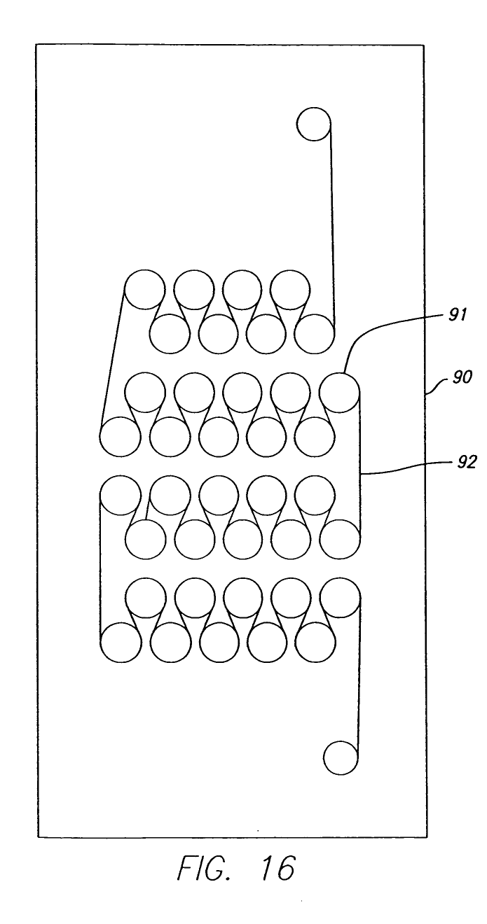

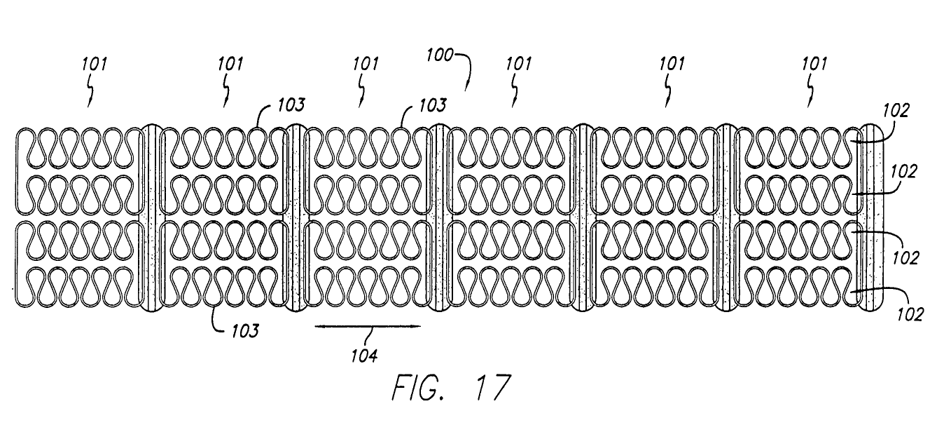

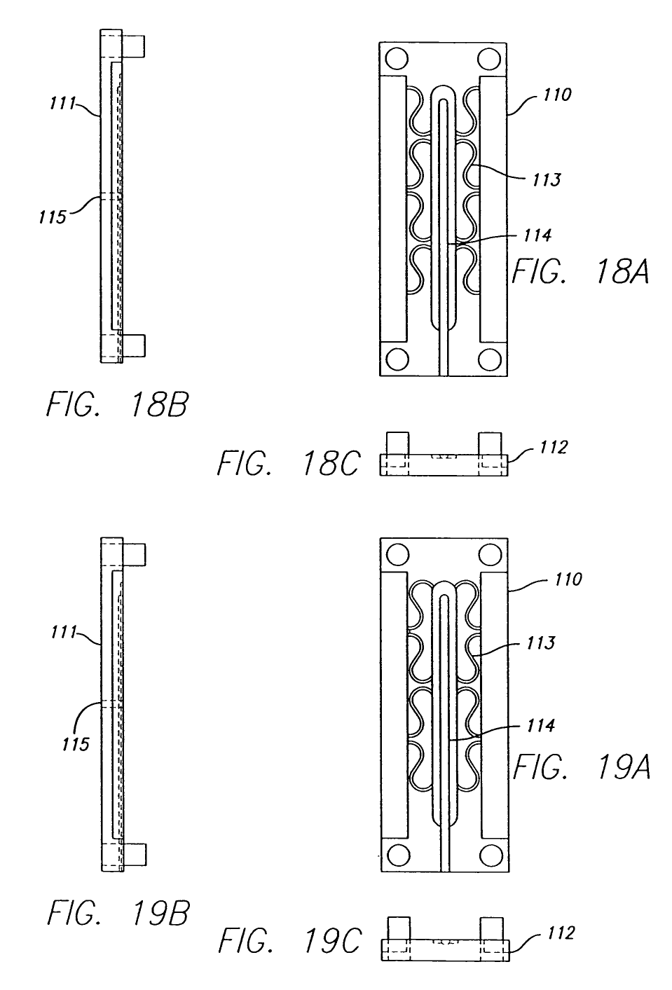

The cardiac harness of the present invention, having either electrodes or coils, can be formed using injection molding techniques as shown in FIGS. 18A-18C and 19A-19C. The molds in FIGS. 18A-18C are substantially the same as the molds shown in FIGS. 19A-19C, with the exception of the undulating pattern grooves that receive the undulating strands previously described. In referring to FIG. 18A, bottom mold 110 includes a pattern for receiving the cardiac harness and a coil or an electrode. For illustration purposes, FIG. 18B shows top mold 111 and FIG. 18C shows end view mold 112. The top mold mates with the bottom mold. As can be seen, the cardiac harness undulating strands will fit in undulating strand groove 113, which extend into coil groove 114. The previously described electrodes or coils fit into coil grooves 114. Injection port 115 is positioned midway along the mold fixtures, however, more than one injection port can be used to insure that the flow of polymer is uniform and consistent. Preferably, silicone rubber is injected into the molds so that the silicone rubber flows over the undulating strands and the electrodes or the coils. When the cardiac harness assembly is taken out of the mold, the undulating strands will be attached to the electrodes or the coils by the silicone rubber according to the pattern shown. Other patterns may be desired and the molds are easily altered to provide any pattern that ensures a secure attachment between the undulating strands and the electrodes or the coils. Importantly, the molds of FIGS. 18 and 19 can be used to inject the dielectric material or silicone rubber inside the coils and, if necessary, between the gaps in the coils in order to insure that the coils and the undulating strands are insulated from each other. The silicone rubber fills the inside of the coils, extrudes through the gaps in the coils, and forms a skin on the inner and outer surface of the coil. This skin is selectively removed (as will be described) to expose portions of the electrode coils so that they can conduct current as described. Further, it is desired that the coils and the undulating strands do not overlap or touch in order to reduce any frictional engagement between the metallic coils and the metallic undulating strands. In order to increase the frictional engagement between the cardiac harness and the epicardial surface of the heart, small projections (not shown) can be molded along the surface of the coils that will contact the epicardial surface. As previously described with respect to the grip pads, these small projections, preferably formed of silicone rubber, will engage the epicardial surface of the heart and increase the frictional engagement between the coils and the surface of the heart in order to secure the harness to the heart without the use of sutures, clips, or other mechanical attachment means.

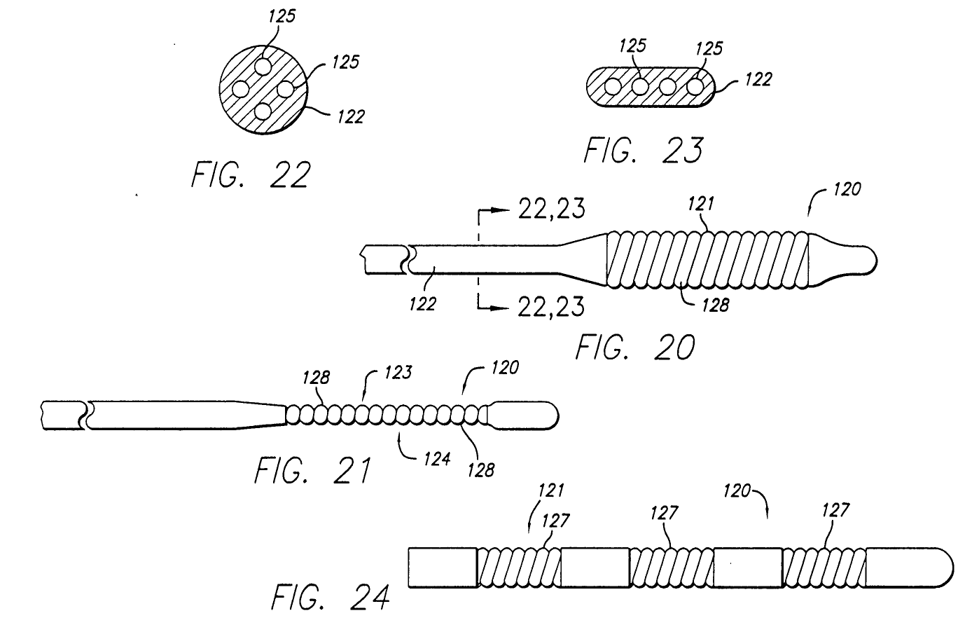

In further keeping with the invention, as shown in FIGS. 20-23, a portion of a lead having an electrode 120 is shown in the form of a conductive coil 121. The coil can be formed of any suitable wire that is conductive so that an electrical shock can be transmitted through the electrode and through the myocardium of the heart. In this embodiment, the coil wire is wrapped around a dielectric material 122 in a helical configuration, however, a spiral wrap or other configuration is possible as long as the coil has superior fatigue resistance and longitudinal flexibility. Importantly, conductive coils 121 have high fatigue resistance which is necessary since the coil is on or near the surface of the beating heart so that the coil is constantly flexing along its longitudinal length in response to heart expansion and contraction. The cross-section of the wire preferably is round or circular, however, it also can be oval shaped or flat (rectangular) in order to reduce the profile of the electrode for minimally invasive delivery. A circular, oval or flat wire will have a relatively high fatigue resistance as well as a relatively low profile for delivery purposes. Also, a flat wire coil is highly flexible along the longitudinal axis and it has a relatively high surface area for delivering an electrical shock. The electrode 120 has a first surface 123 and a second surface 124. The first surface 123 will be proximate the epicardial surface of the heart, or other portions of the heart, while the second surface will be opposite the first surface and away from the epicardial surface of the heart. A conductive wire (not shown) extends through the dielectric material 122 and attaches to the coil wire 121 at one or more locations along the coil or coils, and the conductive wire is connected to a power source (e.g., an ICD) at its other end. As shown in FIG. 22, the cross-section of the electrode 120 can be circular, or as shown in FIG. 23, can be oval for reduced profile for minimally invasive delivery. Other cross-sectional shapes for electrode 120 are available depending upon the particular need. All of these cross-sectional shapes will have relatively high fatigue resistance. As shown in FIGS. 22 and 23, multiple lumens 125 can be provided to carry one or more conductive wires from the electrode to the power source (pulse generator, ICD, CRT-D, pacemaker, etc.). The lumens also can carry sensing wires that transmit data from a sensor on or in the heart to a pacemaker so that the heart can be monitored. Further, the lumens 125 can be used for other purposes such as drug delivery (therapeutic drugs, steroids, etc.), dye injection for visibility under fluoroscopy, carrying a guide wire (not shown) or a stylet to facilitate delivery of the electrodes and the harness, or for other purposes. The lumens 125 can be used to carry a guide wire (not shown) or a stylet in such a way that the column stiffness of the coil is increased by the guide wire or stylet, or in a manner that will vary the column stiffness as required. By varying the column stiffness of the coils with a guide wire or a stylet in lumens 125, the ability to push the cardiac harness over the heart (as will be described) will be enhanced. The guide wires or stylets also can be used, to some extent, to steer the coils and hence the cardiac harness during delivery and implantation over the heart. The guide wire or stylet in lumens 125 can be removed after the cardiac harness is implanted so that the coils (electrodes) become more flexible and atraumatic.

In keeping with the invention, as shown in FIGS. 20-23, the electrode 120 not only provides an electrical conduit for use in defibrillation, but also has sufficient column strength when attached to the cardiac harness to assist in the delivery of the harness by minimally invasive means. As will be further described, the coils 121 provide a highly flexible electrode along its longitudinal length, and also provide a substantial amount of column strength when coupled with a cardiac harness to assist in the delivery of the harness.

In further keeping with the invention of FIGS. 20-23, a dielectric material such as silicone rubber 126 can be used to coat electrodes 120. During the molding process (previously described), when the electrode 120 is attached to the cardiac harness, silicone rubber 126 will coat the entire electrode 120. Soda blasting (or other known material removal process) can be used to remove portions of the silicone rubber skin from the coils 121 in order to expose first surface 123 and second surface 124 (or portions of those surfaces) so that the bare metal coil is exposed to the epicardial surface of the heart. Preferably, the silicone rubber is removed from both the first surface and the second surface, however, it also may be advantageous to remove the silicone rubber from only the first surface, which is proximate to or in contact with the epicardial surface of the heart. The electrode 120 has a surface area 128 which essentially includes all of the bare metal surface area that is exposed and that will deliver a shock. The amount of surface area per electrode can vary greatly depending upon a particular application, however, surface areas in the range from about 50 mm² to about 600 mm² are typical. While it is possible to remove the silicone rubber from only the second surface (facing away from the heart), and leaving the first surface coated with silicone rubber, an electrical shock can still be delivered from the bare metal second surface, however, the electrical shock delivered may not be as efficient as with other embodiments. While the dimensions of the electrodes can vary widely due to the variations in the size of the heart to be treated in conjunction with the size of the cardiac harness, generally the length of the electrode ranges from about 2 cm to about 16 cm. The coil 121 has a length in the range of about 1 cm to about 12 cm. Commercially available leads having one or more electrodes are available from several sources and may be used with the cardiac harness of the present invention. Commercially available leads with one or more electrodes is available from Guidant Corporation (St. Paul, Minn.), St. Jude Medical (Minneapolis, Minn.) and Medtronic Corporation (Minneapolis, Minn.). Further examples of commercially available cardiac rhythm management devices, including defibrillation and pacing systems available for use in combination with the cardiac harness of the present invention (possibly with some modification) include, the CONTAK CD®, the INSIGNIA® Plus pacemaker and FLEXTREND® leads, and the VITALITY™ AVT® ICD and ENDOTAK RELIANCE® defibrillation leads, all available from Guidant Corporation (St. Paul, Minn.), and the InSync System available from Medtronic Corporation (Minneapolis, Minn.).

In an alternative embodiment, as shown in FIG. 24, the conductive coils 121 need not be continuous along the length of the electrode 120, but can be spatially isolated or staggered along the electrode. For example, multiple coil sections 127, similar to the coil 121 shown in FIG. 20, can be spaced along the electrode with each coil section being attached to the conductive wire so it receives electrical current from the power source. The coil sections can be from about 0.5 cm to about 2.0 cm long and be spaced from about 0.5 cm to about 4 cm apart along the electrode. The dimensions used herein are by way of example only and can vary to suit a particular application

When removing portions of the silicone rubber from the electrode 120 using soda blasting or a similar technique, it may be desirable to leave portions of the electrode masked or insulated so that the masked portion is non-conductive. By masking portions of two electrodes positioned, for example, on opposite sides of the left ventricle, it is possible to vector a shock at a desirable angle through the myocardium and ventricle. The shock will travel from the bare metal (unmasked) portion of one electrode through the myocardium and the ventricle to the bare metal (unmasked) portion of the opposing electrode at a vector angle determined by the position of the masking on the electrodes.

The cardiac rhythm management devices associated with the present invention are implantable devices that provide electrical stimulation to selected chambers of the heart in order to treat disorders of cardiac rhythm and can include pacemakers and implantable cardioverter/defibrillators and/or cardiac resynchronization therapy devices (CRT-D). A pacemaker is a cardiac rhythm management device which paces the heart with timed pacing pulses. As previously described, common conditions for which pacemakers are used is in the treatment of bradycardia (ventricular rate is too slow) and tachycardia (cardiac rhythms are too fast). As used herein, a pacemaker is any cardiac rhythm management device with a pacing functionality, regardless of any other functions it may perform such as the delivery of cardioversion or defibrillation shocks to terminate atrial or ventricular fibrillation. An important feature of the present invention is to provide a cardiac harness having the capability of providing a pacing function in order to treat the synchrony of both ventricles. To accomplish the objective, a pacemaker with associated leads and electrodes are associated with and incorporated into the cardiac harness of the present invention. The pacing/sensing electrodes, alone or in combination with defibrillating electrodes, provide treatment to synchronize the ventricles and improve cardiac function.

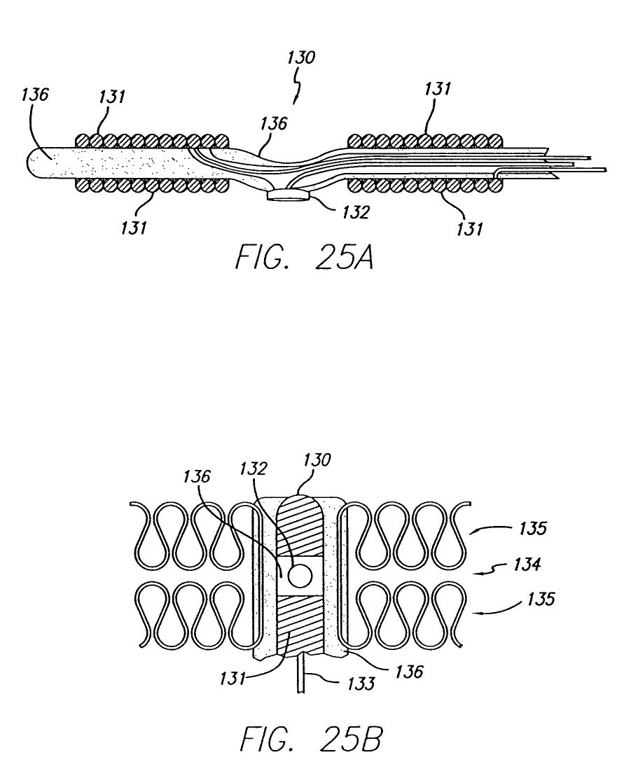

In keeping with the invention, a pacemaker and a pacing/sensing electrode are incorporated into the design of the cardiac harness. As shown in FIGS. 25A and 25B, a lead (not shown) having a defibrillator electrode 130 at its distal end, shown in partial section, not only incorporates wire coils 131 used to deliver a defibrillating electrical shock to the epicardial surface of the heart, but also incorporates a pacing/sensing electrode 132. The defibrillator electrode 130 can be attached to any cardiac harness embodiment previously described herein. In this embodiment, a non-penetrating pacing/sensing electrode 132 is combined with the defibrillating electrode 130 in order to provide data relating to heart function. More specifically, the pacing/sensing electrode 132 does not penetrate the myocardium in this embodiment, however, it may be beneficial in other embodiments for the pacing or sensing electrode to penetrate the myocardium. One advantage of a non-penetrating pacing/sensing electrode is that there is no danger of puncturing a coronary artery or causing further trauma to the epicardium or myocardium. It is also easier to design since there is no requirement of a penetration mechanism (barb or screw) on the pacing/sensing electrode. The pacing/sensing electrode 132 is in direct contact with the epicardial surface of the heart and will provide data via lead wire 133 to the pulse generator (pacemaker), which will interpret the data and provide any pacing function necessary to achieve, for example, ventricular resynchronization therapy, left ventricular pacing, right ventricular pacing, synchrony of both ventricles, and/or biventricular pacing. As shown in FIG. 25B, the pacing/sensing electrode 132 is incorporated into a portion of a cardiac harness 134, and more particularly the undulating strands 135 are attached by dielectric material 136 to the pacing/sensing electrode. As can be seen in FIGS. 25A and 25B, the wire coils 131 of the defibrillating electrode 130 are wrapped around the dielectric material 136, and the dielectric material insulates the pacing/sensing electrode 132 from both the wire coils 131 and from the undulating strands 135 of the cardiac harness. Multiple pacing/sensing electrodes 132 can be incorporated along defibrillating electrode 130, and multiple pacing and sensing electrodes can be incorporated on other electrodes associated with the cardiac harness.

In one of the preferred embodiments, multi-site pacing (as previously shown in FIGS. 8A-8D) using pacing/sensing electrodes 132 enables resynchronization therapy in order to treat the synchrony of both ventricles. Multi-site pacing allows the positioning of the pacing/sensing electrodes to provide bi-ventricular pacing or right ventricular pacing, left ventricular pacing, depending upon the patient's needs.

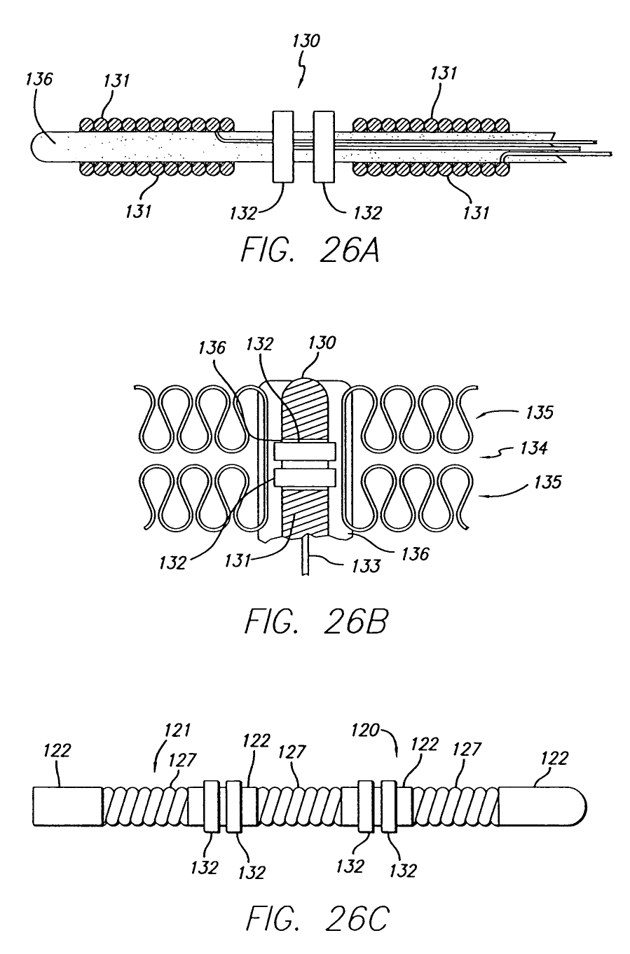

In another embodiment, shown in FIGS. 26A-26C, a defibrillating electrode is combined with pacing/sensing electrodes, for attachment to any of the cardiac harness embodiments disclosed herein. In this embodiment, the defibrillating electrode 130 is formed of wire coils 131 wrapped in a helical manner. The helical wire can be a wound wire having a single strand or a quadrafilar wire having four wires bundled together to form the coil. The wire coils 131 are wrapped around dielectric material 136 in a manner similar to that described for the embodiments in FIGS. 25A and 25B. In this embodiment, the pacing/sensing electrode 132 is in the form of a single ring for unipolar operation, and two rings for bi-polar operation. The pacing/sensing electrode rings 132 are mounted coaxially with the defibrillating electrode wire coils 131, and the conducting wires from the wire coils and the pacing/sensing ring electrode are shown extending through the dielectric material 136 and being insulated from each other. The conducting wires from the defibrillating electrode 130 and from the pacing/sensing ring electrodes 132 can be bundled into a common lead wire 133 which extends to the pulse generator (an ICD, CRT-D, and/or a pacemaker). As can be seen in FIGS. 26A-26C, the pacing/sensing electrode rings 132 have a diameter that is somewhat larger than the defibrillator electrode coils 131 in order to insure preferential contact by the electrode rings against the epicardial surface of the heart. Preferably, several pairs of pacing/sensing electrode rings (bipolar) would be positioned on the cardiac harness and be positioned to come into contact with, for example, the left ventricle free wall. Multi-site pacing allows the pacing/sensing electrode rings 132 to be used for both pacing and resynchronization concurrently. Further, the pacing/sensing electrode rings 132 also can be used in the absence of defibrillating electrodes 130. The prior disclosure relating to molding of the cardiac harness to the defibrillator electrode applies equally as well to the pacing/sensing electrode rings. The wire coil 131 and the pacing/sensing electrode rings 32 can be fabricated in several ways including by laser cutting stainless steel tubing or using highly conductive materials in wire form, such as biocompatible platinum wire. As previously disclosed, the wire coils 131 can be quadrafilar wire (platinum) for improved flexibility and conformability to the epicardial surface of the heart and be biocompatible. The surface of the pacing/sensing electrodes can vary greatly depending upon the application. As an example, in one embodiment, the surface area of the pacing/sensing electrodes are in the range from about 2 mm² to about 12 mm², however, this range can vary substantially. While the disclosed embodiments show the pacing/sensing electrodes combined with the defibrillating electrodes, the pacing/sensing electrodes can be formed separately and mounted on the cardiac harness with or without defibrillating electrodes.

The defibrillating electrode 130 as disclosed herein, can be used with commercially available pacing/sensing electrodes and leads. For example, Oscor (Model HT 52PB) endocardial/passive fixation leads can be integrated with the defibrillator electrode 130 by molding the leads into the fibrillator electrode using the same molds previously disclosed herein.

The foregoing disclosed invention incorporating cardiac rhythm management devices into the cardiac harness combines several treatment modalities that are particularly beneficial to patients suffering from congestive heart failure. The cardiac harness provides a compressive force on the heart thereby relieving wall stress, and improving cardiac function. The defibrillating and pacing/sensing electrodes associated with the cardiac harness, along with ICD's and pacemakers, provide numerous treatment options to correct for any number of maladies associated with congestive heart failure. In addition to the defibrillation function previously described, the cardiac rhythm devices can provide electrical pacing stimulation to one or more of the heart chambers to improve the coordination of atrial and/or ventricular contractions, which is referred to as resynchronization therapy. Cardiac resynchronization therapy is pacing stimulation applied to one or more heart chambers, typically the ventricles, in a manner that restores or maintains synchronized bilateral contractions of the atria and/or ventricles thereby improving pumping efficiency. Resynchronization pacing may involve pacing both ventricles in accordance with a synchronized pacing mode. For example, pacing at more than one site (multi-site pacing) at various sites on the epicardial surface of the heart to desynchronize the contraction sequence of a ventricle (or ventricles) may be therapeutic in patients with hypertrophic obstructive cardiomyopathy, where creating asynchronous contractions with multi-site pacing reduces the abnormal hyper-contractile function of the ventricle. Further, resynchronization therapy may be implemented by adding synchronized pacing to the bradycardia pacing mode where paces are delivered to one or more synchronized pacing sites in a defined time relation to one or more sensing and pacing events. An example of synchronized chamber-only pacing is left ventricle only synchronized pacing where the rate in synchronized chambers are the right and left ventricles respectively. Left-ventricle-only pacing may be advantageous where the conduction velocities within the ventricles are such that pacing only the left ventricle results in a more coordinated contraction by the ventricles than by conventional right ventricle pacing or by ventricular pacing. Further, synchronized pacing may be applied to multiple sites of a single chamber, such as the left ventricle, the right ventricle, or both ventricles. The pacemakers associated with the present invention are typically implanted subcutaneously on a patient's chest and have leads threaded to the pacing/electrodes as previously described in order to connect the pacemaker to the electrodes for sensing and pacing. The pacemakers sense intrinsic cardiac electrical activity through the electrodes disposed on the surface of the heart. Pacemakers are well known in the art and any commercially available pacemaker or combination defibrillator/pacemaker can be used in accordance with the present invention.

The cardiac harness and the associated cardiac rhythm management device system of the present invention can be designed to provide left ventricular pacing. In left heart pacing, there is an initial detection of a spontaneous signal, and upon sensing the mechanical contraction of the right and left ventricles. In a heart with normal right heart function, the right mechanical atrio-ventricular delay is monitored to provide the timing between the initial sensing of right atrial activation (known as the P-wave) and right ventricular mechanical contraction. The left heart is controlled to provide pacing which results in left ventricular mechanical contraction in a desired time relation to the right mechanical contraction, e.g., either simultaneous or just preceding the right mechanical contraction. Cardiac output is monitored by impedance measurements and left ventricular pacing is timed to maximize cardiac output. The proper positioning of the pacing/sensing electrodes disclosed herein provides the necessary sensing functions and the resulting pacing therapy associated with left ventricular pacing.

An important feature of the present invention is the minimally invasive delivery of the cardiac harness and the cardiac rhythm management device system which will be described immediately below.

Delivery of the cardiac harness 20,60, and 100 and associated electrodes and leads can be accomplished through conventional cardio-thoracic surgical techniques such as through a median sternotomy. In such a procedure, an incision is made in the pericardial sac and the cardiac harness can be advanced over the apex of the heart and along the epicardial surface of the heart simply by pushing it on by hand. The intact pericardium is over the harness and helps to hold it in place. The previously described grip pads and the compressive force of the cardiac harness on the heart provide sufficient attachment means of the cardiac harness to the epicardial surface so that sutures, clips or staples are unnecessary. Other procedures to gain access to the epicardial surface of the heart include making a slit in the pericardium and leaving it open, making a slit and later closing it, or making a small incision in the pericardium.

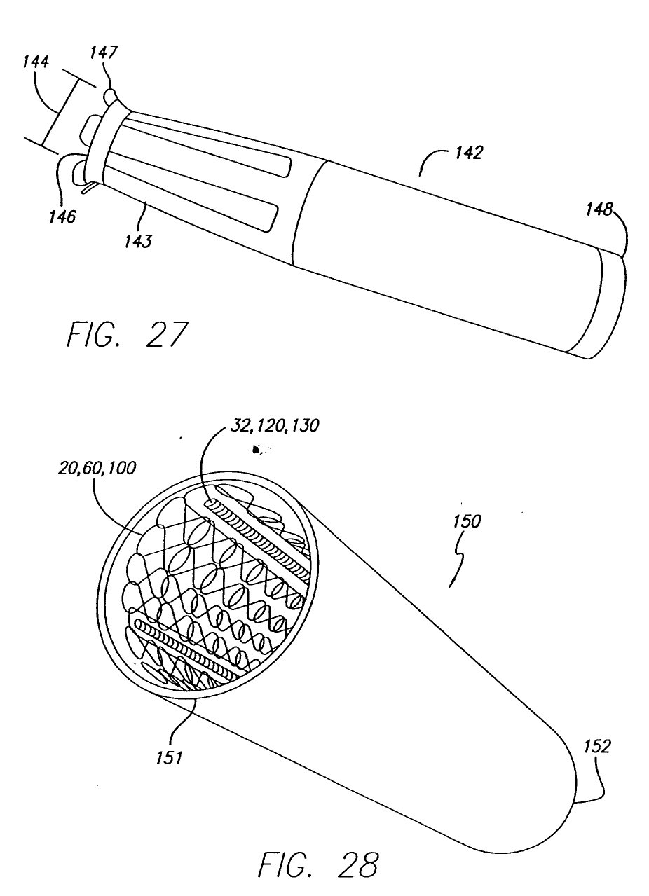

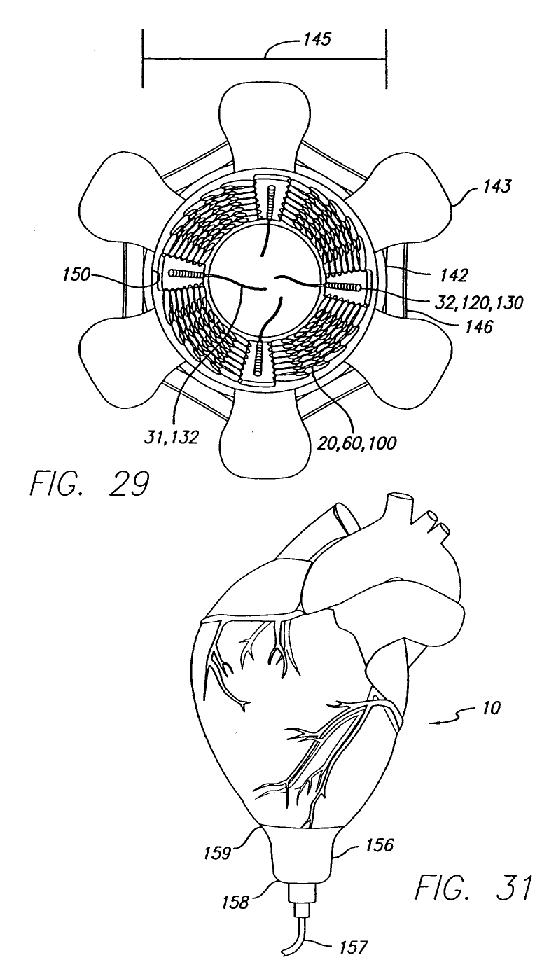

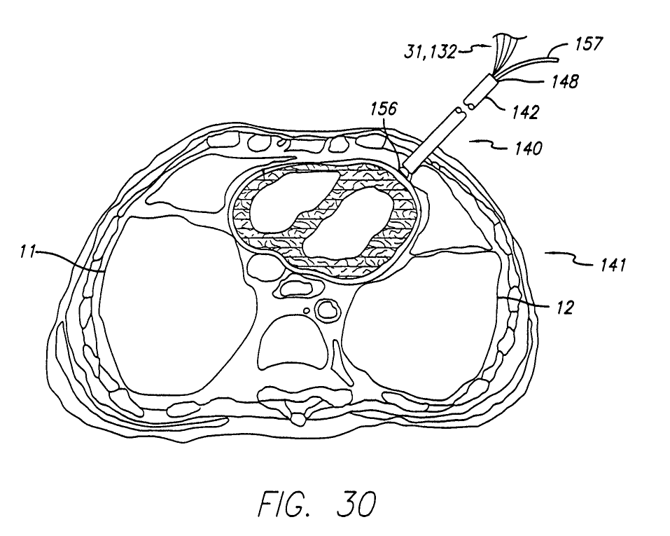

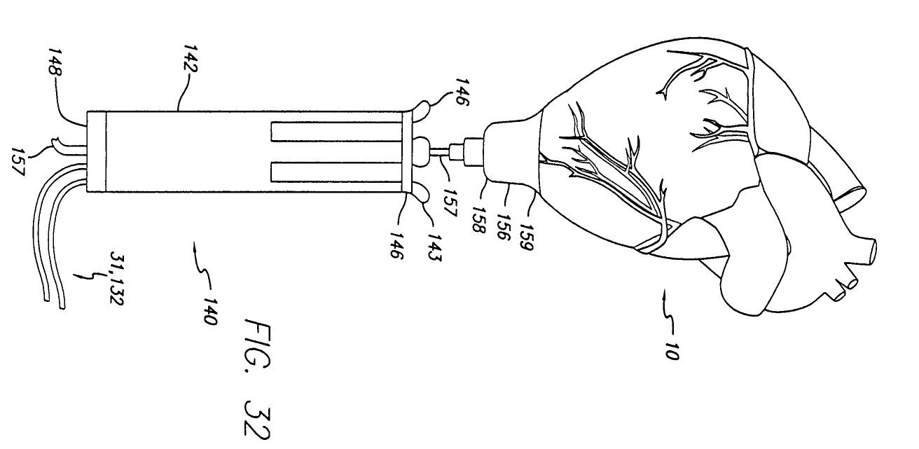

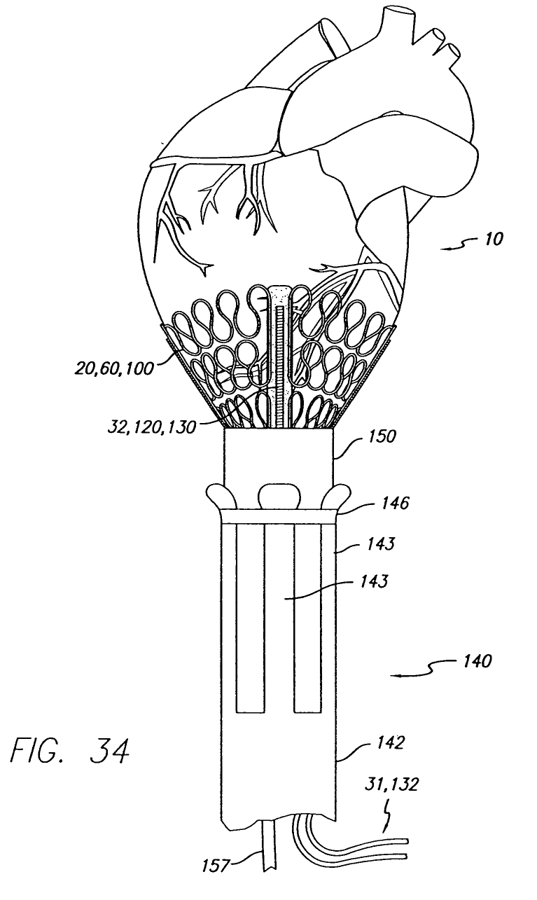

Preferably, however, the cardiac harness and associated electrodes and leads may be delivered through minimally invasive surgical access to the thoracic cavity, as illustrated in FIGS. 27-36, and more specifically as shown in FIG. 30. A delivery device 140 may be delivered into the thoracic cavity 141 between the patient's ribs to gain direct access to the heart 10. Preferably, such a minimally invasive procedure is accomplished on a beating heart, without the use of cardio-pulmonary bypass. Access to the heart can be created with conventional surgical approaches. For example, the pericardium may be opened completely or a small incision can be made in the pericardium (pericardiotomy) to allow the delivery system 140 access to the heart. The delivery system of the disclosed embodiments comprises several components as shown in FIGS. 27-36. As shown in FIG. 27, an introducer tube 142 is configured for low profile access through a patient's ribs. A number of fingers 143 are flexible and have a delivery diameter 144 as shown in FIG. 27, and an expanded diameter 145 as shown in FIG. 29. The delivery diameter is smaller than the expanded diameter. An elastic band 146 expands around the distal end 147 of the fingers and prevents the fingers from overexpanding during delivery of the cardiac harness. The distal end of the fingers is the part of the delivery device 140 that is inserted through the patient's ribs to gain direct access to the heart.

The delivery device 140 also includes a dilator tube 150 that has a distal end 151 and a proximal end 152. The cardiac harness 20,60,100 is collapsed to a low profile configuration and inserted into the distal end of the dilator tube, as shown in FIG. 28. The dilator tube has an outside diameter that is slightly smaller than the inside diameter of the introducer tube 142. As will be discussed more fully herein, the distal end 151 of the dilator tube is inserted into the proximal end 147 of the introducer tube in close sliding engagement and in a slight frictional engagement. The slidable engagement between the dilator tube and the introducer tube should be with some mild resistance, however, there should be unrestricted slidable movement between the two tubes. The distal end 151 of the dilator tube will expand the fingers 143 of the introducer tube 142 as the dilator tube is pushed distally into the introducer tube as shown in FIG. 29. In the embodiments shown in FIGS. 27-36, the cardiac harness 20,60,100 is equipped with leads (previously described) having electrodes for use in defibrillation or pacing functions.

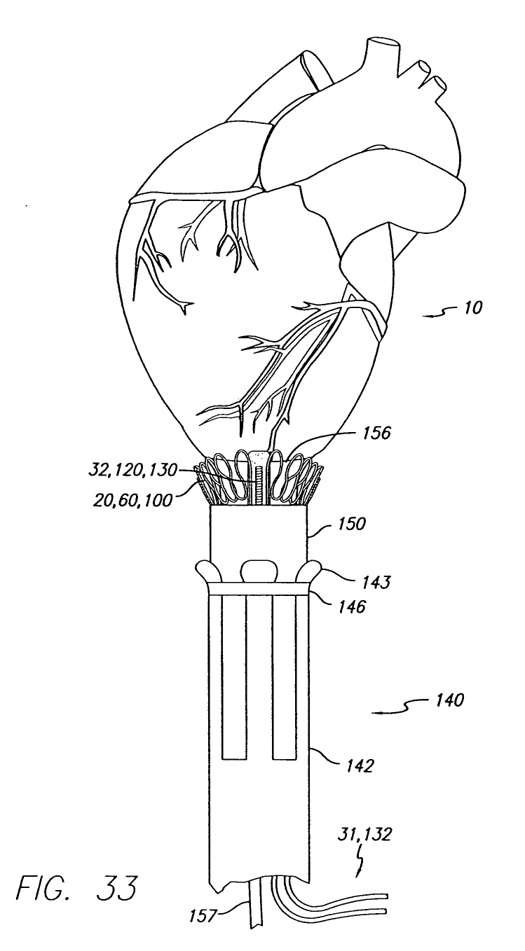

As shown in FIG. 31, the delivery system 140 also includes a releasable suction device, such as suction cup 156 at the distal end of the delivery device. The negative pressure suction cup 156 is used to hold the apex of the heart 10. Negative pressure can be applied to the suction cup using a syringe or other vacuum device commonly known in the art. A negative pressure lock can be achieved by a one-way valve stop-cock or a tubing clamp, also known in the art. The suction cup 156 is formed of a biocompatible material and is preferably stiff enough to prevent any negative pressure loss through the heart while manipulating the heart and sliding the cardiac harness 20,60,100 onto the heart. Further, the suction cup 156 can be used to lift and maneuver the heart 10 to facilitate advancement of the harness or to allow visualization and surgical manipulation of the posterior side of the heart. The suction cup has enough negative pressure to allow a slight pulling in the proximal direction away from the apex of the heart to somewhat elongate the heart (e.g., into a bullet shape) during delivery to facilitate advancing the cardiac harness over the apex and onto the base portion of the heart. After the suction cup 156 is attached to the apex of the heart and a negative pressure is drawn, the cardiac harness, which has been releasably mounted in the distal end 151 of the dilator tube 150, can be advanced distally over the heart, as will be described more fully herein.

As shown in FIG. 30, the delivery device 140, and more specifically introducer tube 142, has been advanced through the intercostal space between the patient's ribs during insertion of the introducer tube, the fingers 143 are in their delivery diameter 144, which is a low profile for ease of access through the small port made through the patient's ribs. Thereafter, the dilator tube 150, with the cardiac harness 20,60,100 mounted therein, is advanced distally through the introducer tube so that the fingers 143 are expanded until they achieve their expanded diameter 145. The suction cup 156 can be attached to the apex 13 of the heart 10 either before or after the dilator tube is advanced to spread the fingers 143 of the introducer tube 142. Preferably, the dilator tube has already expanded the fingers on the introducer tube so that there is a larger opening for the suction cup as it is advanced through the inside of a dilator tube, out of the distal end of the introducer tube, and placed in contact with the apex of the heart. Thereafter, a negative pressure is drawn allowing the suction cup to securely attach to the apex of the heart. Visualizing equipment that is commonly known in the art may be used to assist in positioning the suction cup to the apex. For example, fluoroscopy, magnetic resonance imaging (MRI), dye injection to enhance fluoroscopy, and echocardiography, and intracardiac, transesophageal, or transthoracic echo, all can be used to enhance positioning and in attaching the suction cup to the apex of the heart. After negative pressure is drawn and the suction cup is securely attached (releasably) to the apex of the heart, the heart can then be maneuvered somewhat by pulling on the tubing 157 attached to the suction cup, or by manipulating the introducer tube 142, the dilator tube 150, both in conjunction with the suction cup. As previously described, it may be advantageous to pull on the tubing 157 to allow the suction cup to pull on the apex of the heart and elongate the heart somewhat in order to facilitate sliding the harness over the epicardium.

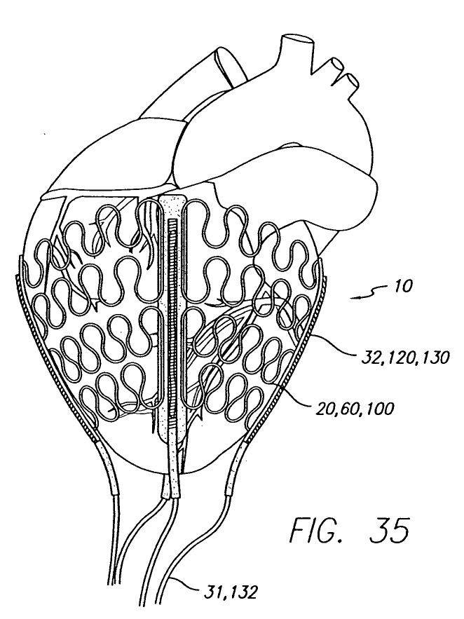

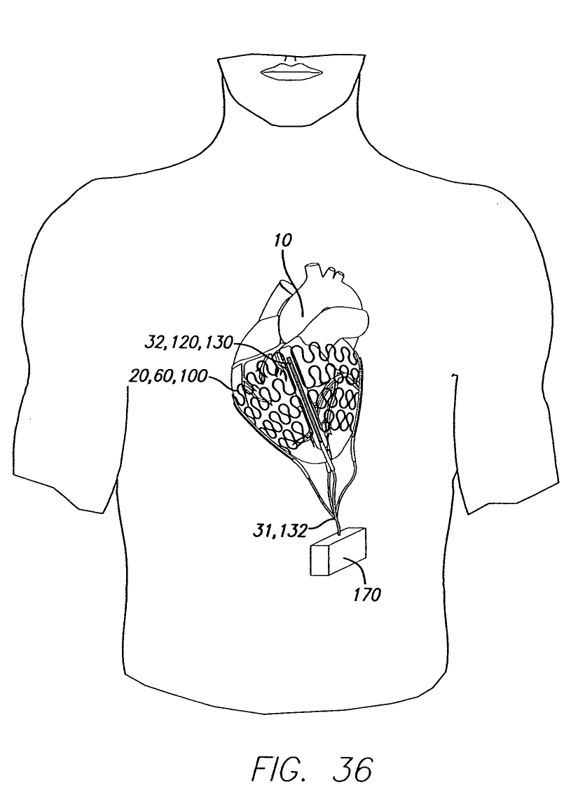

As more clearly shown in FIGS. 32-36, the cardiac harness 20,60,100 is advanced distally out of the dilator tube and over the suction cup 156. The suction cup is tapered so that the distal end of the harness slides over the narrow portion of the taper (the proximal end of the suction cup 158). The suction cup becomes wider at its distal end where it is attached to the apex of the heart, and the cardiac harness continues to slide and expand over the suction cup as it is advanced distally. As the cardiac harness continues to be advanced distally, it slides over the apex of the heart and continues to expand as it is pushed out of the dilator tube and along the epicardial surface of the heart. Since the harness and the electrodes 32,120,130 are coated with the previously described dielectric material, preferably silicone rubber, the cardiac harness should slide easily over the epicardial surface of the heart. The silicone rubber offers little resistance and the epicardial surface of the heart has sufficient fluid to allow the harness to easily slide over the wet surface of the heart. The pericardium previously has been cut so that the cardiac harness is sliding over the epicardial surface of the heart with the pericardium over the cardiac harness to help hold it onto the surface of the heart. As shown in FIGS. 35 and 36, the cardiac harness 20,60,100 has been completely advanced out of the dilator tube so that the harness covers at least a portion of the heart 10. The suction cup 156 has been withdrawn, and the introducer tube 142 and dilator tube 150 also have been withdrawn proximally from the patient. Prior to removing the introducer tube, a power source 170 (such as an ICD, CRT-D, and/or pacemaker) can be implanted by conventional means. The electrodes will be attached to the pulse generator to provide a defibrillating shock or pacing functions as previously described.

In the embodiments shown in FIGS. 27-36, the cardiac harness 20,60,100 was advanced through the dilator tube by pushing on the proximal end of the electrodes 32,120,130, on the lead wires 31,133, and on the proximal end (apex 26) of the cardiac harness. Even though the electrodes are designed to be atraumatic and longitudinally flexible, the electrodes have sufficient column strength so that pushing on the proximal ends of the electrodes assists in pushing the cardiac harness out of the dilator tube and over the epicardial surface of the heart. In one embodiment, advancement of the cardiac harness is accomplished by hand, by the physician simply pushing on the electrodes and the leads to advance the cardiac harness out of the dilator tube to slide onto the epicardial surface of the heart.

As shown in the embodiments of FIGS. 27-36, the delivery device 140, and more specifically introducer tube 142 and dilator tube 150, have a circular cross-section. It may be preferable, however, to chose other cross-sectional shapes, such as an oval cross-sectional shape for the delivery device. An oval delivery device may be more easily inserted through the intercostal space between the patient's ribs for a low profile delivery. Further, as the cardiac harness 20,60,100 is advanced out of a delivery device 140 having an oval cross-section, the harness distal end will quickly form into a more circular shape in order to assume the configuration of the epicardial surface of the heart as it is advanced distally over the heart.

In the embodiments shown in FIGS. 35 and 36, the cardiac harness 20,60,100 remains firmly attached to the epicardial surface of the heart without the need for any further attachment means, such as sutures, clips, adhesives, or staples. Further, the pericardial sac helps to enclose the harness to prevent it from shifting or sliding on the epicardial surface of the heart.

Importantly, during delivery of the cardiac harness 20,60,100, the harness itself, the electrodes 32,120,130, as well as leads 31 and 132 have sufficient column strength in order for the physician to push from the proximal end of the harness to advance it distally through the dilator tube 150. While the entire cardiac harness assembly is flexible, there is sufficient column strength, especially in the electrodes, to easily slide the cardiac harness over the epicardial surface of the heart in the manner described.

In an alternative embodiment, if the cardiac harness 20,60,100 includes coils 72, as opposed to the electrodes and leads, the harness can be delivered in the same manner as previously described with respect to FIGS. 27-36. The coils have sufficient column strength to permit the physician to push on the proximal end of the coils to advance the cardiac harness distally to slide over the apex of the heart and onto the epicardial surface.

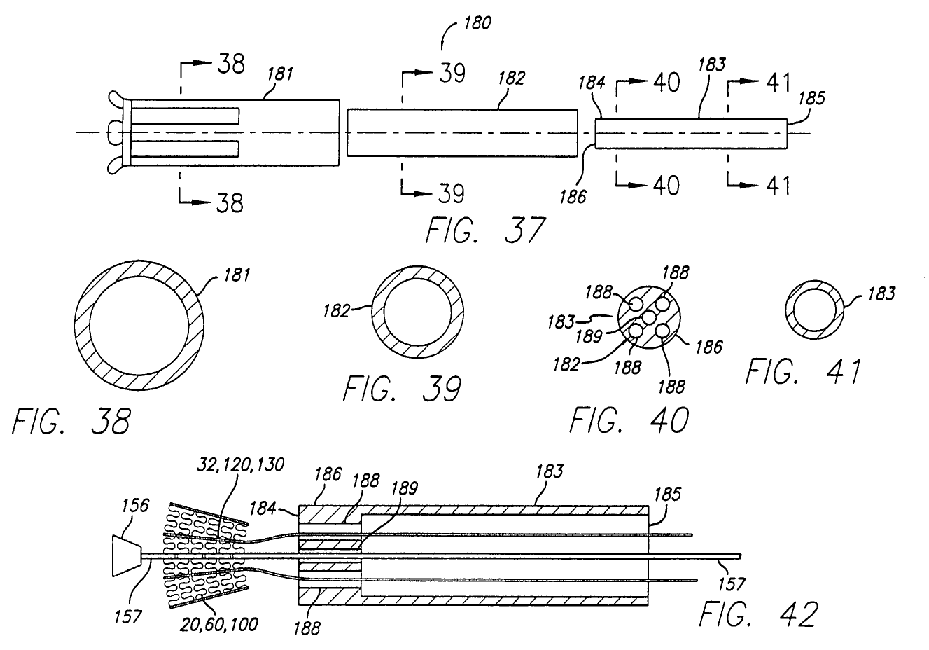

In another embodiment, delivery of the cardiac harness 20,60,100 can be by mechanical means as opposed to the hand delivery previously described. As shown in FIGS. 37-42, delivery system 180 includes an introducer tube 181 that functions the same as introducer tube 142. Also, a dilator tube 182, which is sized for slidable movement within the introducer tube, also functions the same as the previously described dilator tube 150. An ejection tube 183 is sized for slidable movement within the dilator tube, that is, the outer diameter of the ejection tube is slightly smaller than the inner diameter of the dilator tube. As shown in FIGS. 40 and 41, the ejection tube has a distal end 184 and a proximal end 185, wherein the distal end of the ejection tube has a plate that fills the entire inner diameter of the ejection tube. The plate has a number of lumens 187 for receiving leads 31,132 and for receiving the suction cup 156 and associated tubing 157. Thus, lumens 188 are sized for receiving leads 31,132 therethrough, while lumen 189 is sized for receiving suction cup 156 and the associated tubing 157. The number of lumens 188 in plate 186 will be defined by the number of leads 31,132 associated with the cardiac harness 20,60,100. Thus, as shown in FIG. 40, there are four lumens 188 for receiving four leads therethrough, and one lumen 189 for receiving the suction cup 156 and tubing 157 therethrough. The leads and the tubing 157 extend proximally out the proximal end 185 of the ejection tube. As shown in FIG. 42, the suction cup and cardiac harness are on the left side of the schematic, and the ejection tube 183 is on the right hand side of the schematic. For clarity, the dilator tube and the introducer tube have been omitted, however, in practice the cardiac harness would be mounted in the dilator tube, and the dilator tube would extend into the introducer tube, while the ejection tube would extend into the dilator tube. As can be seen in FIG. 42, the leads 31,132 extend through lumens 188, while the tubing 157 associated with the suction cup extends through lumen 189. The tubing and the leads extend proximally out of the proximal end of the ejection tube, and extend out of the patient during delivery of the harness. As previously described, after the introducer is positioned through the rib cage, and the apex of the heart is acquired by the suction cup, the harness can be advanced out of the dilator by advancing the ejection tube 183 in a distal direction toward the apex of the heart. The leads, the cardiac harness and electrodes all provide sufficient column strength to allow the plate 186 to impart a pushing force against the cardiac harness to advance it distally over the heart as previously described. After the cardiac harness is pushed over the epicardial surface of the heart, the ejection tube can be withdrawn proximally so that the tubing 157 and the leads 31,132 slide through lumens 189,188 respectively. The ejection tube 183 continues to be withdrawn proximally so that the proximal end of the leads and the proximal end of tubing 157 are pulled through the distal end 184 of the ejection tube so that the ejection tube is clear of the leads and the tubing.

As with the previous embodiment, suitable materials for the delivery system 140,180 can include the class of polymers typically used and approved for biocompatible use within the body. Preferably, the tubing associated with delivery systems 140 and 180 are rigid, however, they can be formed of a more flexible material. Further, the delivery systems 140,180 can be curved rather than straight, or can have a flexible joint in order to more appropriately maneuver the cardiac harness 20,60,100 over the epicardial surface of the heart during delivery. Further, the tubing associated with delivery systems 140,180 can be coated with a lubricious material to facilitate relative movement between the tubes. Lubricious materials commonly known in the art such as Teflon™ can be used to enhance slidable movement between the tubes.

The present invention includes a passive restraint device consisting of a wireform cardiac harness delivered through a mini-thoracotomy using a delivery system. As previously disclosed, defibrillation electrodes/leads are attached directly onto the cardiac harness. There is a need to provide the cardiac harness in combination with epicardial pace/sense electrodes to provide optimal Cardiac Resynchronization Therapy (CRT) in patients with inter- and intra-ventricular contraction dyssynchrony. While the pace/sense electrodes could be integrated into fixed positions on the harness, there is a benefit to being able to adjust the position of the pace/sense electrodes relative to the harness once on the heart. While the harness configured with integrated pace/sense electrodes could be moved to some degree in an attempt to optimize the electrode position, it is assumed that the harness is deployed into an optimal position for passive restraint and that it would be undesirable to alter that position. The benefit of adjusting the pace/sense electrode position is largely related to where the electrodes are positioned once the harness is deployed. The pace/sense electrodes may be located over a tissue region where there is insufficient sensing or pacing ability (e.g., over fat, ischemic, fibrotic, or necrotic tissue), or where there is a sub-optimal resynchronization effect. Besides sensing and pacing for CRT applications, there may be benefit to altering the placement of one or more pace/sense electrodes relative to the harness for bradycardic pacing (e.g., for backup VVI pacing, or for chronic pacing in locations other than the RV apex, which is thought to exacerbate heart failure symptoms). There is a further benefit of moving one or more defibrillation electrodes (either in combination with or independent of one or more pace/sense electrodes) relative to the harness to alter the defibrillation vector, local voltage gradients, and/or impedance to improve the ability to defibrillate the heart. The embodiments disclosed herein relate to various means to provide pace/sense and/or defibrillation electrodes which are coupled to the cardiac harness, yet are movable relative to the harness. Typically the terms “electrode” and “lead” are used to note a specific part of the device as a whole (“electrode” meaning the pace/sense electrode or the defibrillation electrode, and “lead” being the body of the device that contains everything else (conductors, insulation, connectors, etc.)). Sometimes, however, either term is used generically to refer to the lead/electrode device as a whole. This lead/electrode device may have a pace/sense electrode or defibrillation electrode or both.

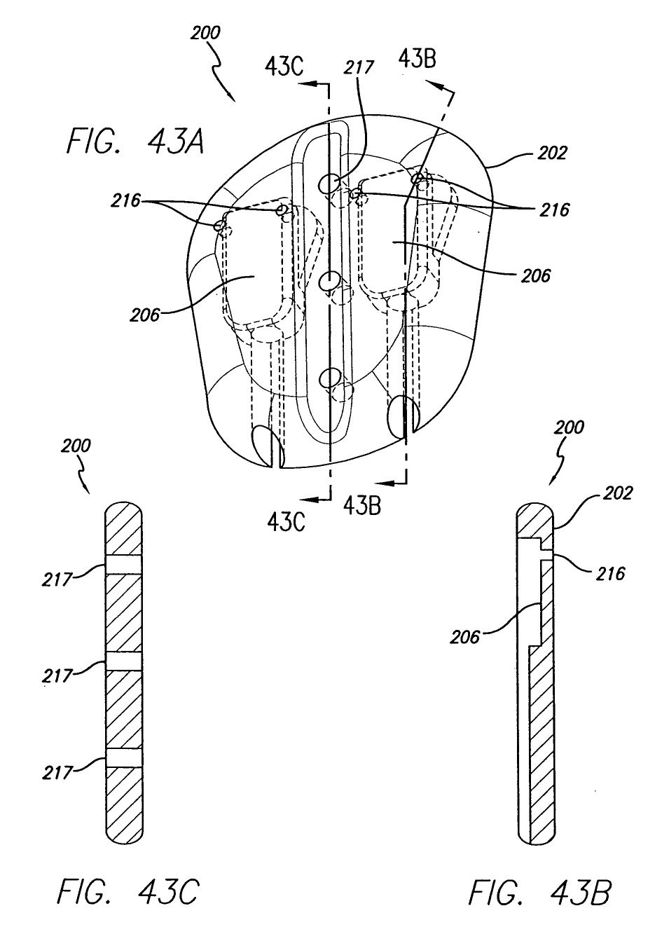

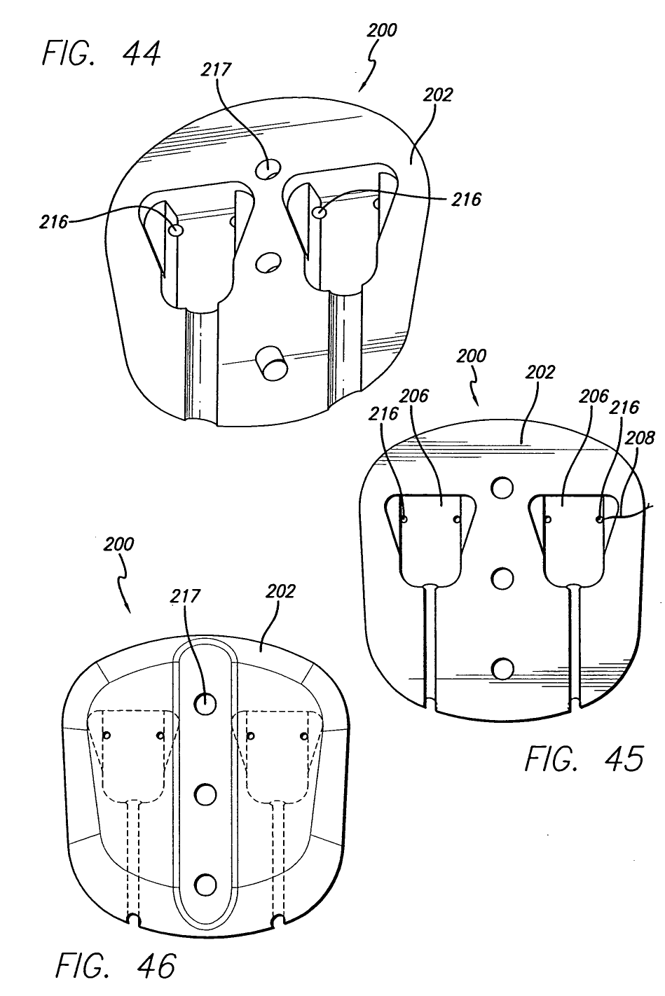

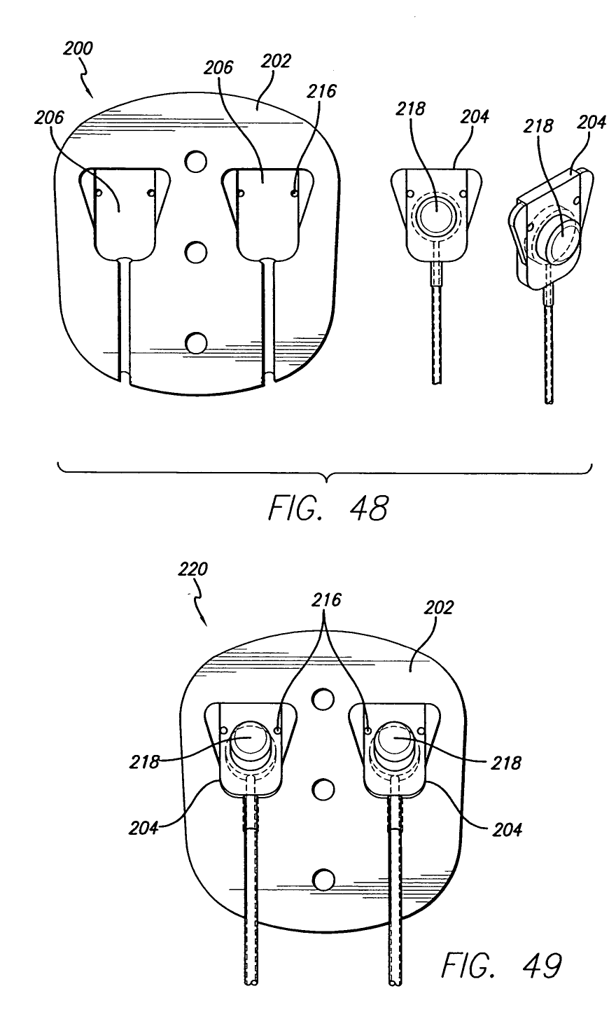

In keeping with the invention, a cardiac harness and assembly is configured to fit at least a portion of a patient's heart and is associated with one or more electrodes capable of providing defibrillation and electrodes used for pacing and/or sensing functions. In one embodiment, shown in FIGS. 43A-49, an adapter 200 having a housing 202 is used to retain one or more pacing/sensing electrodes 204. The adapter is configured to retain the pacing/sensing electrodes so that electrodes are placed in direct contact with the epicardial surface of the heart, or proximate the epicardial surface of the heart. The adapter has a cavity 206 for receiving one or more pacing/sensing electrode and in one embodiment, the cavity is sized and shaped for receiving the pacing/sensing electrodes in an interference fit. In other words, the pacing/sensing electrodes are pressed into the cavity of the adapter in a snap-fit relationship so that there is an interference fit requiring no other fastening means. In another embodiment, a fastener 208 is used to securely retain the pacing/sensing electrodes in the cavity. Fasteners can include, but are not limited to sutures, staples, clips, adhesives, or polymer coatings over the electrodes. Fasteners 208 can be inserted through first apertures 216 and into the adapter 200 in order to more firmly attach the pace/sense electrodes 204 to the cavity 206. In another embodiment, after the pace/sense electrodes are pressed into the cavity, silicone rubber or other dielectric material is molded over the pace/sense electrodes in order to further secure the electrodes in the cavity.

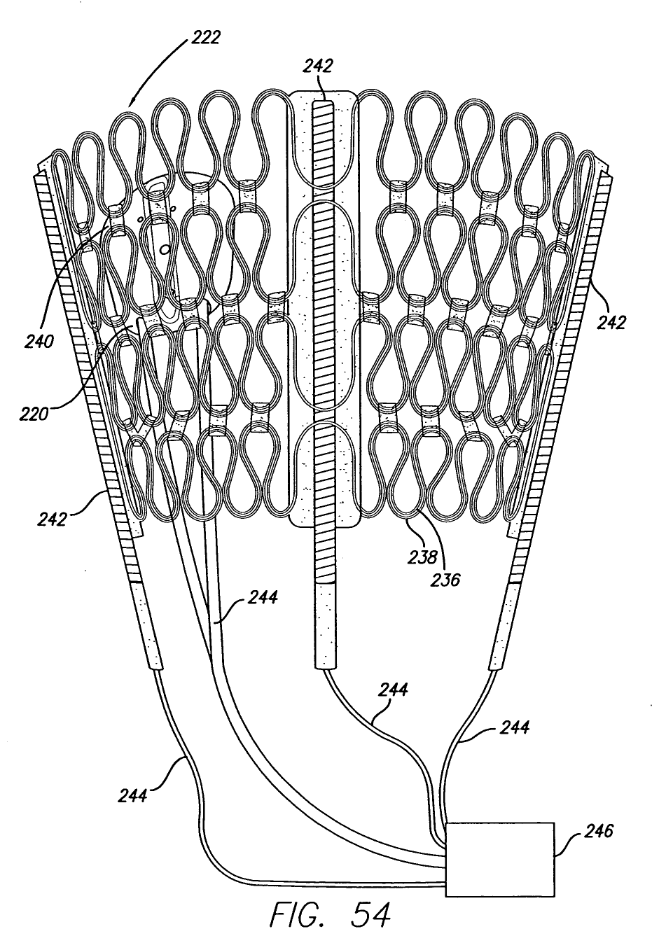

In all embodiments of the adapter 200 disclosed thus far, it is preferred that the cavity 206 be configured to receive the pace/sense electrode 204 so that the electrode 218 on the pace/sense electrode faces away from the cavity. The electrodes 218 typically are in the form of a small metal protrusion or button, such that the button or protrusion extends outwardly from the pace/sense electrode so that the metallic surface of the protrusion or button can come into direct contact with the surface of the heart, or come into nearly direct contact with the surface of the heart. The electrodes 218 are electrically connected to a power source (see FIG. 54). The adapter 200 also has second apertures 217 for receiving release lines as will be further described infra.

In one embodiment, the adapter 200 includes a cavity 206 for receiving a pace/sense electrode 204. After the pace/sense electrode is pressed into the cavity, dielectric material is molded over the pace/sense electrode to retain the pace/sense electrode in the adapter. When molding dielectric material over the pace/sense electrode 204, care must be taken to make sure electrode 218 remains exposed (i.e., not covered). Preferably, the adapter is formed from a silicone rubber material as is the molded layer retaining the pace/sense electrode in the cavity.

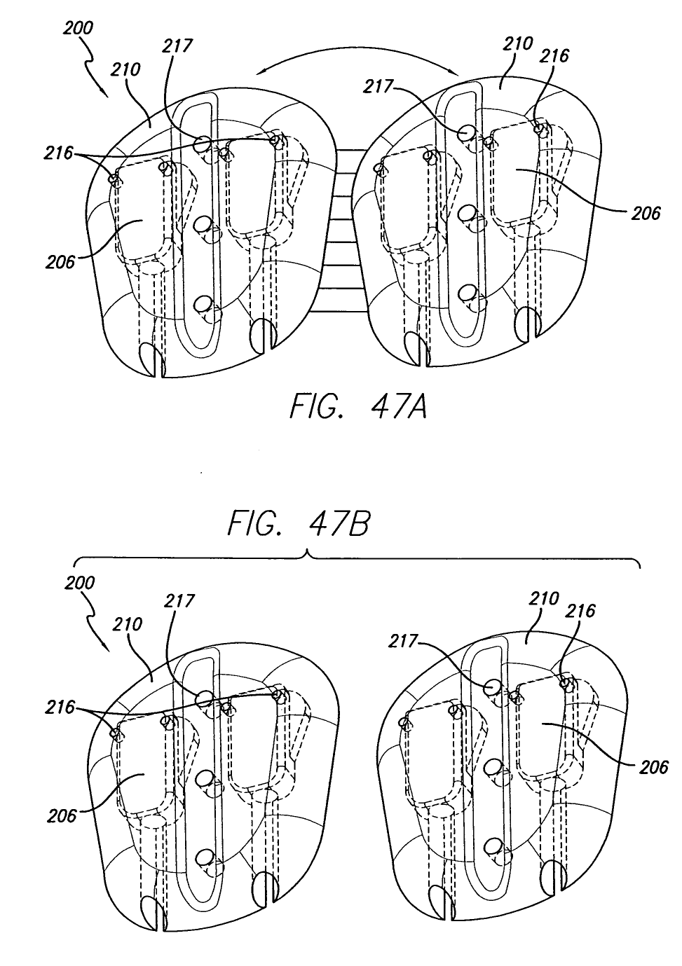

In one embodiment, shown in FIG. 47A, the adapter 200 resembles a clam shell configuration 210 that has an open and closed configuration. In the open configuration (shown in FIG. 47A), the pace/sense electrodes 204 are pressed into cavity 206 and the electrodes are retained in the adapter when the two halves of the clam shell configuration are moved to the closed position (not shown). In another embodiment, shown in FIG. 47B, the adapter 200 is formed in two parts with the cavity 206 formed in a first portion 212 and in a second portion 214. The pace/sense electrodes 204 are pressed into the cavity 206 of either the first portion 212 or second portion 214 and then the first portion is mated to the second portion (not shown) so that the cavity surrounds the pace/sense electrodes. In these embodiments (FIGS. 47A and 47B) an aperture in the cavity corresponds with electrodes 218 so that the electrodes extend through the aperture to directly contact the surface of the heart.

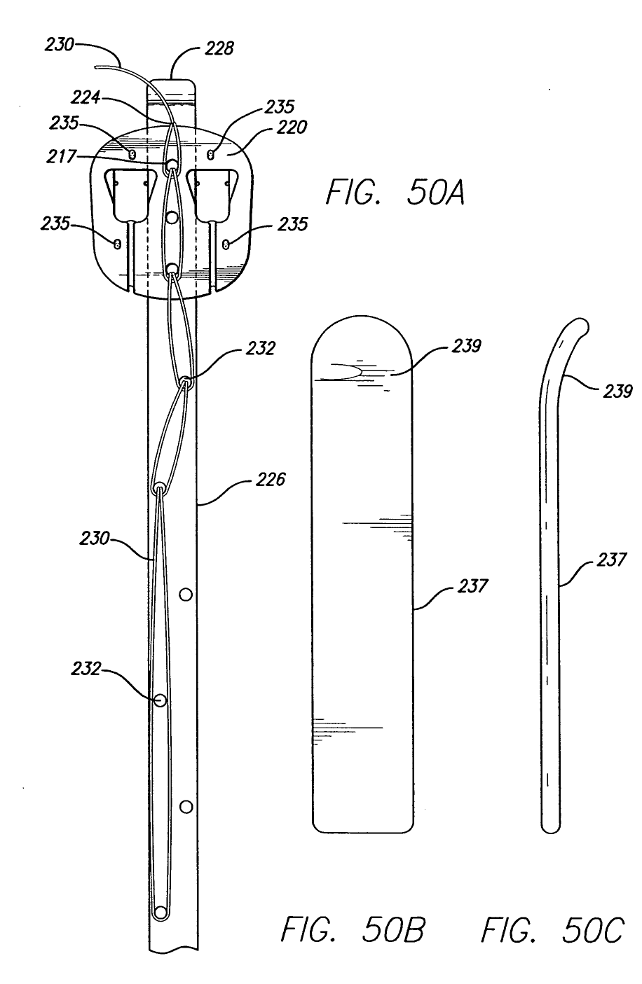

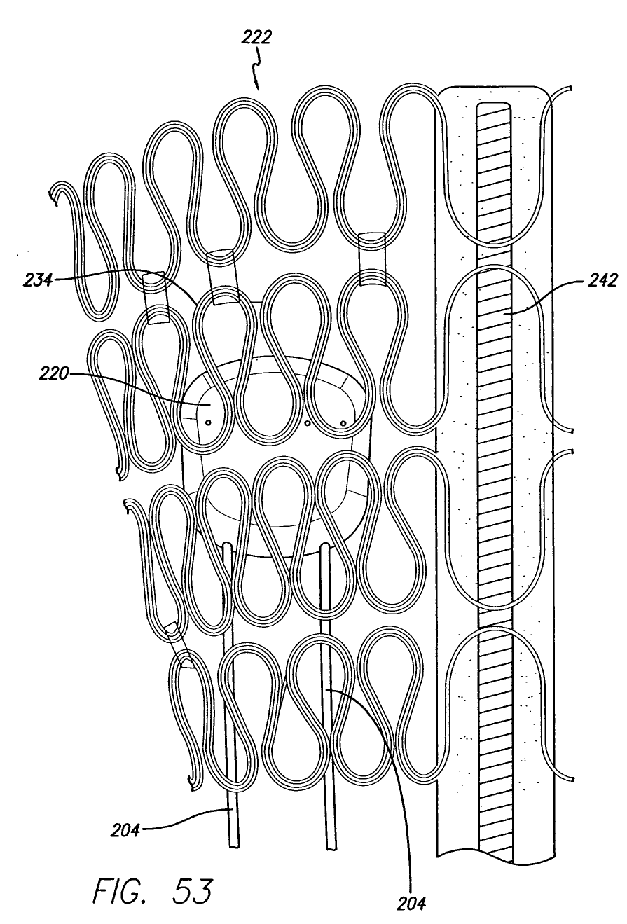

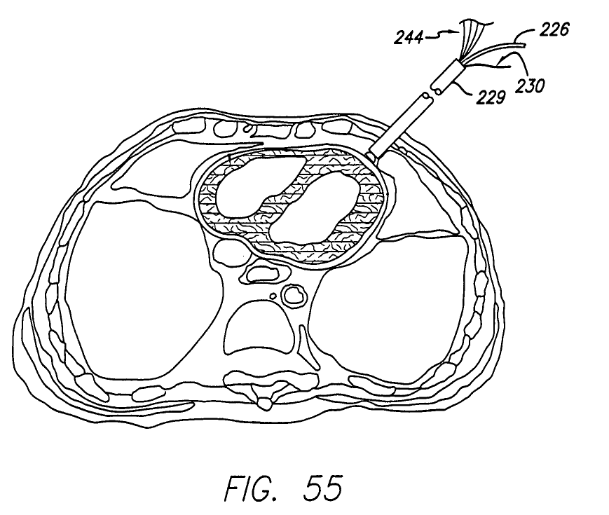

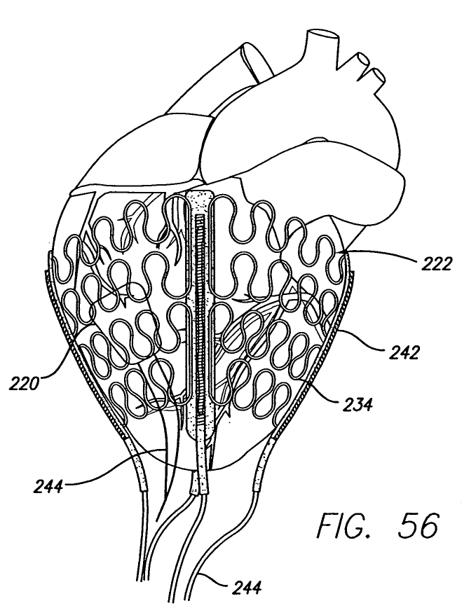

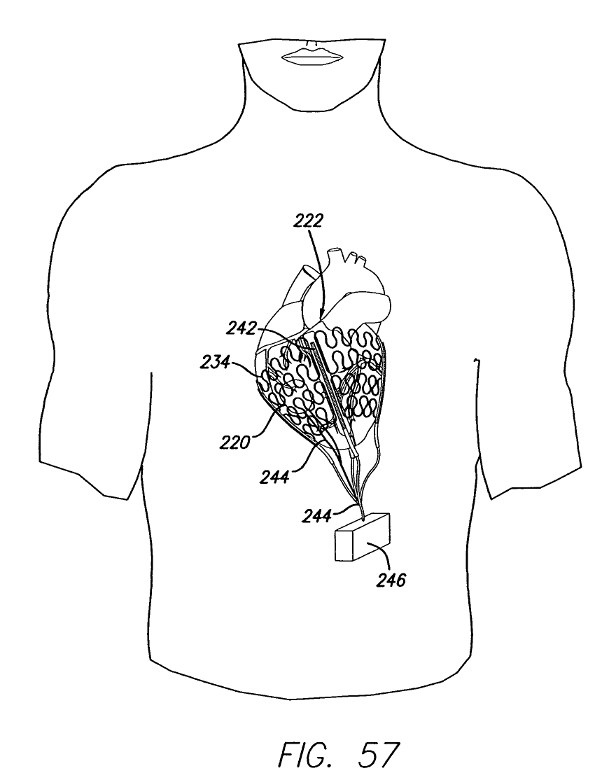

The present invention also includes a method of delivery and a method of use of the adapter and the associated pace/sense electrodes in conjunction with a cardiac harness. Preferably, the mounting of the cardiac harness and placement of the pace/sense electrode under the harness is performed on a beating heart. In one embodiment, shown in FIGS. 50-57, after the pace/sense electrodes 204 have been attached to the adapter 200, an adapter assembly 220 (which includes the adapter with the pace/sense electrodes attached) is positioned under an already implanted cardiac harness 222. Preferably, the adapter assembly is delivered minimally invasively to a desired position under the cardiac harness. In one embodiment, the adapter assembly 220 is releasably attached to the distal end 224 of a push arm 226 which has an atraumatic distal end 228 so that the push arm, with the adapter assembly attached thereto, can be advanced through an introducer tube 229 and under the implanted cardiac harness without catching on or moving the cardiac harness. In this embodiment, the adapter assembly 220 is releasably attached to the push arm 226 by release lines 230. The release lines 230 are wound through third apertures 232 in the push arm 226 and threaded through the second apertures 217 in the adapter in order to releasably attach the adapter assembly to the push arm. The release lines 230 are threaded and tied in a manner similar to that disclosed in U.S. Ser. No. 10/715,150 filed Nov. 17, 2003, the entire contents of which are incorporated herein by reference. After the push arm 226 has been used to position the adapter assembly under the cardiac harness 222, the adapter assembly is released from the push arm by pulling on the release line 230 and the push arm is then withdrawn from the body. As seen for example in FIGS. 52 and 53, the cardiac harness 222 has rows of undulating hinges 234. It is preferred that the adapter assembly 220 be sized to span one or more of the hinges 234 so that the adapter assembly does not protrude through any of the hinges. While the size of the adapter 200 is a matter of choice and can be varied to fit a particular need, the adapter approximate dimensions are about one inch long, one inch wide, and one-eighth inch thick (25.4 mm×25.4 mm×3.2 mm). These dimensions are exemplary, and as stated these dimensions can vary to suit a particular purpose. Since the cardiac harness 222 has a number of rows of undulating hinges 234 that surround the heart and form a slight compressive pressure on the heart, the adapter assembly 220 is held in position on the heart without any further fastening means. Further, if the pericardium is intact, it may provide a slight compressive pressure on the harness and on the adapter assembly as well. Alternatively, a suture or other fastener (not shown) can be used to more securely fasten the adapter assembly 220 to the epicardial surface of the heart. The adapter assembly is positioned under the cardiac harness so that the electrodes 218 on the pacing/sensing electrodes 204 are facing the epicardial surface of the heart and preferably in direct contact with the heart.

While it is believed that the compressive pressure of the cardiac harness 222 on the adapter assembly 220 is sufficient to hold the adapter assembly and pace/sense electrodes 204 firmly onto the epicardial surface of the heart, for added security protrusions 235 can be formed onto the surface of the adapter assembly that faces the cardiac harness 222. The protrusions 235 can, for example, be knobs or raised nubs on the surface of the adapter assembly which will engage the wireform of the cardiac harness, thereby preventing relative movement between the adapter assembly (and pace/sense electrodes) and the cardiac harness. During delivery of the adapter assembly, a sheet of material such as ePTFE or similar material can cover the adapter assembly 220 so that the protrusions 235 do not catch on the cardiac harness 222 as the push arm 226 advances the adapter assembly onto the epicardial surface of the heart. The cover can then be removed after the adapter assembly and pace/sense electrodes are positioned thereby allowing the protrusions 235 to engage with the wireforms of the cardiac harness.

In another embodiment, shown in FIGS. 50B and 50C, a malleable retractor 237 can be used in conjunction with push arm 226 (FIG. 50A) to assist in advancing the push arm and the adapter assembly 220 under the cardiac harness. In this embodiment, the malleable retractor has curved portion 239 that is atraumatic and will not catch on the cardiac harness as the retractor 237 is advanced under the cardiac harness. The malleable retractor is used to create space under the harness for the advancement of the push arm 226 and adapter assembly 220 so that they do not catch on the cardiac harness during delivery. A portion of the retractor 237 can be more flexible than other portions in order to manipulate the retractor under the cardiac harness. The retractor 237 is used to lift portions of the cardiac harness to create free space for the advancement of the push arm and the adapter assembly. Retractor 237 can be used independently or separately from the push arm 226 and adapter assembly 220, or the retractor can be releasably attached to the push arm 226 in order to assist in lifting the harness and creating free space as the push arm and adapter assembly are advanced under the cardiac harness.

It is preferred that the adapter 200 be formed of a dielectric material that is compatible with the material of the cardiac harness 222. In one embodiment, shown in FIG. 54, the cardiac harness 222 is formed of a nitinol alloy wire 236 that is coated with a silicone rubber 238. In this embodiment, the adapter is formed of a silicone rubber 240 as well in order to reduce the frictional engagement between the adapter and the cardiac harness. Further, portions of the pacing/sensing electrodes also can be coated with a dielectric material compatible with the silicone rubber coating on the cardiac harness. Preferably, the pacing/sensing electrodes are also coated with silicone rubber or a similar material in order to reduce frictional engagement and reduce the likelihood of the development of abrasions thereby exposing the bare metal of the cardiac harness or any metal associated with the pacing/sensing electrodes. As will be more fully described, other abrasion resistant materials are contemplated as are materials intentionally designed to abrade that may be useful as coatings on the pace/sense electrodes, adapter, and cardiac harness.

The adapter 200 and the associated pacing/sensing electrodes 204 can be used with any of the embodiments disclosed herein. For example, in one embodiment, shown in FIGS. 54-57, defibrillating electrodes 242 are attached to the cardiac harness 222 for providing a defibrillating shock to the heart. In this embodiment, after the cardiac harness 222 with electrodes 242 is mounted on the heart, the adapter assembly 220 is positioned on the heart under the cardiac harness for the purpose of providing pacing/sensing functions. The leads 244 from the pacing/sensing electrodes 204 and the defibrillating electrodes 242 are connected to a power source 246 (an ICD as previously described). In another embodiment, the cardiac harness, without defibrillating electrodes, is mounted on the heart and the adapter assembly with pacing/sensing electrodes is placed under the cardiac harness for providing pacing/sensing therapy.

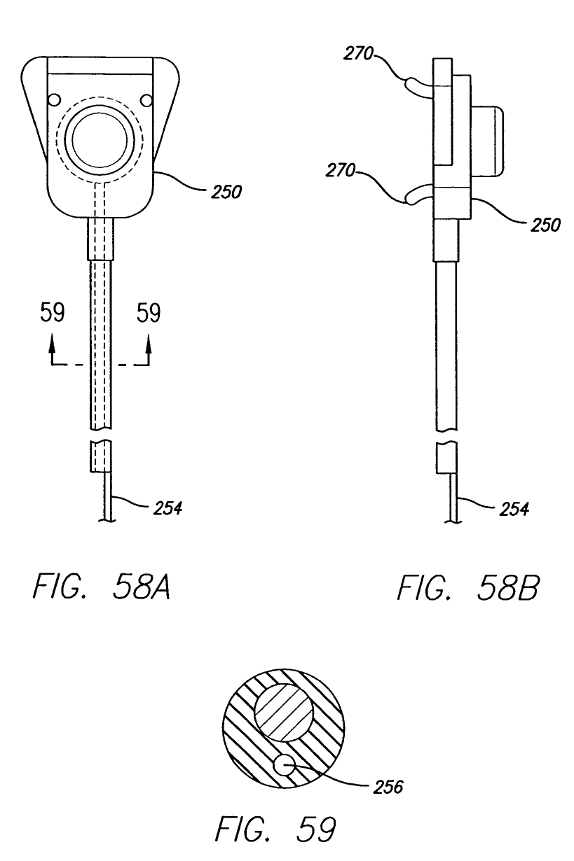

In one embodiment, shown in FIGS. 58A-59, a single pace/sense electrode 250 (with optional defibrillation electrode) is attached to a delivery member that allows it to be slipped under a previously delivered cardiac harness (similar to the embodiment shown in FIGS. 52-53). In this embodiment, the compressive force of the cardiac harness provides the compression required for the pace/sense electrode to firmly contact the heart tissue and to firmly hold the pace/sense electrode onto the epicardial surface of the heart. It may be necessary to provide a surface area on the pace/sense electrode at least as wide as a cell (several hinges) on the cardiac harness to ensure a more even distribution of the compression. A stylet 254 can be inserted and removed from a lumen 256 inside the pace/sense electrode to provide sufficient columnar support during advancement of the pace/sense electrode under the cardiac harness. The stylet 254 is placed in the pace/sense electrode for push force and torquability. The stylet could be straight or shaped round or flat. The stylet provides the ability to advance the pace/sense electrode, move it laterally, or to flip the pace/sense electrode over.

Mechanical features on the pace/sense electrode may help minimize migration of the pace/sense electrode placed under the cardiac harness, and/or minimize relative movement between the materials that could cause material abrasion. As shown in FIG. 58B, one embodiment of a mechanical feature includes protrusions 270 on the pace/sense electrode 250 that are designed to hook within the cardiac harness wireforms and stabilize the pace/sense electrode relative to the harness. The protrusions are rounded, but could have any specific shape that would lend itself to securing each to the wireforms. During delivery, it may be possible to shield or cover the protrusions until the final position is determined. This could be done by covering the protrusions with material and then releasing the material with a release line. A retractable sleeve over the protrusions also could be used. Another embodiment would be to have the protrusions facing the side opposite the harness during delivery, and then torquing the pace/sense electrode to flip the protrusion up against the harness when the final or near-final pace/sense electrode position is attained.

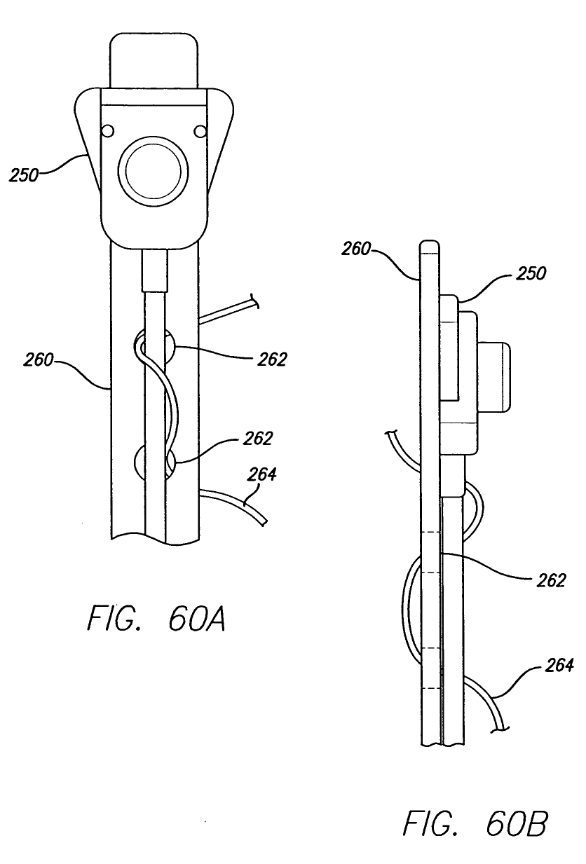

In another embodiment, as shown in FIGS. 60A-60B, a delivery member 260, similar to push arm 226, is used to advance the pace/sense electrode 250 under the cardiac harness. Preferably, the delivery member would be a flattened “paddle-like” member that offers a low profile and resists side-to-side movement during advancement (delivery member 260 is similar to malleable retractor 237). The delivery member may be similar to the current push arm used to deploy the cardiac harness, though it may benefit from being wider and having less of a “nub” at the end, and being either stiffer or more flexible. Delivery member 260 also can be similarly shaped to malleable retractor 237 (FIGS. 50B and 50C) and operate in a similar manner to create space under the cardiac harness as the pace/sense electrode is advanced onto the heart and under the harness. Apertures 262 in the delivery member offer the ability to secure the pace/sense electrode to the member with release lines 264 and release it once it is in the desired position under the cardiac harness. The release lines are tied in the manner previously described and shown in U.S. Ser. No. 10/715,150. As with other embodiments, it is beneficial to connect the proximal end of the pace/sense electrode to a pace/sense analyzer (not shown) prior to releasing the pace/sense electrode from the delivery member to allow the user to make positional adjustments as necessary to optimize the desired electrical performance and/or effect on resynchronization.

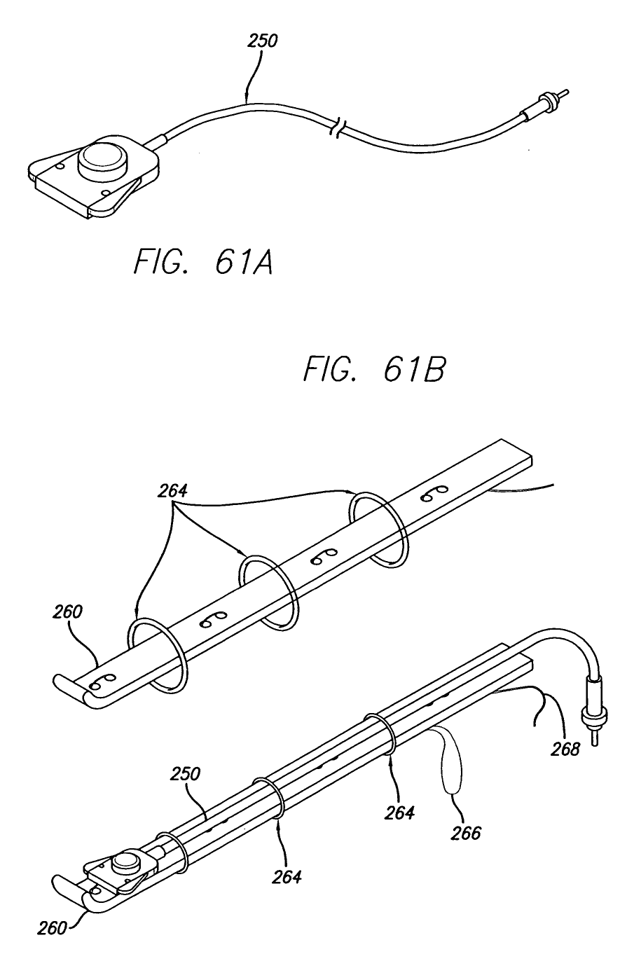

While the pace/sense electrode 250 and delivery member 260 could be manufactured and packaged together, it may be desirable to allow the user the ability to load a separate sterile pace/sense electrode into a sterile delivery member (in the sterile field) at the time of surgery. In one embodiment, as shown in FIGS. 61A-61B, the pace/sense electrode 250 could be inserted under a loose release line mechanism 264 on the delivery member 260 that is then cinched down by the physician prior to delivery. A loop 266 is provided to add tension in order to tighten the release line after the pace/sense electrode is inserted under the loose release line 264. The proximal end 268 of the release line 264 can be pulled to release the loops holding the pace/sense electrode on the delivery member after the pace/sense electrode and the delivery member are advanced under the cardiac harness.

In the embodiments just described, the pace/sense electrode is placed under the harness after the harness has been delivered. There may also be a benefit to having the separate pace/sense electrode be deployed onto the heart at the same time as the cardiac harness. The pace/sense electrodes could be laced to any of the same push arms as the cardiac harness (as seen for example in FIG. 7A), and released onto the heart at the same time as the cardiac harness. The pace/sense. electrode 250 could be laced directly to the cardiac harness 222 shown in FIG. 52 for example (with or without the support of an independent set of push arms). In this case, the release lines 264 attached to the pace/sense electrode 250 and delivery member 252 could be removed independently of the release lines that attach the push arms to the harness. This allows the user to adjust the cardiac harness and pace/sense electrode together after the harness is deployed and the primary delivery system removed.

In another embodiment, the pace/sense electrodes 250 could be laced to delivery members 252 that are positioned under the cardiac harness (before delivery to the heart), but are not attached to the harness. There is an added benefit of this configuration in that the delivery members provide support to the harness to help prevent row flipping and cell interlocks as the harness is advanced onto the heart.

In another embodiment, the delivery members 252 are attached to the same slider as the push arms laced to the cardiac harness (similar to FIGS. 27-35) and all release lines are connected to the same pull ring. In another embodiment (not shown), the delivery members are attached to a separate sliding mechanism, preferably in front of the slider to which the push arms carrying the cardiac harness are connected. Alternatively, there could be one sliding mechanism, but the delivery members could be detached from it after deployment onto the heart. At this point, usage of the delivery members would be similar to the case of having a separate sliding mechanism. Either way, the release lines from the pace/sense electrodes and the cardiac harness are connected to separate removal mechanisms. The pace/sense electrodes may be able to be released independently of the defibrillating electrodes. The delivery members may also be removed from the slider independently of one another. This allows the pace/sense electrodes to be advanced either ahead of or with the cardiac harness. It also allows the removal of the primary cardiac harness delivery system, leaving behind the delivery members attached to the pace/sense electrodes. Each pace/sense electrode may then be manipulated under the harness as necessary before being released from the delivery member.

It should be noted that the same or similar pace/sense electrode delivery techniques described above could be used to deploy a pace/sense electrode 250 onto any position on the surface of the heart, including the right or left atrium. There are particular advantages of being able to place a pace/sense electrode on the left atrial epicardial surface. As is typically recommended for CRT procedures, atrial sensing and optional pacing allows for improved timing between atrial and ventricular contractions (assuming a ventricular pace/sense electrode is present). Placement of a pace/sense electrode onto the atrial epicardial surface prevents the need for venous access to the right atrium, thus allowing the cardiothoracic surgeon to perform the whole procedure. It also allows the possibility of left atrial electrode placement, which is not feasible from a venous approach. Left atrial sensing and optional pacing particularly optimizes left atrial and left ventricular contraction timing.

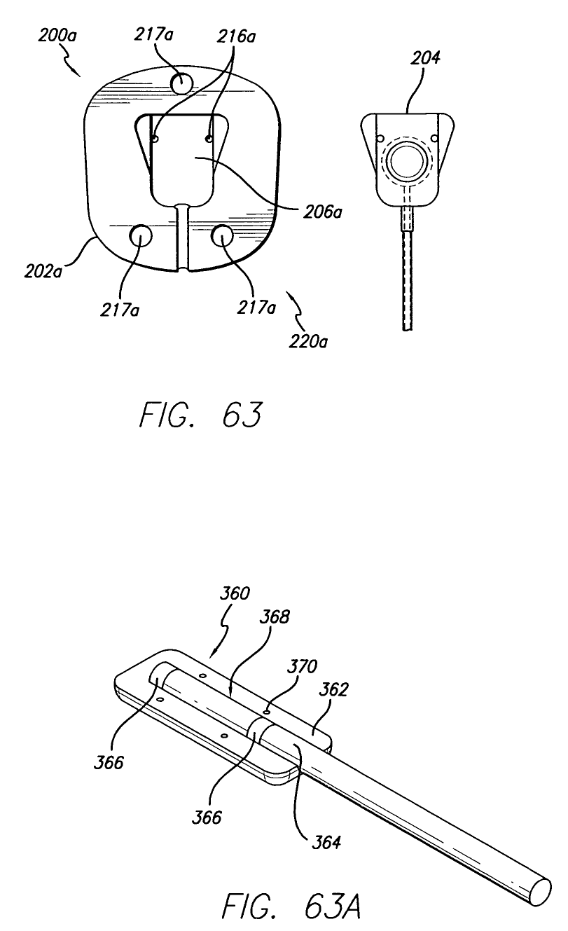

In another embodiment, shown in FIG. 63, a single adapter 200a having a housing 202a is used to retain one pacing/sensing electrode 204. The adapter is configured to retain the pacing/sensing electrode so that the electrode is placed in direct contact with the epicardial surface of the heart, or proximate the epicardial surface of the heart. The adapter has a cavity 206a for receiving one pacing/sensing electrode and in one embodiment, the cavity is sized and shaped for receiving the pacing/sensing electrodes in an interference fit. In other words, the pacing/sensing electrode is pressed into the cavity of the adapter in a snap-fit relationship so that there is an interference fit requiring no other fastening means. The single adapter 200a may have the same characteristics as the adapter 200 shown in FIG. 43a, including the same material and it may also have a similar size so the cardiac harness has enough surface area to contact and hold the single adapter 200a without slipping through the wireforms of the cardiac harness.

As discussed above in relation to the adapter 200, a fastener may also be used to securely retain the pacing/sensing electrode 204 in the cavity 206a of the single adapter 200a. Fasteners can include, but are not limited to sutures, staples, clips, adhesives, or polymer coatings over the electrodes. Fasteners can be inserted through first apertures 216a and into the adapter 200a in order to more firmly attach the pace/sense electrodes 204 to the cavity 206. In another embodiment, after the pace/sense electrodes are pressed into the cavity, silicone rubber or other dielectric material is molded over the pace/sense electrode in order to further secure the electrodes in the cavity.

In all embodiments of the single adapter 200a disclosed thus far, it is preferred that the cavity 206a be configured to receive the pace/sense electrode 204 so that the electrode 218 on the pace/sense electrode faces away from the cavity. In use, existing pace/sense electrodes may be fitted into the cavity 206a of the single adapter 200a to form a single adapter assembly 220a. Existing pace/sense electrodes may also be used with the adapter 200 discussed above. An example of an existing pace/sense electrode is a Capsure Epi Lead manufactured by Medtronic Inc., Minneapolis, Minn. The single adapter 200a also has second apertures 217a for receiving release lines as described above in with regard to the adapter 200.

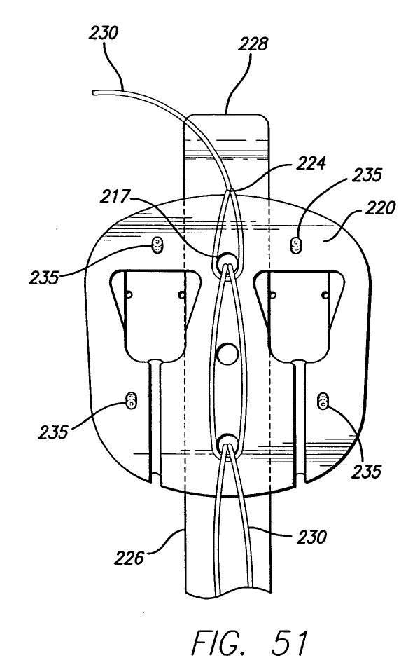

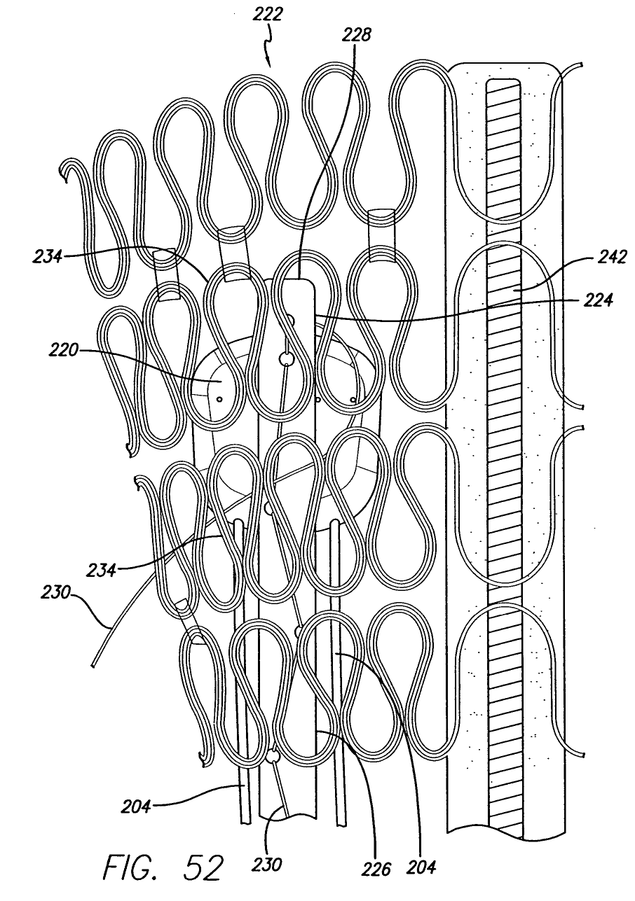

While it is believed that the compressive force of the cardiac harness 222 on the single adapter assembly 220a is sufficient to hold the single adapter assembly and pace/sense electrode 204 firmly onto the epicardial surface of the heart, for added security, protrusions (not shown) can be formed onto the surface of the adapter assembly that face the cardiac harness 222. These protrusions are similar to the protrusions 235 on the adapter 200 shown in FIG. 51. As described above, during delivery of the single adapter assembly 220a, a sheet of material such as ePTFE or similar material can cover the single adapter assembly so that the protrusions do not catch on the cardiac harness 222 as a push arm advances the adapter assembly onto the epicardial surface of the heart. The cover can then be removed after the single adapter assembly or multiple single adapter assemblies are positioned thereby allowing the protrusions to engage with the wireforms of the cardiac harness.

In use, one or more of the single adapter assemblies 220a can be delivered to the heart and positioned under the cardiac harness 222 using the same methods as described above in regard to the adapter 200 and adapter assembly 220. In another embodiment, the single adapter assemblies 220a can be delivered to the heart using the same methods as described above in regard to the pace/sense electrodes 250. Placing two single adapter assemblies 220a under the cardiac harness has the same advantages as placing two pace/sense electrodes 250 as described above.

In another embodiment, shown in FIG. 63A, a sinus lead adapter 360 having a housing 362 is used to retain a coronary sinus lead 364. The adapter is configured to retain the coronary sinus lead so that electrodes 366 of the lead are placed in direct contact with the epicardial surface of the heart, or proximate the epicardial surface of the heart. The electrodes on the coronary sinus lead are ring electrodes and the lead may include 1, 2, 3, or more electrodes at its distal tip. The adapter has a cavity 368 for the coronary sinus lead, and in one embodiment the cavity is sized and shaped for receiving the pacing/sensing electrodes in an interference fit. In other words, the lead is pressed into the cavity of the adapter in a snap-fit relationship so that there is an interference fit requiring no other fastening means. The sinus lead adapter may be formed of a dielectric material, such as silicone rubber. In use, the adapter may favorably insulate the portion of the electrode ring not in contact with the surface of the heart. The insulation provided by the adapter limits current loss and prevents phrenic nerve stimulation. Further, due to the positioning of the lead in the adapter, any steroid emitted from a steroid collar may possibly be more concentrated on the epicardial surface. The size of the sinus lead adapter is such that the compressive forces of the cardiac harness hold the adapter on the surface of the heart without the adapter slipping through the wireforms of the cardiac harness.

A fastener may also be used to securely retain the coronary sinus lead 364 in the cavity 368 of the sinus lead adapter 360. Fasteners can include, but are not limited to sutures, staples, clips, adhesives, or polymer coatings over the electrodes. Fasteners can be inserted through apertures 370 and into the adapter in order to more firmly attach the coronary sinus lead to the cavity. In another embodiment, after the coronary sinus lead is pressed into the cavity, silicone rubber or other dielectric material is molded over the lead in order to further secure the electrodes in the cavity. In this embodiment, care must be taken not to cover the surfaces of the ring electrodes 366 with the dielectric material.

Existing coronary sinus leads may be used with the sinus lead adapter 360. An example of an existing coronary sinus lead is the Quicksite manufactured by St. Jude Medical, Inc. The coronary sinus lead shown in FIG. 63A has bipolar ring electrodes, and it has been contemplated that a unipolar coronary sinus lead may also be fitted in the adapter. The adapter may also include apertures (not shown) for receiving release lines as described above in with regard to the adapter 200.

In use, one or more of the sinus lead adapters 360 retaining coronary sinus leads 364 can be delivered to the heart and positioned under the cardiac harness using the same methods as described above in regard to the adapter 200 and adapter assembly 220. It has also been contemplated that coronary sinus leads may be delivered and positioned between a cardiac harness and the surface of the heart without the use of the adapter 360. In one embodiment, the coronary sinus lead, with or without the adapter, may be delivered to the heart and positioned under a cardiac harness by itself without the aid of delivery member, such as a push arm.

While the compressive force of the cardiac harness is sufficient to hold the sinus lead adapter 360 firmly onto the epicardial surface of the heart, for added security, protrusions (not shown) can be formed onto the back surface of the adapter that face the cardiac harness. These protrusions are similar to the protrusions 235 on the adapter 200 shown in FIG. 51. As described above, during delivery of the adapter, a sheet of material such as ePTFE or similar material can cover the adapter so that the protrusions do not catch on the cardiac harness as a push arm advances the adapter onto the epicardial surface of the heart. The cover can then be removed after one or more sinus lead adapters are positioned thereby allowing the protrusions to engage with the wireforms of the cardiac harness.

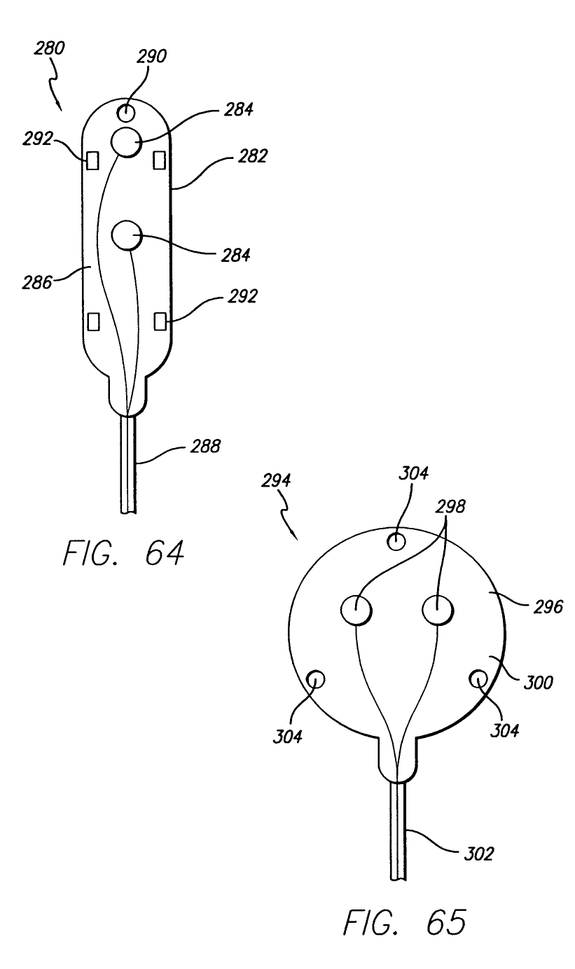

In another embodiment, shown in FIG. 64, a moveable or modular pace/sense electrode spine 280 includes a spine body 282 having a “paddle-like” shape that retains one bipolar pair of button type electrodes 284 exposed on a front surface 286 of the spine body. The “paddle-like” shape of the modular electrode spine has a low profile. It has also been contemplated that this could be a unipolar electrode with a single electrode disposed on the spine body. The electrodes can be configured with a steroid eluting component to reduce scar tissue development and prevent exit block from forming. In this embodiment, the modular pace/sense electrode spine is configured with a standard IS-1 type connector. The electrodes 284 are in communication with a power source, and in the embodiment shown, the associate leads 288 are connected to a power source. There is also an aperture 290 disposed through the modular spine for receiving release lines in the same manner as described above with regard to the adapter 200.

The spine body 282 can be formed with a dielectric material that is molded over the pair of pace/sense electrodes 284, and care must be taken to make sure the surface of the electrodes remain exposed so that they can be positioned on the epicardial surface of the heart. It is preferred that the spine body is formed of a silicone rubber material. In order to reduce frictional engagement between the modular spine 280 and the cardiac harness and reduce the likelihood of development of abrasions, the modular spine can be backed with ePTFE or can be plasma treated as discussed in detail below. The modular spine may also include grip pads 292 attached to the front surface 286 of the spine body 282 to add a self-anchoring feature to the modular spine. The spine body provides a large surface area that comes in contact with the cardiac harness to remain positioned on the heart. It is contemplated that the spine body in this embodiment may have a length between about 3 cm and about 10 cm and a width between about 0.5 cm and about 4 cm.

FIG. 65 shows another embodiment of a moveable or modular pace/sense electrode spine 294. The embodiment includes a spine body 296 with a low profile having a general shape of a circle that retains one bipolar pair of button electrodes 298 exposed on a front surface 300 of the spine body. In this embodiment, the pair of electrodes are placed side-by-side horizontally, however, they may also be placed linearly in a column, or diagonally. It has been contemplated that a single electrode be disposed on the spine body and that the spine body may be any geometric shape such as square, rectangle, or star. The electrodes are in communication with a power source, and in the embodiment shown, the associated leads 302 connect to a power source (not shown).

There also are release apertures 304 disposed through the spine body 296 for receiving release lines in the same manner as described above with regard to the adapter 200 for delivery of the modular spine. The spine body can be formed with a dielectric material that is molded over the pair of pace/sense electrodes 298. It is preferred that the spine body is formed of a silicone rubber material. In order to reduce frictional engagement between the modular spine 294 and the cardiac harness and reduce the likelihood of development of abrasions, the modular spine can be backed with ePTFE or it can be plasma treated. The modular spine may also include grip pads (not shown) attached to the front surface of the spine body to help prevent sliding or other movement once the modular spine is positioned on the heart and under the cardiac harness. The spine body provides a large surface area to come in contact with the cardiac harness and remain positioned on the heart. It is contemplated that the spine body in this embodiment have a diameter between about 2 cm and about 8 cm.

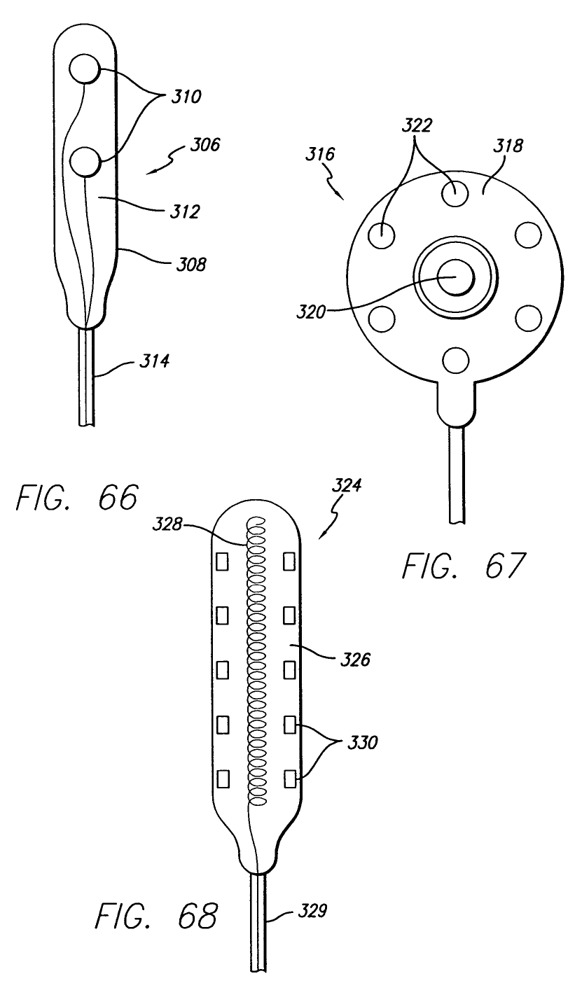

Another embodiment is shown in FIG. 66 of a moveable or modular pace/sense electrode spine 306 that includes a low profile spine body 308 shaped as a stylet that retains one pair of button electrodes 310 exposed on a front surface 312 of the spine body. It has also been contemplated that that a single electrode be disposed on the spine body. The electrodes are in communication with a power source, and in the embodiment shown, the associate leads 314 are connected to a power source. An aperture may be disposed through the spine body for receiving release lines in the same manner as described above with regard to the adapter 200. When delivering this embodiment of the modular spine, the release line associated with a push arm or stylet can be tied around spine body 308.

The spine body 308 can be formed with a dielectric material that is molded over the pair of pace/sense electrodes 310, and care must be taken to make sure the surface of the electrodes remain exposed so that they can be positioned on the epicardial surface of the heart. It is preferred that the spine body is formed of a silicone rubber material. In order to reduce frictional engagement between the modular spine and the cardiac harness and reduce the likelihood of development of abrasions, the modular spine may be backed with ePTFE or may be plasma treated. The modular spine may also include grip pads or protrusions (not shown) attached to the spine body to help prevent sliding or other movement once the modular spine 306 is positioned on the heart and under the cardiac harness. It is contemplated that the spine body in this embodiment have a width of about 0.5 cm and the length may vary between about 2 cm and about 10 cm.

FIG. 67 shows another embodiment of a moveable or modular pace/sense electrode spine 316 with a different electrode configuration. This embodiment includes a low profile, circular shaped spine body 318 having an Omni directional bipolar electrode pair 320. Another difference with this embodiment is that it includes disc shaped grip pads 322, which could be any geometry and placed in any configuration in order to hold and increase the functional engagement between the electrodes and the epicardial surface of the heart. The spine body can be formed with a dielectric material that is molded over the Omni directional bipolar electrode pair. It is preferred that the spine body is formed of a silicone rubber material.

In keeping with the invention, a moveable or modular defibrillation electrode may also be mounted under the cardiac harness that is placed on a beating heart. FIG. 68 shows a defibrillation spine 324 with a spine body 326 retaining a defibrillation electrode coil 328. The defibrillation electrode is in communication with a power source, and in the embodiment shown, the associate lead 329 is connected to a power source. Although not shown, there may also be apertures disposed through the spine body for receiving release lines associated with a push arm in the same manner as described above with regard to the adapter 200. The spine body can be formed with a dielectric material that is molded over the defibrillation electrode, and care must be taken to make sure a portion of the coil remains exposed so it can be positioned on the epicardial surface of the heart. It is preferred that the spine body is formed of a silicone rubber material. In order to reduce frictional engagement between the defibrillation lead and the cardiac harness and reduce the likelihood of development of abrasions, the defibrillation lead can be backed with ePTFE or can be plasma treated. The defibrillation lead may also include grip pads 330 attached to the spine body to help self-anchor the defibrillation lead. The spine body provides a large surface area to come in contact with the cardiac harness and remain positioned on the surface of the heart.

Using the defibrillation spine 324 may be useful in adding another electrode for defibrillation where an additional current vector would be useful to lower the defibrillation threshold. The defibrillation spine can be used in place of sub muscularly placed patch electrodes and may be more effective since it is placed on the epicardial surface for minimal energy loss. In addition, epicardial placement would allow for easier manipulation to get the exact vector needed to optimize therapy. It has been contemplated that the defibrillation spine shown in FIG. 68 could also be a patch, array, or other type of defibrillation electrode configuration instead of a defibrillation electrode coil.

In the embodiments described herein, consideration is made for the interaction of the cardiac harness and the pace/sense electrode, which relies on the tension of the harness to hold the electrode in place. It may be that once the harness and pace/sense electrode are fibrosed in place, little relative motion exists. However, this may not be the case requiring features in the pace/sense electrode and/or the cardiac harness to minimize relative movement between the devices, or if relative motion exists, minimize the friction or propensity for material abrasion in the chronic setting. Because silicone rubber in its unaltered cured state can abrade against itself and against other materials, it may be important to utilize implantable materials in the cardiac harness 222, adapter 200, the pace/sense electrodes 204, 250, 280, 294, 306, 316, and/or the defibrillation spine 328 that are positioned against it, that have more abrasion resistant surfaces. Examples of abrasion resistant materials include, but are not limited to: application of a lubricious silicone oil or hydrophilic coating to the pace/sense electrode body surface; silicone extruded tubing (e.g., platinum-cured Nusil 4755) which has the surface modified with plasma; oxidative reduction of the silicone surface to a silicon suboxide; plasma enhanced chemical vapor deposition of a silicon suboxide (these processes should reduce the tackiness of the surface and increase toughness); silicone extruded tubing that has a TEFLON or Parylene deposited upon the surface; a sleeve of TEFLON or ePTFE over the surface of the adapter 200 or the pace/sense electrode 204, 250, 280, 294, 306, 316 or the defibrillation spine 328 (the material could also be used in place of silicone); a matrix of braided or wound fibers (e.g., TEFLON, polypropylene, or polyester) or a matrix of an otherwise porous material (e.g., ePTFE), impregnated with silicone or another implantable elastic material; silicone extruded tubing with a layer of polyurethane (e.g., 55D polyurethane, a more lubricious and abrasion resist implantable material) over the surface (either as a sleeve slipped over the surface, a sleeve melted down onto the surface, or coextruded onto the surface); polyurethane used in place of silicone; and a chemical blend of silicone and polyurethane, such as Elast-Eon 2A, produced by Aortech Biomaterials plc. In one embodiment, the cardiac harness 222 has a coating of silicone rubber over the nitinol wireform. The adapter 200, the pace/sense electrodes 204, 250, 280, 294, 306, 316, and/or the defibrillation spine 328 are constructed with a sheet of ePTFE over the devices which will not only reduce contact force (and frictional force) but the wireform of the cardiac harness will sink down to be flush with the top surface of the ePTFE thereby reducing the contact force (and frictional force) to zero. Once the contact and frictional forces are substantially reduced, the frictional and wear abrasion between the two devices are effectively eliminated.

Another embodiment of the pace/sense electrode 250 is that it has a geometry in the region of the electrodes that is wider than the rest of the lead, preferably at least as wide as one or more hinges on the cardiac harness wireform, to help distribute the contact force of the harness against the pace/sense electrode. A reduction in contact force should help reduce the propensity of the material to abrade. Also, the material on the harness wireform side of the electrode is preferably an abrasion resistant material, similar to those described above, but in this case preferably constructed from an ePTFE sheet. Besides being flexible and lubricious implantable material, the ePTFE has the advantage of allowing silicone, molded around the lead components, to impregnate its matrix and form a secure bond. An alternative to the ePTFE sheet would be a “fabric” or “mesh” of fibers, such as polyester.

There also is a benefit to a method of using a malleable retractor (or similar blunt, flat tool) to lift an already deployed harness (by placing the tool under the harness and lifting it away from the heart or turning on its edge) and inserting the pace/sense electrode or defibrillation lead under the tool. Such a tool could be a malleable retractor, or other customized flat, stiff, low-profile tool to create the desired space. The tool serves to provide a clear path for inserting the lead without hang-ups on the harness. Once the pace/sense electrode is under the harness the tool may be removed.

While the focus is on pacing, sensing, and defibrillation electrodes, the concepts also may be applied to any other sort of sensor placed on the heart (e.g., magnetic, ultrasound, pH, impedance, etc.).

One advantage of a pace/sense electrode not attached to the cardiac harness, is that it allows the physician to scout a position for the pace/sense electrode. This could be done before deploying the harness, after deploying the harness but before deploying the pace/sense electrode, or after deployment of both the harness and the implantable pace/sense electrode with the intent to move the implantable electrode to provide a better target. A combination of the above techniques could also be accomplished. For example, the scout electrode could be used first to target a position, and then used again after deployment of the implantable pace/sense electrode to help confirm or adjust the proper position of the pace/sense electrode. Scouting involves moving an electrode around the surface of the heart to find a target location to position the implantable pace/sense electrode. This location is determined by a combination of the desired anatomic location of the electrode, the quality of the electrogram, and the ability to pace the site. Importantly, one could use the same pace/sense electrode for scouting as that intended for permanent implantation. If such an electrode is used for scouting and it contains a steroid eluting plug or collar, it may be important to provide a resorbable coating over the electrode to prevent early loss of the steroid before it is in the final implant position. Such a coating could be mannitol or polyethylene glycol (PEG). In another embodiment, one could use a non-implantable electrode probe to scout the desired position. By not being permanently implanted, this probe may more easily incorporate the following features: cheaper to make and use; potentially reusable; easier to use; it could be made with a specific feature to improve tissue contact (pre-shape curve, use of a steerable handle, or other stiffening/maneuvering mechanism); have multi-electrode capability with a multi-pin connector to allow the ability to easily switch between electrodes at the proximal end (this also would allow the ability to connect to a multi-electrode mapping system, e.g., Bard EP, Pruka, Biosense, etc. for quick assessment of the ideal location); and anatomic positioning could be enhanced with the incorporation of sensors to identify the position of the electrodes relative to the heart and relative to adequately conductive tissue. Examples of such sensors include magnetic hall sensors (such as used in the J&J/Biosense catheters), or ultrasound sensors (such as used in the Boston Scientific/Cardiac Pathways catheters).

With some of the embodiments disclosed herein, the order of the deployment of the cardiac harness and the pace/sense electrodes or defibrillation lead may vary: deploy the pace/sense electrode and/or defibrillation lead then the harness; deploy the harness and the pace/sense electrode and/or defibrillation lead at the same time; and/or deploy the harness then deploy the pace/sense electrode and/or defibrillation lead.

In the disclosed embodiments, it is preferred that the implantable pace/sense electrode 250 be deployed under the pericardium from an opening at the apex. However, it is possible that the electrode could be deployed from outside the pericardium. To accomplish this, a slit in the pericardium, somewhere other than at the apex would be made, and the pace/sense electrode advanced onto the epicardium through the slit. The potential advantage of this approach would be to allow the pericardium to act as a means to prevent direct contact (that could cause material wear) between the pace/sense electrode body and cardiac harness. The slit could be a small incision in the range of about 0.25 inch to about 1.00 inch (1.016 mm to 25.4 mm) and the incision could be closed with a suture (or other fastener like a staple) around the lead.

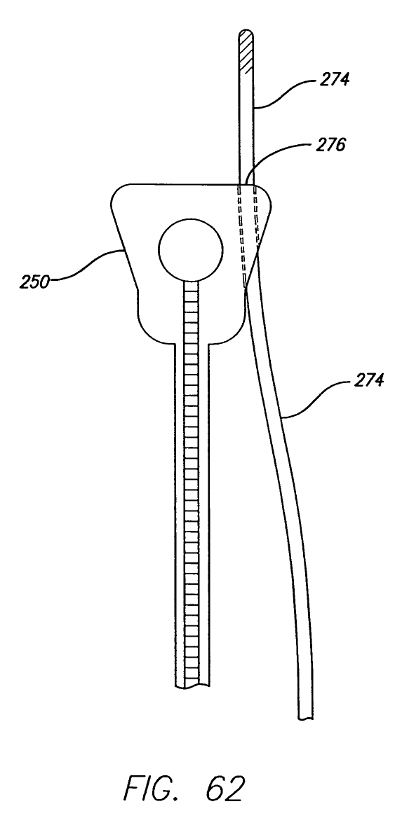

The emphasis for the delivery mechanism listed below are on the implantable pace/sense electrode, but could apply to a non-implantable scouting probe as well. In one aspect of the invention, shown in FIG. 62, the pace/sense electrode 250 is advanced over a guidewire 274, that is atraumatic and has precise steering. The guidewire extends through a lumen 276 in the pace/sense electrode so that after the guidewire is positioned under the pericardium, the pace/sense electrode is advanced over the guidewire and into contact with the epicardial surface of the heart. After the pace/sense electrode is in position, the guidewire is withdrawn from the patient. Lumen 276 can be positioned anywhere on or through the pace/sense electrode 250. For example, the lumen could extend through the lead wire or coaxially next to the lead wire and through the pace/sense electrode so that the lumen extends all the way through the entire pace/sense electrode and associated leads. The guidewire preferably is inserted through a small incision in the pericardium as previously described. The guidewire could be advanced atraumatically beyond the AV groove.

Secure contact between the pace/sense electrode and myocardium is important for optimal sensing and pacing. The following features allow the ability to fix the pace/sense electrodes securely to the epicardial surface of the heart. One can use the pericardium to hold the pace/sense electrode against the epicardial surface. One can use the cardiac harness to compress the pace/sense electrode and/or pace/sense electrode body against the heart. An expandable member (such as an expandable balloon, not shown) is positioned on the pericardial side of pace/sense electrode (pace/sense electrode placed in space between epicardium and pericardium). If the pace/sense electrode is on the outside of the harness, the expandable member expands against pericardium and forces electrodes into the epicardium. If the pace/sense electrode is under the harness, the expandable member expands against the harness and also the pericardium to force the electrodes into contact with the epicardium. Examples of an expandable member include an inflatable bladder (using air or fluid), or an expandable cage (e.g., nitinol wireforms). The member could be self-expanding or expanded by the user. Other features used to fix the pace/sense electrode to the epicardial surface of the heart include: tissue adhesive (a lumen in the pace/sense electrode with a distal port at one or more locations on the pace/sense electrode, including positions near the electrode, could be used to transport a tissue adhesive, e.g., cyanoacrylate, that would fix lead to the epicardial and/or pericardial tissue); a pre-filled bladder of adhesive could also be punctured to allow the adhesive to dispense; an elastic band (elasticity achieved through strain of a metal wireform such as the nitinol in the harness or with an elastic rubber-like polymer wherein the band would be attached to the electrode and then made to elongate around the heart or relative to points/devices fixed relative to the heart); or friction pads (the friction of features on the pace/sense electrode help hold the pace/sense electrode and/or electrodes against the heart surface).

In another aspect, the material at the cardiac harness-pace/sense electrode interface could be made of a soft material that helps the harness settle into the lead material. This could be a porous or foam-like material, or a matrix of thin protrusions on the surface, to create a brush-like or carpet-like surface, into which the harness settles. There also may be an advantage to having the outer layer of the pace/sense electrode in contact with the cardiac harness and/or the material on the harness itself, consist of a soft material that compresses or dimples when the harness wireforms are pressed against it. This may help reduce the contact pressure between the pace/sense electrode and the harness, as well as to help the materials lock into one another, especially when fibrosed in place. In another aspect, the material at the cardiac harness-pace/sense electrode interface could be made of a tacky material, such as a gel or low-durometer silicone, that helps the materials to stick to one another. In another aspect, the material on the pace/sense electrode and/or the cardiac harness could be designed to ensure that the tissue grows in and around the pace/sense electrode and harness, linking them together. Examples of such materials include ePTFE, DACRON, and porous silicone. Pore size could be 10-100 microns, preferably 20-30 micron. If a porous material (e.g., fiber mesh, ePTFE, or other open cell polymer matrix) is used on the pace/sense electrode, the final open pore size may be optimized to achieve certain features of the pace/sense electrode, depending on where and how the pace/sense electrode is used. It may be desirable to limit the pore size to minimize tissue in-growth and facilitate later removal of the pace/sense electrode, or a portion of the pace/sense electrode, if it ever became necessary. However, in the region adjacent the cardiac harness wireforms, there may be an advantage of encouraging tissue in-growth that could serve to stabilize the pace/sense electrode and/or cardiac harness and minimize relative movement between the two. The above mentioned brush-like or carpet-like features could also enhance tissue in-growth. The material could also be selectively coated or impregnated with a drug that promotes fibrin deposition for an enhanced acute effect.

In one aspect of the invention, there are various materials that can be chosen for use on both the pace/sense electrode and cardiac harness to resist abrasion between the two. In addition, composite designs may also resist abrasion. Coils, braids, and/or weaves of metal (e.g., stainless steel, nitinol, platinum, MP35N), or abrasion-resistant polymers (e.g., polyester, polyimide, TEFLON, KEVLAR), may allow protection of the conductor and conductor insulation. The above materials may be incorporated within a matrix of polymer (e.g., silicone rubber) within the pace/sense electrode. The outer layer of polymer may even be allowed to abrade as a sacrificial layer before the more abrasion-resistant material stops or significantly impedes further material loss. The key to avoiding abrasion is to limit the contact force and relative motion between the materials. A layer of material may be applied to the pace/sense electrode and/or harness that is expected to abrade and allow the mating materials to “sink into” one another. Thus the contact area between the materials will be increased from an initial point contact between curved surfaces to a more widespread contact surface. The benefit is that the local contact force between the materials will drop, and frictional (abrasive) forces will be reduced. The relative motion between the materials may also be reduced, further reducing potential for abrasion. A further aspect includes use of soft materials on the pace/sense electrodes and cardiac harness. The soft materials “sink into” one another, decreasing contact force and relative movement that can cause abrasion. Similar to constructions mentioned previously, material examples include a low durometer polymer, porous polymer, or brush/carpet-like material. As mentioned previously, any feature that helps secure the harness and pace/sense electrode together and prevent relative motion will help avoid abrasion.

Delivery and implantation of an ICD, CRT-D, pacemaker, leads, and any other device associated with the cardiac rhythm management devices can be performed by means well known in the art. Preferably, the ICD/CRT-D/pacemaker, are delivered through the same minimally invasive access site as the cardiac harness, electrodes, and leads. The leads are then connected to the ICD/CRT-D/pacemaker in a known manner. In one embodiment of the invention, the ICD or CRT-D or pacemaker (or combination device) is implanted in a known manner in the abdominal area and then the leads are connected. Since the leads extend from the apical ends of the electrodes (on the cardiac harness) the leads are well positioned to attach to the power source in the abdominal area.

It may be desired to reduce the likelihood of the development of fibrotic tissue over the cardiac harness so that the elastic properties of the harness are not compromised. Also, as fibrotic tissue forms over the cardiac harness and electrodes over time, it may become necessary to increase the power of the pacing stimuli. As fibrotic tissue increases, the right and left ventricular thresholds may increase, commonly referred to as “exit block.” When exit block is detected, the pacing therapy may have to be adjusted. Certain drugs such as steroids, have been found to inhibit cell growth leading to scar tissue or fibrotic tissue growth. Examples of therapeutic drugs or pharmacologic compounds that may be loaded onto the cardiac harness or into a polymeric coating on the harness, on a polymeric sleeve, on individual undulating strands on the harness, or infused through the lumens in the electrodes and delivered to the epicardial surface of the heart include steroids, taxol, aspirin, prostaglandins, and the like. Various therapeutic agents such as antithrombogenic or antiproliferative drugs are used to further control scar tissue formation. Examples of therapeutic agents or drugs that are suitable for use in accordance with the present invention include 17-beta estradiol, sirolimus, everolimus, actinomycin D (ActD), taxol, paclitaxel, or derivatives and analogs thereof. Examples of agents include other antiproliferative substances as well as antineoplastic, antiinflammatory, antiplatelet, anticoagulant, antifibrin, antithrombin, antimitotic, antibiotic, and antioxidant substances. Examples of antineoplastics include taxol (paclitaxel and docetaxel). Further examples of therapeutic drugs or agents include antiplatelets, anticoagulants, antifibrins, antiinflammatories, antithrombins, and antiproliferatives. Examples of antiplatelets, anticoagulants, antifibrins, and antithrombins include, but are not limited to, sodium heparin, low molecular weight heparin, hirudin, argatroban, forskolin, vapiprost, prostacyclin and prostacyclin analogs, dextran, D-phe-pro-arg-chloromethylketone (synthetic antithrombin), dipyridamole, glycoprotein IIb/IIIa platelet membrane receptor antagonist, recombinant hirudin, thrombin inhibitor (available from Biogen located in Cambridge, Mass.), and 7E-3B® (an antiplatelet drug from Centocor located in Malvern, Pa.). Examples of antimitotic agents include methotrexate, azathioprine, vincristine, vinblastine, fluorouracil, adriamycin, and mutamycin. Examples of cytostatic or antiproliferative agents include angiopeptin (a somatostatin analog from Ibsen located in the United Kingdom), angiotensin converting enzyme inhibitors such as Captopril® (available from Squibb located in New York, N.Y.), Cilazapril® (available from Hoffman-LaRoche located in Basel, Switzerland), or Lisinopril® (available from Merck located in Whitehouse Station, N.J.); calcium channel blockers (such as Nifedipine), colchicine, fibroblast growth factor (FGF) antagonists, fish oil (omega 3-fatty acid), histamine antagonists, Lovastatin® (an inhibitor of HMG-CoA reductase, a cholesterol lowering drug from Merck), methotrexate, monoclonal antibodies (such as PDGF receptors), nitroprusside, phosphodiesterase inhibitors, prostaglandin inhibitor (available from GlaxoSmithKline located in United Kingdom), Seramin (a PDGF antagonist), serotonin blockers, steroids, thioprotease inhibitors, triazolopyrimidine (a PDGF antagonist), and nitric oxide. Other therapeutic drugs or agents which may be appropriate include alpha-interferon, genetically engineered epithelial cells, and dexamethasone.

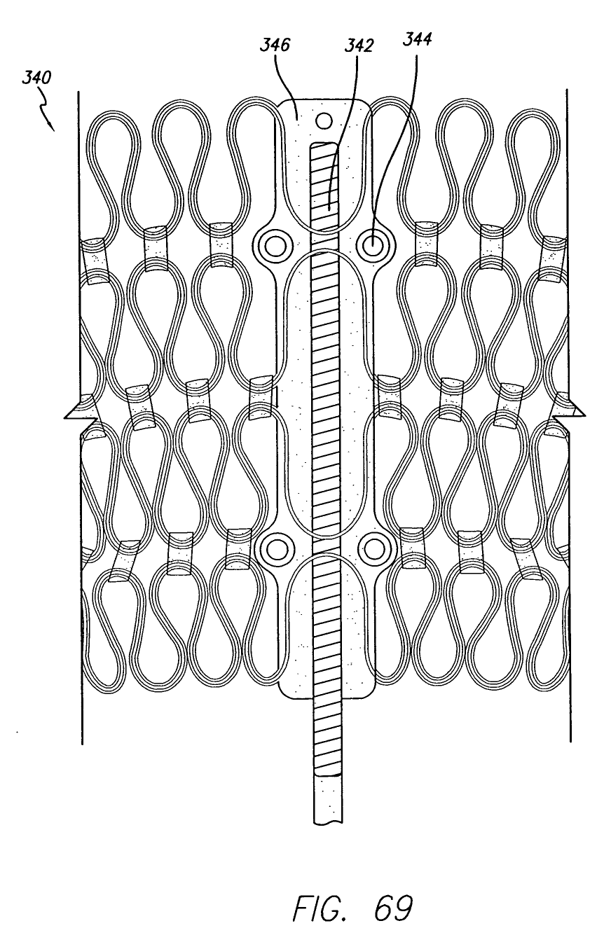

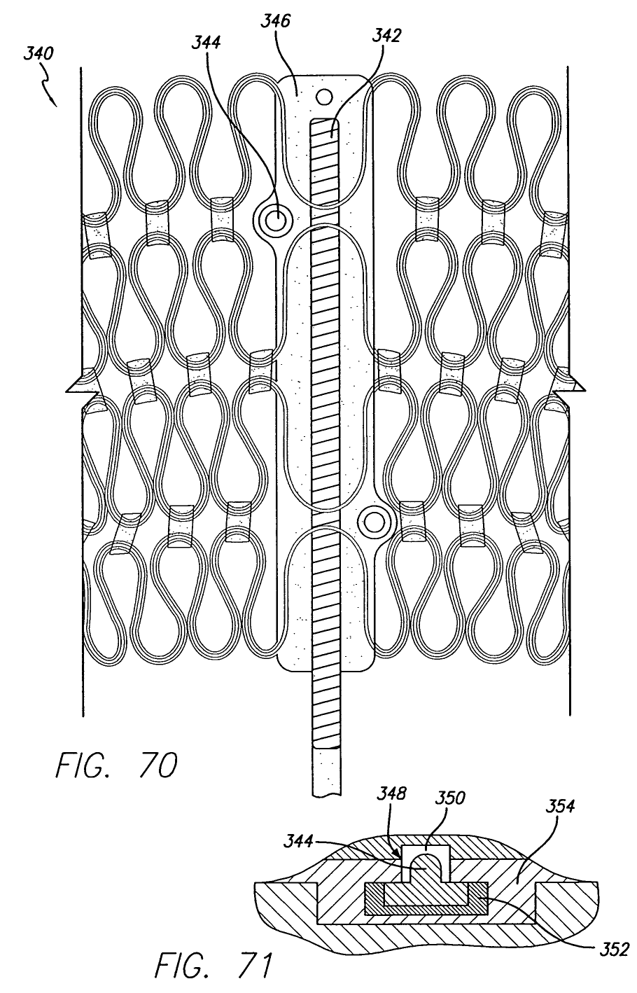

As previously discussed, electrodes may also be positioned on the cardiac harness such as in FIGS. 25A through 26B. FIG. 69 shows another embodiment of a cardiac harness 340 having a defibrillation electrode 342 and pacing/sensing electrodes 344 integrated into a spine 346 of the cardiac harness. This embodiment includes two pairs of pacing/sensing electrodes being retained by the spine. Any number of pacing/sensing electrodes can be retained by the spine, including one electrode, one pair of electrodes as shown in FIG. 70, or three pairs of electrodes. A greater the number of electrode pairs on the cardiac harness provides more positions on the heart where pacing/sensing features may be optimal.

When molding the pacing/sensing electrodes 344 into the spine, a rubber cup 348 having a top cup portion 350 and a bottom cup portion 352 is used. As shown in FIG. 71, the pacing/sensing electrode 344 is captured between the top cup portion and the bottom cup portion and placed into a silicone rubber mold 354. During the molding process, the halves of the rubber cup are pressed together by the mold sealing the porous tip of the electrodes. The rubber cup allows the silicone rubber mold to freely flow around the electrode tip. After the molding is done, the molding surrounding the electrode tip is cut away and the top cup portion of the rubber cup is removed revealing the pacing/sensing electrode. The bottom cup portion may be integrated into the silicone rubber mold. Since the pacing/sensing electrode is not bonded, it is possible for the electrode to emit a steroid, such as dexamethasone sodium phosphate, through a silicone matrix as known in the art.

In February of 2006, a study involving a canine was conducted to evaluate certain embodiments of the cardiac harness being used in connection with pacing/sensing electrodes. There were four basic cardiac harness and pace/sense electrode spine configurations tested during the study. Deployment #1 consisted of a cardiac harness and four modular pacing/sensing spines 306 as shown in FIG. 66, each having one bipolar electrode pair. The spines 306 were co-laced to the delivery system and delivered simultaneously with the cardiac harness. Spine “MA(1)” was positioned basal on the anterolateral wall of the right ventricle, and spine “MA(2)” was located mid-wall on the left posterolateral. Spine “MB” was positioned on the posterolateral wall of the right ventricle. The third spine “MC(1)” was positioned basal on the posterolateral wall of the left ventricle, spine “MC(2)” was located mid-wall on the left posterolateral. Spine “MD” was positioned on the anterolateral wall of the left ventricle. The pacing and sensing performances of the electrode bipole on each spine was evaluated utilizing the diagnostic capabilities of a CRT-D pulse generator. A summary of these results is tabulated in the below table.

Deployment #1 - Performance Results

| | | Sense | Pace | Pace | |
|---|---|---|---|---|---|
| | | Amplitude | Impedance | Threshold | Signal |
| Evaluation # | Spine ID | [mV] | [Ω] | [V] | Quality |
| 1 | MA | >25.0 | 1355 | 4.0 | Good |
| 2 | MC | 20.4 | 1118 | 2.0 | Good |
| 3 | MB | 24.9 | 1229 | 2.4 | Good |
| 4 | MD | 16.1 | 1118 | 2.8 | Good |
| 5 | MA(2) | >25.0 | 1149 | >7.0 | Good |
| 6 | MC(2) | 9.9 | 1168 | 1.8 | Good |

In general, the pace/sense performances of the above four bipoles tested were excellent. All sense amplitudes and pace impedances were well above minimum acceptable levels, and the signal quality of the sensed electrograms were noise-free and robust. Acute pace capture thresholds were slightly higher than desirable (i.e. >2V), however, optimal performance from these prototype pacing/sensing electrodes and associated construction (e.g. stainless steel electrodes with mini-clip adapters) was not expected.

Deployment #2 consisted of a cardiac harness and four modular pacing/sensing spines 280 as shown in FIG. 64, each having one bipolar electrode pair. The spines 280 were independently deployed between the epicardium and the cardiac harness already positioned on the beating canine heart by lacing them to a push-arm. Spine designated “PA” was positioned basal on the left posterolateral, and spine “PB” was positioned basal on the right posterolateral. The third spine “PD” was located basal on the left posterolateral. Spine “PE(1)” was located mid-wall on the right ventricular free wall, and spine “PE(2)” was located basal on the right ventricular free wall. The pacing and sensing performances of the electrode bipole on each spine was evaluated utilizing the diagnostic capabilities of a CRT-D pulse generator. A summary of these results is tabulated in the below table.

Deployment #2 - Performance Results

| | | Sense | Pace | Pace | |
|---|---|---|---|---|---|
| | | Amplitude | Impedance | Threshold | Signal |
| Evaluation # | Spine ID | [mV] | [Ω] | [V] | Quality |
| 7 | PD | 22.6 | 1349 | 2.8 | Inter- |
| | | | | | mittent |
| | | | | | Good |
| 8 | PE(1) | >25.0 | 1355 | >7.5 | Good |
| 9 | PE(2) | 24.1 | 1321 | 2.4 | Good |
| 10 | PA | 7.1 | 988 | 1.2 | Good |
| 11 | PB | 16.6 | 975 | 2.6 | Good |

In general, the pace/sense performances of the above four bipoles tested were very good. All sense amplitudes and pace impedances were well above minimum acceptable levels, and the signal quality of the sensed electrograms were noise-free and robust. However, evaluation #7 encountered variable and intermittent signal amplitude and quality, and the reason for this was not positively determined. Acute pace capture thresholds were slightly higher than desirable (i.e., >2V), and one location (evaluation #8) could not be captured. The probable explanations for these pace capture results mirror those stated above in deployment #1.

Deployment #3 consisted of a cardiac harness with integrated defibrillation and pacing/sensing electrodes, such as the cardiac harness 340 shown in FIG. 69. However, the cardiac harness used in this study had three bipolar pairs of electrodes per spine, with each electrode being designated a number from 1 to 6 starting from the bottom or basal end of the cardiac harness. In the below table, “RA” stands from right anterolateral, “RP” stands from right posterolateral, “LA” stands for left anterolateral, and “LP” stands for left posterolateral. The pacing and sensing performances of the electrode bipoles along each cardiac harness spine was evaluated utilizing the diagnostic capabilities of a CRT-D pulse generator. A summary of these results is tabulated in the below table.

Deployment #3 - Performance Results

| | | | Sense | Pace | | |
|---|---|---|---|---|---|---|
| | | Bipole | Ampli- | Imped- | Pace | |
| Evalu- | Bipole | Epicardial | tude | ance | Threshold | Signal |
| ation# | ID | Location | [mV] | [Ω] | [V] | Quality |
| 12 | RA 1, 2 | RA basal | 12.9 | 905 | 1.8 | Variable |
| 13 | LA 1, 2 | LP basal | 3.0 | 797 | >7.0 | Variable |
| 14 | RA 3, 4 | RA mid- | 15.0 | 1034 | 1.4 | Good |
| | | wall | | | | |
| 15 | LA 3, 4 | LP mid- | 18.9 | 1168 | 1.8 | Good |
| | | wall | | | | |
| 16 | RA 5, 6 | RA apical | >25 | 1055 | 1.4 | Good |
| 17 | LA 5, 6 | LP apical | 16.0 | >2000 | >7.5 | Noisy |
| 18 | RB 1, 2 | RP basal | 6.5 | 939 | 3.0-3.5 | Good |
| 19 | LB 1, 2 | LA basal | 3.8 | 645 | 4.0 | Good |
| 20 | RB 3, 4 | RP mid- | >25.0 | 1258 | 2.4 | Good |
| | | wall | | | | |
| 21 | LB 3, 4 | LA mid- | 12.0 | 1072 | 1.2 | Good |
| | | wall | | | | |
| 22 | RB 5, 6 | RP apical | 13.3 | 1643 | >7.5 | Noisy/ |
| | | | | | | variable |
| 23 | LB 5, 6 | LA apical | 11.0 | 1028 | 1.2-1.4 | Good |

All unacceptable and marginal values were derived from basal and apical bipole locations only. In contrast, all four of the mid-wall bipole locations provided excellent pace/sense performance with consistently robust signal quality. The reasons for the relatively poorer performance of some of the basal and apical bipoles were not determined, but one hypothesis is that the affected bipoles were not making good contact with the myocardium because of the intervening fat/vessel.

Deployment #4 was similar to deployment #3 except that the cardiac harness included 20% barium-loaded posterior row connectors. Further after the cardiac harness was positioned on the heart, a modular pacing/sensing spine 280, such as the one shown in FIG. 64, was deployed between the cardiac harness, epicardium, and the two left ventricular harness electrode spines, and was advanced until it was positively positioned on the epicardium of the left atrium. The modular spine is designated “PC” in the table below. Further, “RA” stands from right anterolateral, “RP” stands from right posterolateral, “LA” stands for left anterolateral, and “LP” stands for left posterolateral. The pacing and sensing performances of the electrode bipoles along each cardiac harness spine was evaluated utilizing the diagnostic capabilities of a CRT-D pulse generator. A summary of these results is tabulated in the below table.

Deployment #4 - Performance Results

| | | | | | Pace | |
|---|---|---|---|---|---|---|
| | | Bipole | Sense | Pace | Thresh- | |
| Evalu- | Bipole | Epicardial | Amplitude | Impedance | old | Signal |
| ation# | ID | Location | [mV] | [Ω] | [V] | Quality |
| 24 | RA 3, 4 | RA mid- | 8.6 | 1174 | 1.8 | Good |
| | | wall | | | | |
| 25 | LA 3, 4 | LP mid- | 16.1 | 1072 | 1.4 | Good |
| | | wall | | | | |
| 26 | RB 3, 4 | RP mid- | 9.8 | 1201 | 1.0 | Good |
| | | wall | | | | |
| 27 | LB 3, 4 | LA mid- | 20.6 | 1168 | 2.4 | Good |
| | | wall | | | | |
| 28 | PC | Left | 7.6 | 693 | 2.8 | Good |
| | | atrium | | | | |
| 29 | RB 3, 4 | RP mid- | 12.3 | 994 | Not | Not ap- |
| | | wall | | | appli- | plicable |
| | | | | | cable | |
| 30 | LB 3, 4 | LA mid- | 10.6 | 1072 | Not | Not ap- |
| | | wall | | | appli- | plicable |
| | | | | | cable | |

In general, the pacing/sensing performances of the bipoles tested were very good. Sense amplitudes and pace impedances were well above minimum acceptable levels, and the signal quality of the sensed electrograms were generally noise-free and robust. All pace capture thresholds were also acceptable.

Although the present invention has been described in terms of certain preferred embodiments, other embodiments that are apparent to those of ordinary skill in the art are also within the scope of the invention. Accordingly, the scope of the invention is intended to be defined only by reference to the appended claims. While the dimensions, types of materials and coatings described herein are intended to define the parameters of the invention, they are by no means limiting and are exemplary embodiments.

What is claimed:

1. A moveable electrode spine, comprising:
a spine body having a first surface and a second surface, and the spine body having a low profile;
at least one pace/sense electrode retained by the spine body; and
the spine body being formed from a dielectric material.

2. The spine of claim 1, wherein the second surface of the body includes a layer of ePTFE.

3. The spine of claim 1, wherein the at least one pace/sense electrode is one bipolar electrode pair.

4. The spine of claim 3, wherein the bipolar electrode pair is longitudinally positioned within the body.

5. The spine of claim 3, wherein the bipolar electrode pair is horizontally positioned within the body.

6. The spine of claim 1, wherein spine body is paddle shaped.

7. The spine of claim 1, wherein spine body is circular shaped.

8. The spine of claim 1, wherein at least one pace/sense electrode is an Omni directional bipolar electrode pair.

9. The spine of claim 1, wherein the spine body being formed of silicone rubber.

10. The spine of claim 1, further comprising grip pads disposed on the first surface of the spine body.

11. A system for treating the heart, comprising:
a cardiac harness configured to conform generally to at least a portion of a heart; and
a first moveable structure having a body for retaining an electrode configured for placement between the cardiac harness and the surface of the heart.

12. The system of claim 11, wherein the first moveable structure retains a pair of bipolar electrodes for providing pacing/sensing functions to the heart.

13. The system of claim 11, further comprising a second moveable structure having a body for retaining an electrode configured for placement between the cardiac harness and the surface of the heart.

14. The system of claim 13, wherein the first moveable structure and the second moveable structure each retain one electrode for providing pacing/sensing functions to the heart.

15. The system of claim 13, wherein the first moveable structure and the second moveable structure each retain two electrodes for providing pacing/sensing functions to the heart.

16. The system of claim 11, wherein the body of the first moveable structure retains a defibrillation electrode for providing a defibrillating shock through the heart.

17. A method for pacing/sensing a beating heart, comprising:
inserting the cardiac harness through a minimally invasive access site and around at least a portion of the heart; and
inserting a moveable structure having a body retaining an electrode through the minimally invasive access site and positioning the moveable structure between the cardiac harness and the epicardium of the heart, wherein compressive forces of the cardiac harness hold the moveable structure in position on the heart.

18. The method of claim 17, wherein inserting the cardiac harness and inserting moveable structure occurs simultaneously on a delivery device for carrying the cardiac harness and the moveable structure.

19. The method of claim 17, wherein inserting the cardiac harness on a delivery device for carrying the cardiac harness, and inserting the moveable structure on a push arm after the cardiac harness is positioned on the heart.

20. The method of claim 17, further comprising scouting a position for the moveable structure on the surface of the heart.

21. The method of claim 17, further comprising making an incision in the pericardium so that the cardiac harness and moveable structure are mounted on the epicardial surface of the heart and under the pericardium.

22. The method of claim 17, wherein inserting the moveable structure, the body retains a pair of bipolar electrodes for providing pacing/sensing functions to the heart.

23. The method of claim 17, wherein inserting the moveable structure, the body retains a defibrillation electrode for providing a defibrillating shock through the heart.

24. The method of claim 17, further comprising inserting multiple moveable structures each having a body retaining an electrode through the minimally invasive access site and positioning each moveable structure between the cardiac harness and the epicardium of the heart, wherein compressive forces of the cardiac harness hold the moveable structures in place on the heart.

25. The method of claim 17, further comprising attaching the electrode to the body of the moveable structure in a sterile field before inserting the moveable structure through the minimally invasive access site.
